(19) United States
(12) Patent Application Publication
(10) Pub. No.: US 2025/0304715 A1
(43) Pub. Date: Oct. 2, 2025

(54) COMPOSITIONS AND METHODS FOR UPREGULATING HLA CLASS I ON TUMOR CELLS

(71) Applicant: University of Utah Research Foundation, Salt Lake City, UT (US)

(72) Inventors: Fiorella Iglesias, Salt Lake City, UT (US); Djordje Atanackovic, Salt Lake City, UT (US); Tim Leutkens, Salt Lake City, UT (US)

(21) Appl. No.: 19/237,233

(22) Filed: Jun. 13, 2025

Related U.S. Application Data
(63) Continuation of application No. 17/778,317, filed on May 19, 2022.
(60) Provisional application No. 62/940,689, filed on Nov. 26, 2019.

(51) Int. Cl. C07K 16/30; A61K 39/00; A61K 40/11; A61K 40/32; A61K 40/42; A61P 35/00; C07K 14/47; C07K 16/28

(52) U.S. Cl. CPC C07K 16/3084; A61K 40/11; A61K 40/32; A61K 40/4211; A61K 40/4258; A61K 40/4269; A61K 40/427; A61P 35/00; C07K 14/4705; C07K 16/2803; A61K 2039/505; A61K 2239/31; A61K 2239/38; A61K 2239/47; C07K 2317/622; C07K 2319/03; C07K 2319/41; C07K 2319/42; C07K 2319/43

(57) ABSTRACT

Disclosed are nucleic acid constructs comprising a promoter; a nucleic acid sequence encoding a single-chain variable fragment (scFv); a nucleic acid sequence encoding a notch transmembrane domain; and a nucleic acid sequence encoding a transcription factor. Disclosed are vectors comprising any of the disclosed nucleic acid constructs. Disclosed are proteins comprising a scFv; a notch transmembrane domain; and a transcription activator. Disclosed are methods of increasing human leukocyte antigen class I (HLA-I) on the surface of a tumor cell in a subject comprising administering to the subject one or more of the recombinant cells or compositions comprising a recombinant cell disclosed herein.

### CROSS-REFERENCE TO RELATED APPLICATIONS

This application is a Continuation of U.S. patent application Ser. No. 17/778,317, filed May 19, 2022, which is a 371 of International Application PCT/US2020/062371, filed Nov. 25, 2020, which claims the benefit of U.S. Provisional Patent Application No. 62/940,689, filed on Nov. 26, 2019, incorporated by reference herein in its entirety.

### REFERENCE TO SEQUENCE LISTING

The Sequence Listing submitted Jun. 13, 2025 as a text file named “21101.0388U3.xml,” created on Jun. 13, 2025, and having a size of 121,483 bytes is hereby incorporated by reference pursuant to 37 C.F.R. § 1.52(e)(5).

### BACKGROUND

HLA class I loss is a common immune escape mechanism present in many tumors, including some of the most common tumor types, such as colorectal and lung cancer. This is significant because the loss of HLA not only renders the tumor cells invisible to the patient's own immune system but also to adoptive T cell therapies using T cells transduced with tumor-specific T cell receptors. Importantly, the majority of tumors start out HLA class I positive but, over time, cells with low or negative HLA expression are selected by the patients' tumor-reactive T cells. This indicates that highly effective anti-tumor T cells are in fact present in those patients with the most dramatic HLA loss. Restoring HLA expression in these patients has the potential to dramatically enhance spontaneous and adoptive anti-tumor immunity. This principle is similar to immune checkpoint inhibition, which enables the patients' own T cells to react to tumor cells. While different approaches have been shown to induce HLA expression in tumor cells, such as systemic delivery of interferon gamma (IFNG or IFNγ), none of these strategies are effective and safe. Currently, no therapies are approved for the induction of HLA class I.

### BRIEF SUMMARY

Described herein are new approaches to induce HLA expression in tumor cells using a targeted cellular therapy, combining state-of-the-art T cell engineering principles with tumor-specific antibodies, that could be used either as a monotherapy or in combination with any adoptive TCR-transgenic T cell approach. Specifically, disclosed herein are methods, compositions and systems that can utilize the transmembrane region of Notch, which is cleaved physiologically when the extracellular Notch domain binds its ligand and thereby releases the intracellular Notch domain. The intracellular domain then translocates to the cell nucleus and activates a transcriptional program. By replacing the extracellular domain with antibody domains and the intracellular domain with non-human transcription factors, it is possible to drive expression of custom cellular programs in response to specific binding events.

Described herein is the first targeted approach to upregulate HLA specifically on tumor cells through targeted delivery of IFNG by engineered T cells using the synthetic Notch (synNotch) system. Treatment of tumor cells with the disclosed engineered T cells strongly upregulates HLA despite the secretion of extremely low levels of IFNG. Induction of HLA in turn enhances tumor cell killing by tumor-specific T cells.

Surprisingly, the compositions and methods disclosed herein can allow for the expression and secretion of IFNG at levels high enough to upregulate HLA.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1C show an induction of HLA class I surface expression in neuroblastoma cells using recombinant and T cell-derived IFNG. (A) HLA-ABC or HLA-A2 expression on neuroblastoma cell lines before and after treatment with 500 IU/mL of recombinant IFNG for 48 hours was determined by flow cytometry. (B) IFNG ELISA of culture supernatants of J76 cells constitutively expressing GFP or IFNG at day 1, day 2, and day 3 after completely replacing culture medium. (C) HLA class I expression on neuroblastoma cell line Kelly after 24 h of co-culture with J76 cell constitutively expressing GFP or IFNG.

FIGS. 2-2E show development of neuroblastoma-specific synNotch-IFNG cells. (A) Schematic of IFNG-synNotch approach. SynNotch receptors binding to a neuroblastoma antigen, release a transcription factor that induces the expression of IFNG, which in turn upregulates HLA class I on neuroblastoma cells, sensitizing them for killing by tumor-specific T cells. (B) Constructs used for the conditional expression of IFNG by synNotch T cells and conditional expression of BFP. Construct I: synNotch receptor containing the constitutive PGK promoter, followed by a neuroblastoma-specific single-chain variable fragment (scFv), a myc tag allowing detection of the receptor's surface expression by flow cytometry, the core Notch1 domain, which upon antigen binding releases the intracellular chimeric transcription factor Gal4-VP64. Construct II: Response construct containing a minimal CMV promoter controlled by Gal4 upstream activation sequences (UAS) that can be specifically induced by the Gal4-VP64 transcription factor. This inducible promoter controls expression of the IFNG open-reading frame, as well as an internal ribosome entry site (IRES) that facilitates expression of the fluorescent reporter mCherry, which in turn allows detection of cells showing synNotch signaling by flow cytometry. (C) Expression of IFNG by synNotch cells recognizing CD19 when co-cultured when CD19+ Daudi cells or CD19− Kelly cells at different effector-target ratios as determined by IFNG ELISA. (D) SynNotch receptor surface expression (myc) and baseline reporter expression (mCherry) in J76 cells expressing different neuroblastoma-specific synNotch receptors. (E) Expression of CD19 and GD2 in lymphoma cell lines (red) or neuroblastoma cell lines compared to fluorescence-minus-one controls.

FIGS. 3A-3H show conditional expression of IFNG by genetically engineered T cells and induction of HLA class I on neuroblastoma cells. (A) IFNG levels in the supernatants of co-cultures of IFNG-synNotch cells recognizing CD19 or GD2 with neuroblastoma cell lines NB1643 and Kelly after 8 hours, 24 hours and 48 hours, as determined by ELISA. (B) IFNG ELISpot of co-cultures of the GD2+ neuroblastoma cell line Kelly or CD19+ B cell lymphoma cell line Daudi with synNotch cells and IFNG-synNotch cells recognizing CD19 or GD2. (C) Cytokine concentrations in 48 hours co-cultures of IFNG-synNotch cells recognizing CD19 or GD2 with the GD2 positive cell line NB1643 and Kelly, and the GD2 negative cell line Daudi, as determined by cytometric bead array. (D) Expression of HLA class I on neuroblastoma cell line Kelly treated with conditioned supernatants from indicated 48 hours co-cultures. (E) Expression of HLA class I on neuroblastoma cell lines Kelly and (F) NB1643 after 48 h co-culture with CD19- or GD2-specific IFNG-synNotch cells as determined by flow cytometry. (G) Expression of PD-L1 on Kelly cells after 48 h co-culture with GD2 synNotch cells as determined by flow cytometry. (H) Expression of PD-1 on GD2 synNotch cells after 48 h co-culture with Kelly cells as determined by flow cytometry.

FIGS. 4A-4C shows killing of neuroblastoma cell line SK-N-DZ by tumor antigen-specific T cells after pretreatment with snGD2 T cells. (A) Schematic of the PRAME-specific T cell receptor construct. (B) Expression of HLA-ABC in the HLA-A2+ and PRAME+ neuroblastoma cell line SK-N-DZ, transduced with an NY-ESO-1 expression construct, before and after treatment with snCD19 or snGD2 cells. (C) Killing of SK-N-DZ sells transduced with firefly luciferase cells after pretreatment with snCD19 or snGD2 T cells followed by treatment with PRAME-specific HSS1 T cells, NY-ESO-1-specific 1G4 T cells, or control T cells expressing GFP. Cytotoxicity was determined by luminescence assay.

FIGS. 5A and 5B show HLA induction in vivo using synNotch T cells (A) A total of 1×10⁶ Kelly cells were injected subcutaneously with Matrigel into immunocompromised NSG mice. Once tumors reached a diameter of 5 mm, 1×10⁶ snCD19 or snGD2 T cells were injected in PBS into the tumor. Immunohistochemistry for HLA-ABC was performed on paraffin-embedded tumor sections. (B) Total IFNG levels in the peripheral blood of the same animals were determined by ELISA.

FIG. 6 shows NY-ESO 1 and PRAME mRNA expression in neuroblastoma cell lines. Expression of CGA and housekeeping gene GAPDH was determined in positive control myeloma cell line U266 as well as testis and neuroblastoma cell lines by RT-PCR. HLA-A2 status was determined by PCR.

FIGS. 7A-7C show transgene expression, activation, and cytotoxic activity of NY-ESO-1-specific TCR-transduced T cells. Human T cells with (left) or without (right) an endogenous TCR were sequentially transduced with NY-ESO-1-specific TCR alpha and beta chains. (A) Reporter gene expression as well as tetramer binding was determined by flow cytometry. (B) IFNγ secretion by NY-ESO-1-TCR transduced T cells after incubation of antigen-presenting cells pulsed with the corresponding NY-ESO-1 peptide as determined by ELISpot. (C) Cytotoxicity of human T cells transduced with both alpha/beta (ab) or only the alpha chain (a) of an NY-ESO-1-specific TCR against HLA-A2/NY-ESO-1-positive melanoma cell line A375 and NY-ESO-1-negative cell line K562 as determined by flow cytometry.

FIG. 8 shows a schematic of HLA upregulation strategies in neuroblastoma cells.

FIGS. 9A and 9B show high-throughput luciferase cytotoxicity assay. (A) Schematic of cytotoxicity assay. (B) Results of cytotoxicity assay using specific TCR T or control T cells as determined by luminescence.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a nucleic acid construct” includes a plurality of such nucleic acid constructs, reference to “the nucleic acid sequence” is a reference to one or more nucleic acid sequences and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

As used herein, the term “wild-type” refers to a gene or gene product which has the characteristics of that gene or gene product when isolated from a naturally-occurring source.

The term “percent homology” or “% homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res.25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nucleic Acid Constructs

### 1. Synthetic Notch (SynNotch) Constructs

Disclosed are nucleic acid constructs comprising a promoter; a nucleic acid sequence encoding a single-chain variable fragment (scFv); a nucleic acid sequence encoding a notch transmembrane domain; and a nucleic acid sequence encoding a transcription factor.

In some aspects, any of the disclosed promoters, nucleic acid sequences encoding a scFv, nucleic acid sequences encoding a notch transmembrane domain, and nucleic acid sequences encoding a transcription factor can be present in the disclosed nucleic acid constructs.

i. Promoter

The disclosed nucleic acid constructs can comprise a promoter. Examples of promoters that can be present in the nucleic acid constructs disclosed herein are given throughout the specification. Examples of promoters present in the disclosed nucleic acid constructs can include, but are not limited to, CMV based, CAG, SV40 based, heat shock protein, a mH1, a hH1, chicken β-actin, U6, Ubiquitin C, or EF-1α promoters.

Promoters for controlling transcription from vectors in mammalian host cells can be obtained from various sources, for example, the genomes of viruses such as polyoma, Simian Virus 40 (SV40), adenovirus, retroviruses, hepatitis B virus and most preferably cytomegalovirus, or from heterologous mammalian promoters, e.g., β-actin promoter. The early and late promoters of the SV40 virus are conveniently obtained as an SV40 restriction fragment, which also contains the SV40 viral origin of replication (Fiers et al., Nature, 273:113 (1978) which is incorporated by reference herein in its entirety for viral promoters). The immediate early promoter of the human cytomegalovirus is conveniently obtained as a HindIII E restriction fragment (Greenway, P. J. et al., Gene 18:355 360 (1982) which is incorporated by reference herein in its entirety for viral promoters). Of course, promoters from the host cell or related species also are useful herein, and can be used for tissue specific gene expression or tissues specific regulated gene expression. The cited references are incorporated herein by reference in their entirety for their teachings of promoters.

The disclosed nucleic acid constructs disclosed herein can further comprise an enhancer. “Enhancer” generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Each of the cited references is incorporated herein by reference in their entirety for their teachings of enhancers. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). Each of the cited references is incorporated herein by reference in their entirety for their teachings of potential locations of enhancers. They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100 270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In some aspects, the promoter and/or enhancer region can act as a constitutive promoter and/or enhancer to maximize expression of the region of the transcription unit to be transcribed. In certain constructs the promoter and/or enhancer region are active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

Disclosed are nucleic acid constructs comprising a promoter; a nucleic acid sequence encoding a scFv; a nucleic acid sequence encoding a notch transmembrane domain; and a nucleic acid sequence encoding a transcription factor, wherein the promoter is a constitutive promoter. Constitutive promoters are well-known in the art. Examples of constitutive promoters include, but are not limited to, a PGK promoter, CMV promoter, SV40 promoter, EF1A promoter, SFFV promoter, Ubc promoter, and CAG promoter.

As described herein, in some aspects, the promoter can be a regulatable promoter. Regulatable promoters are well-known in the art. Examples of regulatable promoters include, but are not limited to, tetracycline-regulated, arabinose-inducible promoter, and lactose promoter system.

ii. Single-Chain Variable Fragment

The nucleic acid constructs described herein can comprise a nucleic acid sequence encoding a single-chain variable fragment (scFv). “Single-chain variable fragment”, “Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Pluckthun in The Pharmacology of Monoclonal Antibodies, Rosenburg and Moore eds. Springer-Verlag, N.Y. (1994), 269-315.

In some aspects, a nucleic acid sequence encoding a scFv can be used to target a gene product resulting from the disclosed nucleic acid constructs to a target/cell of interest.

Disclosed are nucleic acid constructs comprising a promoter; a nucleic acid sequence encoding a scFv; a nucleic acid sequence encoding a notch transmembrane domain; and a nucleic acid sequence encoding a transcription factor, wherein the scFv is a tumor specific scFv.

In some aspects, the scFv is a neuroblastoma-specific scFv. In some aspects, the scFv is a GD2 specific ScFv.

In some aspects, the scFv is a scFv specific for CD3, CD5, CD7, CD19, CD30, CD33, CD38, CD123, CD133, CD229, BCMA, c-Met, CEA, EGFR, EGFRVIII, EpCAM, GD2, HER1, HER2, LINGO1, mesothelin, or MUC1.

In some aspects, the scFv comprises a heavy chain fragment and a light chain fragment. In some aspects, the scFv can comprise a heavy chain fragment comprising an amino acid sequence of

QVKLQQSGPELVEPGASVKISCKTSGYKFTEYTMHWVKQSHGKSL
EWIGGINPNNGGTNYKQKFKGKATLTVDKSSSTAYMELRSLTSED
SAVYYCARDTTVPFAYWVQGTTVTVSS;

and a light chain fragment comprising an amino acid sequence of

DIELTQSPAIMSASPGEKVTMTCSGSSSISYMHWYQQKPVTSPKR
WIYDTSKLASGVPARFSGSGSGTSYSLTISSMEAVDAATYYCHQR
SSYPLTFGAGTQLEIKR.

In some aspects, the scFv can comprise a heavy chain fragment comprising an amino acid sequence of

EVQLVQSGAEVEKPGASVKISCKASGSSFTGYNMNWVRQNIGKSL
EWIGAIDPYYGGTSYNQKFKGRATLTVDKSTSTAYMHLKSLRSED
TAVYYCVSGMEYWGQGTSVTVSS;

and a light chain fragment comprising an amino acid sequence of

DVVMTQTPLSLPVTPGEPASISCRSSQSLVHRNGNTYLHWYLQKP
GQSPKLLIHKVSNRFSGVPDRFSGSGSGTDFTLKISR VEAEDLG
VYFCSQSTHVPPLTFGAGTKLELKR.

In some aspects, the scFv can comprise a heavy chain fragment comprising an amino acid sequence of

QVQLVESGPGVVQPGRSLRISCAVSGFSVTNYGVHWVRQPPGKGL
EWLGVIWAGGITNYNSAFMSRLTISKDNSKNTVYLQMNSLRAEDT
AMYYCASRGGHYGYALDYWGQGTLVTVSS;

and a light chain fragment comprising an amino acid sequence of

EIVMTQTPATLSVSAGERVTITCKASQSVSNDVTWYQQKPGQAPR
LLIYSASNRYSGVPARFSGSGYGTEFTFTISSVQSEDFAVYFCQQ
DYSSFGQGTKLEIKR.

In some aspects, the scFv can comprise a heavy chain fragment comprising an amino acid sequence of

QVQLQESGPGLVKPSQTLSITCTVSGFSLASYNIHWVRQPPGKGL
EWLGVIWAGGSTNYNSALMSRLTISKDNSKNQVFLKMSSLTAADT
AVYYCAKRSDDYSWFAYWGQGTLVTVSS;

and a light chain fragment comprises an amino acid sequence of

ENQMTQSPSSLSASVGDRVTMTCRASSSVSSSYLHWYQQKSGKAP
KVWIYSTSNLASGVPSRFSGSGSGTDYTLTISSLQPEDFATYYCQ
QYSGYPITFGQGTKVEIKR.

In some aspects, the scFv can comprise a heavy chain fragment comprising an amino acid sequence of

LISEEDLEVQLVETGGGVVKPGGSLRLSCAASGFTFSDYYMSWIR
QAPGKGLEWVSYISSSGSTIYYADSVKGRFTISRDNSKNTLYLQM
NSLRAEDTAVYYCARESGYDYVFDYWGQGTLVAVSS;

and a light chain fragment comprising an amino acid sequence of

DIQMTQSPSTLSAFVGDRVTITCRASQSISSWLAWYQQKPGKAPK
LLIYAASTLQSGVPSRFSGSGSGTEFTLTISSLQPEDFATYYCQQ
LNSYPITFGQGTRLEIKRILDYSF.

In some aspects, the scFv can comprise a heavy chain fragment comprising an amino acid sequence of

LISEEDLEVQLVESGGGLVQPGGSLRLSCAASGFTFSSFGMHWVR
QAPGKGLEWVAYISSDSSAIYYADTVKGRFTISRDNAKNSLYLQM
NSLRDEDTAVYYCGRGRENIYYGSRLDYWGQGTTVTVSS;

and a light chain fragment comprises an amino acid sequence of

DIQLTQSPSFLSASVGDRVTITCKASQNVDTNVAWYQQKPGKAPK
ALIYSASYRYSGVPSRFSGSGSGTDFTLTISSLQPEDFATYYCQQ
YNNYPFTFGQGTKLEIKILDYSF.

In some aspects, the scFv can comprise a heavy chain fragment comprising an amino acid sequence of

LISEEDLQVQLQQPGAELVKPGASVKLSCKASGYTFTGYWMHWVK
QRPGHGLEWIGEINPSNGRTNYNERFKSKATLTVDKSSTTAFMQL
SGLTSEDSAVYFCARDYYGTSYNFDYWGQGTTLTVSS;

and a light chain fragment comprises an amino acid sequence of

DIQMTQSSSSFSVSLGDRVTITCKANEDINNRLAWYQQTPGNSPR
LLISGATNLVTGVPSRFSGSGSGKDYTLTITSLQAEDFATYYCQQ
YWSTPFTFGSGTELEIKVEILDYSF.

In some aspects, the nucleic acid sequence encoding a single-chain variable fragment can encode a variant of a scFv heavy or light chain sequences provided herein.

iii. Linkers

In some aspects, the disclosed nucleic acid constructs can comprise a nucleic acid sequence that encodes a linker. In some aspects, the disclosed nucleic acid constructs can comprise a nucleic acid sequence that encodes a linker, wherein the sequence is located between nucleic acid sequences that encode a heavy chain fragment and light chain fragment. As a result, the heavy chain fragment and light chain fragment of the disclosed scFvs can be joined via a linker.

In some aspects, the linker comprises an amino acid sequence of GGGGSGGGGSGGGGS. In some aspects, the linker comprises an amino acid sequence of GGGGSGGGGSGGGGSGGGGS. In some aspects, the linker comprises an amino acid sequence of (GGGGS)ₙ, wherein n is 1, 2, 3, 4, 5, 6, 7, 8, 9, or 10. In some aspects, the linker comprises an amino acid sequence of GGSSRSSSSGGGGSGGGG.

In some aspects, a linker can be between 10-40 amino acids in length. In some aspects, a linker can be about 15-20 amino acids in length.

iv. Notch Transmembrane Domain

Disclosed herein are nucleic acid constructs that comprise a nucleic acid sequence encoding a notch transmembrane domain. In some aspects, a nucleic acid sequence encoding a notch transmembrane domain can be used as a transcription activating domain. Upon binding of a scFv, attached upstream of the notch transmembrane domain, to its target, the notch transmembrane domain can be cleaved which allows it to translocate to the nucleus of the cell it is in which ultimately results in delivery of a transcription activator bound to the notch transmembrane domain.

In some aspects, the nucleic acid sequence encoding a notch transmembrane domain encodes a notch transmembrane domain comprising the sequence of

ILDYSFTGGAGRDIPPPQIEEACELPECQVDAGNKVCNLQCNNHA
CGWDGGDCSLNFNDPWKNCTQSLQCWKYFSDGHCDSQCNSAGCLF
DGFDCQLTEGQCNPLYDQYCKDHFSDGHCDQGCNSAECEWDGLDC
AEHVPERLAAGTLVLVVLLPPDQLRNNSFHFLRELSHVLHTNVVF
KRDAQGQQMIFPYYGHEEELRKHPIKRSTVGWATSSLLPGTSGGR
QRRELDPMDIRGSIVYLEIDNRQCVQSSSQCFQSATDVAAFLGAL
ASLGSLNIPYKIEAVKSEPVEPPLPSQLHLMYVAAAAFVLLFFVG
CGVLLSRKRRR

or a variant thereof.

In some aspects, the disclosed nucleic acid constructs further comprise a nucleic acid sequence that encodes one or more EGF repeat (ERR) sequences. The ERR sequences can be from the extracellular domain of Notch. Thus, in some aspects, the Notch transmembrane domain can be extended past just the transmembrane to include the ERR sequences of the extracellular domain. The presence of the ERR sequences can help reduce high basal levels of transcriptional activity when the scFv target is not present. In some aspects, the ERR sequence comprises the amino acid sequence of

PCVGSNPCYNQGTCEPTSENPFYRCLCPAKFNGLLCH.

v. Transcription Factors

The disclosed nucleic acid constructs can comprise a nucleic acid sequence encoding a transcription factor. In some aspects, the transcription factor is a transcription activator. Transcription activators are well known in the art. In some aspects, the transcription activator comprises Gal4. In some aspects, the transcription activator comprises a Gal4-VP64 fusion protein. In some aspects, the Gal4-VP64 fusion protein comprises the amino acid sequence of

MKLLSSIEQACDICRLKKLKCSKEKPKCAKCLKNNWECRYSPKTK
RSPLTRAHLTEVESRLERLEQLFLLIFPREDLDMILKMDSLQDIK
ALLTGLFVQDNVNKDAVTDRLASVETDMPLTLRQHRISATSSSEE
SSNKGQRQLTVSAAAGGSGGSGGSDALDDFDLDMLGSDALDDFDL
DMLGSDALDDFDLDMLGSDALDDFDLDMLGS.

Transcription activators, such as Gal4, can bind to certain nucleic acid regions upstream of a gene (Upstream Activation Sequence (UAS)) and activate transcription of that gene. For example, in some aspects of the current invention, Gal4 binds to a UAS upstream of an interferon gamma (IFNγ) gene resulting in expression of IFNγ.

vi. Detection Agents

In some aspects, the disclosed nucleic acid constructs can comprise a nucleic acid sequence encoding a detection agent. In some aspects, the presence of a detection agent allows for visual detection or purification of the disclosed nucleic acid constructs or the products thereof. For example, a detection agent can be, but is not limited to, a myc tag, his tag, fluorescent tag, FLAG tag, or hemagglutinin tag.

In some aspects, the detection agent can be Mcherry, wherein the amino acid sequence comprises

MVSKGEEDNMAIIKEFMRFKVHMEGSVNGHEFEIEGEGEGRPYEG
TQTAKLKVTKGGPLPFAWDILSPQFMYGSKAYVKHPADIPDYLKL
SFPEGFKWERVMNFEDGGVVTVTQDSSLQDGEFIYKVKLRGTNFP
SDGPVMQKKTMGWEASSERMYPEDGALKGEIKQRLKLKDGGHYDA
EVKTTYKAKKPVQLPGAYNVNIKLDITSHNEDYTIVEQYERAEGR
HSTGGMDELYK.

In some aspects, the detection agent can be tagBFP, wherein the amino acid sequence comprises

MSELIKENMHMKLYMEGTVDNHHFKCTSEGEGKPYEGTQTMRIKV
VEGGPLPFAFDILATSFLYGSKTFINHTQGIPDFFKQSFPEGFTW
ERVTTYEDGGVLTATQDTSLQDGCLIYNVKIRGVNFTSNGPVMQK
KTLGWEAFTETLYPADGGLEGRNDMALKLVGGSHLIANIKTTYRS
KKPAKNLKMPGVYYVDYRLERIKEANNETYVEQHEVAVARYCDLP
SKLGHKLN.

In some aspects, a detection agent can comprise a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

vii. Signaling Peptide

In some aspects, the disclosed nucleic acid constructs can comprise a signaling sequence that encodes a signaling peptide. In some aspects, a signaling peptide can be referred to as a localization signal or sequence or a leader sequence. In some aspects, the signaling peptide can translocate the peptide encoded by the disclosed nucleic acid constructs to the cell membrane.

In some aspects, the signaling peptide is located at the N-terminus of the peptide encoded by the disclosed nucleic acid constructs.

Disclosed are nucleic acid constructs comprising a promoter; a nucleic acid sequence encoding a scFv; a nucleic acid sequence encoding a notch transmembrane domain; a nucleic acid sequence encoding a transcription factor; and a nucleic acid sequence encoding a signaling peptide, wherein each nucleic acid sequence is operably linked to the nucleic acid sequence directly upstream of it. In some aspects, nucleic acid sequence encoding a signaling peptide can be operably linked to a nucleic acid sequence encoding a transcription factor.

Examples of signaling peptides can be, but are not limited to, signaling peptides derived from immunoglobulin heavy chain (e.g. MDWTWRVFCLLAVAPGAHS), immunoglobulin kappa (e.g. MVLQTQVFISLLLWISGAYG) or lambda (e.g. MAWALLLLSLLTQGTGSWA) light chains, CD8 (MALPVTALLLPLALLLHAARP), CD28 (MLRLLLALNLFPSIQVTG), or interleukin-2 (MYRMQLLSCIALSLALVTNS).

viii. Example Constructs

In some aspects, a nucleic acid construct can comprise the sequence of

ATGGCCCTGCCTGTTACAGCTCTGCTGCTGCCTCTGGCTCTGCTTCTGC
ATGCCGCTAGACCTGAGATCGTGATGACACAGACCCCTGCCACACTGTC
TGTGTCTGCCGGCGAGAGAGTGACCATTACCTGCAAGGCCAGCCAGAGC
GTGTCCAACGACGTGACCTGGTATCAGCAGAAGCCAGGACAGGCCCCTC
GGCTGCTGATCTACAGCGCCAGCAATAGATACAGCGGCGTGCCCGCCAG
ATTTTCCGGCTCTGGATACGGCACCGAGTTCACCTTCACCATCAGCTCC
GTGCAGAGCGAGGACTTCGCTGTGTACTTCTGTCAGCAAGACTACAGCT
CCTTCGGCCAGGGCACCAAGCTGGAAATCAAGAGAGGAGGCGGAGGATC
TGGTGGCGGAGGAAGTGGCGGAGGCGGTTCTGGCGGTGGTGGATCTGAG
CAGAAGCTGATCTCCGAAGAGGACCTCCAGGTGCAGCTGGTGGAATCTG
GACCTGGTGTTGTGCAGCCTGGCAGAAGCCTGAGAATCAGCTGTGCCGT
GTCCGGCTTCAGCGTGACCAATTATGGCGTGCACTGGGTCCGACAGCCT
CCAGGCAAAGGACTGGAATGGCTGGGAGTGATTTGGGCTGGCGGCATCA
CCAACTACAACAGCGCCTTCATGAGCCGGCTGACCATCAGCAAGGACAA
CAGCAAGAACACCGTGTACCTGCAGATGAACAGCCTGAGAGCCGAGGAC
ACCGCCATGTACTACTGTGCTTCTAGAGGCGGCCACTACGGCTACGCCC
TGGATTATTGGGGACAGGGCACACTGGTCACAGTGTCTAGCCCTTGCGT
GGGCAGCAACCCCTGCTACAATCAGGGCACATGCGAGCCCACCAGCGAG
AACCCCTTCTACAGATGTCTGTGCCCCGCCAAGTTCAACGGCCTGCTGT
GTCACATCCTGGACTACAGCTTTACCGGCGGAGCCGGCAGAGATATCCC
TCCACCTCAGATTGAGGAAGCCTGCGAGCTGCCTGAGTGTCAGGTTGAC
GCCGGCAACAAAGTGTGCAACCTGCAGTGCAACAACCACGCCTGTGGAT
GGGATGGCGGCGACTGTAGCCTGAACTTCAACGACCCCTGGAAGAACTG
CACCCAGAGCCTGCAGTGTTGGAAGTACTTTAGCGACGGCCACTGCGAC
AGCCAGTGTAATTCTGCCGGATGCCTGTTCGACGGCTTCGACTGCCAAC
TGACAGAGGGCCAGTGCAACCCTCTGTACGACCAGTACTGCAAGGACCA
CTTCTCCGATGGCCACTGTGACCAGGGCTGTAATAGCGCCGAGTGCGAG
TGGGATGGACTGGATTGTGCCGAGCACGTGCCAGAAAGACTGGCCGCTG
GAACACTGGTGCTGGTGGTTCTTCTGCCTCCTGACCAGCTGCGGAACAA
CAGCTTCCACTTCCTGCGGGAACTGAGCCACGTGCTGCACACCAACGTG
GTGTTCAAGAGAGATGCCCAGGGACAGCAGATGATCTTCCCCTACTACG
GCCACGAAGAGGAACTGCGGAAGCACCCCATCAAGAGATCTACAGTCGG
CTGGGCCACCTCCAGTCTGCTGCCTGGAACAAGTGGCGGCAGACAGAGA
AGAGAACTGGACCCCATGGACATCCGGGGCAGCATCGTGTACCTGGAAA
TCGACAACCGGCAGTGCGTGCAGAGCAGCTCCCAGTGTTTTCAGAGCGC
TACTGACGTGGCCGCCTTTCTGGGAGCACTTGCTTCTCTGGGCAGCCTG
AACATCCCCTACAAGATCGAGGCCGTGAAGTCCGAGCCTGTGGAACCTC
CTCTGCCTTCTCAGCTGCACCTTATGTACGTGGCAGCCGCCGCTTTCGT
GCTGCTGTTCTTTGTTGGATGCGGAGTGCTGCTGAGCCGGAAGCGGAGA
AGAATGAAGCTGCTGTCCAGCATCGAGCAGGCCTGTGACATCTGCAGAC
TGAAGAAACTGAAGTGCAGCAAAGAAAAGCCCAAGTGCGCCAAGTGCCT
GAAGAACAATTGGGAGTGCCGGTACAGCCCCAAGACCAAGAGATCCCCT
CTGACAAGAGCCCACCTGACCGAGGTGGAAAGCCGGCTGGAAAGACTCG
AGCAGCTGTTCCTGCTGATCTTTCCACGCGAGGACCTGGACATGATTCT
GAAGATGGACTCTCTGCAGGACATCAAGGCCCTGCTGACCGGCCTGTTC
GTGCAGGACAACGTGAACAAGGACGCCGTGACCGATAGACTGGCCTCCG
TGGAAACCGACATGCCCCTGACACTGAGACAGCACAGAATCAGCGCCAC
CAGCAGCAGCGAGGAAAGCAGCAACAAGGGCCAGAGACAGCTGACAGTG
TCTGCTGCAGCTGGCGGATCAGGTGGTAGTGGCGGATCTGATGCCCTGG
ACGACTTTGACCTGGATATGCTGGGCAGCGACGCCCTGGATGATTTTGA
TCTGGACATGCTCGGCTCCGACGCTCTCGACGATTTCGACCTCGACATG
TTGGGATCCGACGCACTTGATGACTTCGATCTCGATATGCTCGGGTCCT
GA

or a variant thereof.

In some aspects, this sequence can be referred to as 3F8VH_VL_ERR-Gal4VP64. The bold sequence encodes a signal peptide. The italics sequence encodes the variable light (VL) domain. The underline sequence is a linker The double underlined sequence encodes the variable heavy (VH) domain. The italics and underlined sequence encodes the ERR. The bold and underlined sequence encodes the notch transmembrane domain. The bold, italics, and double underlined sequence encodes the Gal4-VP64 transcription activator.

In some aspects, a nucleic acid construct can comprise the sequence of

ATGGCCCTGCCTGTTACAGCTCTGCTGCTGCCTCTGGCTCTGCTT
CTGCATGCCGCTAGACCTGAGCAGAAGCTGATCTCCGAAGAGGAC
CTCCAGGTCCAGCTGCAAGAATCTGGCCCTGGCCTGGTCAAGCCT
AGCCAGACACTGAGCATCACCTGTACCGTGTCCGGCTTTAGCCTG
GCCAGCTACAACATCCACTGGGTCCGACAGCCTCCAGGCAAAGGA
CTGGAATGGCTGGGAGTGATTTGGGCTGGCGGCAGCACCAACTAC
AACAGCGCCCTGATGAGCCGGCTGACCATCAGCAAGGACAACAGC
AAGAACCAGGTGTTCCTGAAGATGAGCAGCCTGACAGCCGCCGAT
ACCGCCGTGTACTACTGTGCCAAGAGAAGCGACGACTACAGTTGG
TTCGCCTACTGGGGCCAGGGCACACTGGTTACAGTTTCTAGCGGA
GGCGGAGGATCTGGTGGCGGAGGAAGTGGCGGAGGCGGTTCTGAG
AATCAGATGACACAGAGCCCCAGCAGCCTGTCTGCCTCTGTGGGA
GATAGAGTGACCATGACCTGCAGAGCCTCCAGCTCCGTGTCTAGC
AGCTACCTGCACTGGTATCAGCAGAAGTCCGGCAAGGCCCCTAAA
GTGTGGATCTACAGCACCAGCAATCTGGCCAGCGGCGTGCCAAGC
AGATTTTCTGGAAGCGGCAGCGGCACCGACTACACCCTGACCATA
TCTAGCCTGCAGCCTGAGGACTTCGCCACCTACTACTGCCAGCAG
TACAGCGGCTACCCCATCACATTTGGACAGGGCACCAAGGTGGAA
ATCAAGAGGCCTTGCGTGGGCAGCAACCCCTGCTACAATCAGGGC
ACATGCGAGCCCACCAGCGAGAACCCCTTCTACAGATGTCTGTGC
CCCGCCAAGTTCAACGGCCTGCTGTGTCACATCCTGGACTACAGC
TTTACCGGCGGAGCCGGCAGAGATATCCCTCCACCTCAGATTGAG
GAAGCCTGCGAGCTGCCTGAGTGTCAGGTTGACGCCGGCAACAAA
GTGTGCAACCTGCAGTGCAACAACCACGCCTGTGGATGGGATGGC
GGCGACTGTAGCCTGAACTTCAACGACCCCTGGAAGAACTGCACC
CAGAGCCTGCAGTGTTGGAAGTACTTTAGCGACGGCCACTGCGAC
AGCCAGTGTAATTCTGCCGGATGCCTGTTCGACGGCTTCGACTGC
CAACTGACAGAGGGCCAGTGCAACCCTCTGTACGACCAGTACTGC
AAGGACCACTTCTCCGATGGCCACTGTGACCAGGGCTGTAATAGC
GCCGAGTGCGAGTGGGATGGACTGGATTGTGCCGAGCACGTGCCA
GAAAGACTGGCCGCTGGAACACTGGTGCTGGTGGTTCTTCTGCCT
CCTGACCAGCTGCGGAACAACAGCTTCCACTTCCTGCGGGAACTG
AGCCACGTGCTGCACACCAACGTGGTGTTCAAGAGAGATGCCCAG
GGACAGCAGATGATCTTCCCCTACTACGGCCACGAAGAGGAACTG
CGGAAGCACCCCATCAAGAGATCTACAGTCGGCTGGGCCACCTCC
AGTCTGCTGCCTGGAACAAGTGGCGGCAGACAGAGAAGAGAACTG
GACCCCATGGACATCCGGGGCAGCATCGTGTACCTGGAAATCGAC
AACCGGCAGTGCGTGCAGAGCAGCTCCCAGTGTTTTCAGAGCGCT
ACTGACGTGGCCGCCTTTCTGGGAGCACTTGCTTCTCTGGGCAGC
CTGAACATCCCCTACAAGATCGAGGCCGTGAAGTCCGAGCCTGTG
GAACCTCCTCTGCCTTCTCAGCTGCACCTTATGTACGTGGCAGCC
GCCGCTTTCGTGCTGCTGTTCTTTGTTGGATGCGGAGTGCTGCTG
AGCCGGAAGCGGAGAAGAATGAAGCTGCTGTCCAGCATCGAGCAG
GCCTGTGACATCTGCAGACTGAAGAAACTGAAGTGCAGCAAAGAA
AAGCCCAAGTGCGCCAAGTGCCTGAAGAACAATTGGGAGTGCCGG
TACAGCCCCAAGACCAAGAGATCCCCTCTGACAAGAGCCCACCTG
ACCGAGGTGGAAAGCCGGCTGGAAAGACTCGAGCAGCTGTTCCTG
CTGATCTTTCCACGCGAGGACCTGGACATGATTCTGAAGATGGAC
TCTCTGCAGGACATCAAGGCCCTGCTGACCGGCCTGTTCGTGCAG
GACAACGTGAACAAGGACGCCGTGACCGATAGACTGGCCTCCGTG
GAAACCGACATGCCCCTGACACTGAGACAGCACAGAATCAGCGCC
ACCAGCAGCAGCGAGGAAAGCAGCAACAAGGGCCAGAGACAGCTG
ACAGTGTCTGCTGCAGCTGGCGGATCAGGTGGTAGTGGCGGATCT
GATGCCCTGGACGACTTTGACCTGGATATGCTGGGCAGCGACGCC
CTGGATGATTTTGATCTGGACATGCTCGGCTCCGACGCTCTCGAC
GATTTCGACCTCGACATGTTGGGATCCGACGCACTTGATGACTTC
GATCTCGATATGCTCGGGTCCTGA,

or a variant thereof.

In some aspects, this sequence can be referred to as KM666-VHVL-ERR. The bold sequence encodes a signal peptide. The double underlined sequence encodes the VH domain. The underline sequence is a linker. The italics sequence encodes the VL domain. The italics and underlined sequence encodes the ERR. The bold and underlined sequence encodes the notch transmembrane domain. The bold, italics, and double underlined sequence encodes the Gal4-VP64 transcription activator.

In some aspects, a nucleic acid construct can comprise the sequence of

(SEQ ID NO: X)
CGATACCGTCGACCAAGGCAGCTGTAGATCTTAGCCACTTTTTAAAAGAAAAGGG
GGGACTGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTCA
GTGTGGAAAATCTCTAGCAGCATCTAGAATTAATTCCGTGTATTCTATAGTGTCA
CCTAAATCGTATGTGTATGATACATAAGGTTATGTATTAATTGTAGCCGCGTTCT
AACGACAATATGTACAAGCCTAATTGTGTAGCATCTGGCTTACTGAAGCAGACCC
TATCATCTCTCTCGTAAACTGCCGTCAGAGTCGGTTTGGTTGGACGAACCTTCTG
AGTTTCTGGTAACGCCGTCCCGCACCCGGAAATGGTCAGCGAACCAATCAGCAGG
GTCATCGCTAGCCAGATCCTCTACGCCGGACGCATCGTGGCCGGCATCACCGGCG
CCACAGGTGCGGTTGCTGGCGCCTATATCGCCGACATCACCGATGGGGAAGATCG
GGCTCGCCACTTCGGGCTCATGAGCGCTTGTTTCGGCGTGGGTATGGTGGCAGGC
CCCGTGGCCGGGGGACTGTTGGGCGCCATCTCCTTGCATGCACCATTCCTTGCGG
CGGCGGTGCTCAACGGCCTCAACCTACTACTGGGCTGCTTCCTAATGCAGGAGTC
GCATAAGGGAGAGCGTCGAATGGTGCACTCTCAGTACAATCTAGCTCTGATGCCG
CATAGTTAAGCCAGCCCCGACACCCGCCAACACCCGCTGACGCGCCCTGACGGGC
TTGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCTCG
TGATACGCCTATTTTTATAGGTTAATGTCATGATAATAATGGTTTCTTAGACGTC
AGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAAT
AATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCAG
TGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGAA
CTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAA
ATCTGGAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTA
ACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCT
GATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTA
ATGCAGCTGTGGAATGTGTGTCAGTTAGGGTGTGGAAAGTCCCCAGGCTCCCCAG
CAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTT
CCGCCCATTCTCCGCCCCATGGCTGACTAATTTTTTTTATTTATGCAGAGGCCGA
GGCCGCCTCGGCCTCTGAGCTATTCCAGAAGTAGTGAGGAGGCTTTTTTGGAGGC
CTAGGCTTTTGCAAAAAGCTTGGACACAAGACAGGCTTGCGAGATATGTTTGAGA
ATACCACTTTATCCCGCGTCAGGGAGAGGCAGTGCGTAAAAAGACGCGGACTCAT
GTGAAATACTGGTTTTTAGTGCGCCAGATCTCTATAATCTCGCGCAACCTATTTT
CCCCTCGAACACTTTTTAAGCCGTAGATAAACAGGCTGGGACACTTCACATGAGC
GAAAAATACATCGTCACCTGGGACATGTTGCAGATCCATGCACGTAAACTCGCAA
GCCGACTGATGCCTTCTGAACAATGGAAAGGCATTATTGCCGTAAGCCGTGGCGG
TCTGTACCGGGTGCGTTACTGGCGCGTGAACTGGGTATTCGTCATGTCGATACCG
TTTGTATTTCCAGCTACGATCACGACAACCAGCGCGAGCTTAAAGTGCTGAAACG
CGCAGAAGGCGATGGCGAAGGCTTCATCGTTATTGATGACCTGGTGGATACCGGT
GGTACTGCGGTTGCGATTCGTGAAATGTATCCAAAAGCGCACTTTGTCACCATCT
TCGCAAAACCGGCTGGTCGTCCGCTGGTTGATGACTATGTTGTTGATATCCCGCA
AGATACCTGGATTGAACAGCCGTGGGATATGGGCGTCGTATTCGTCCCGCCAATC
TCCGGTCGCTAATCTTTTCAACGCCTGGCACTGCCGGGCGTTGTTCTTTTTAACT
TCAGGCGGGTTACAATAGTTTCCAGTAAGTATTCTGGAGGCTGCATCCATGACAC
AGGCAAACCTGAGCGAAACCCTGTTCAAACCCCGCTTTAAACATCCTGAAACCTC
GACGCTAGTCCGCCGCTTTAATCACGGCGCACAACCGCCTGTGCAGTCGGCCCTT
GATGGTAAAACCATCCCTCACTGGTATCGCATGATTAACCGTCTGATGTGGATCT
GGCGCGGCATTGACCCACGCGAAATCCTCGACGTCCAGGCACGTATTGTGATGAG
CGATGCCGAACGTACCGACGATGATTTATACGATACGGTGATTGGCTACCGTGGC
GGCAACTGGATTTATGAGTGGGCCCCGGATCTTTGTGAAGGAACCTTACTTCTGT
GGTGTGACATAATTGGACAAACTACCTACAGAGATTTAAAGCTCTAAGGTAAATA
TAAAATTTTTAAGTGTATAATGTGTTAAACTACTGATTCTAATTGTTTGTGTATT
TTAGATTCCAACCTATGGAACTGATGAATGGGAGCAGTGGTGGAATGCCTTTAAT
GAGGAAAACCTGTTTTGCTCAGAAGAAATGCCATCTAGTGATGATGAGGCTACTG
CTGACTCTCAACATTCTACTCCTCCAAAAAAGAAGAGAAAGGTAGAAGACCCCAA
GGACTTTCCTTCAGAATTGCTAAGTTTTTTGAGTCATGCTGTGTTTAGTAATAGA
ACTCTTGCTTGCTTTGCTATTTACACCACAAAGGAAAAAGCTGCACTGCTATACA
AGAAAATTATGGAAAAATATTCTGTAACCTTTATAAGTAGGCATAACAGTTATAA
TCATAACATACTGTTTTTTCTTACTCCACACAGGCATAGAGTGTCTGCTATTAAT
AACTATGCTCAAAAATTGTGTACCTTTAGCTTTTTAATTTGTAAAGGGGTTAATA
AGGAATATTTGATGTATAGTGCCTTGACTAGAGATCATAATCAGCCATACCACAT
TTGTAGAGGTTTTACTTGCTTTAAAAAACCTCCCACACCTCCCCCTGAACCTGAA
ACATAAAATGAATGCAATTGTTGTTGTTAACTTGTTTATTGCAGCTTATAATGGT
TACAAATAAAGCAATAGCATCACAAATTTCACAAATAAAGCATTTTTTTCACTGC
ATTCTAGTTGTGGTTTGTCCAAACTCATCAATGTATCTTATCATGTCTGGATCAA
CTGGATAACTCAAGCTAACCAAAATCATCCCAAACTTCCCACCCCATACCCTATT
ACCACTGCCAATTACCTAGTGGTTTCATTTACTCTAAACCTGTGATTCCTCTGAA
TTATTTTCATTTTAAAGAAATTGTATTTGTTAAATATGTACTACAAACTTAGTAG
TTGGAAGGGCTAATTCACTCCCAAAGAAGACAAGATATCCTTGATCTGTGGATCT
ACCACACACAAGGCTACTTCCCTGATTAGCAGAACTACACACCAGGGCCAGGGGT
CAGATATCCACTGACCTTTGGATGGTGCTACAAGCTAGTACCAGTTGAGCCAGAT
AAGGTAGAAGAGGCCAATAAAGGAGAGAACACCAGCTTGTTACACCCTGTGAGCC
TGCATGGGATGGATGACCCGGAGAGAGAAGTGTTAGAGTGGAGGTTTGACAGCCG
CCTAGCATTTCATCACGTGGCCCGAGAGCTGCATCCGGAGTACTTCAAGAACTGC
TGATATCGAGCTTGCTACAAGGGACTTTCCGCTGGGGACTTTCCAGGGAGGCGTG
GCCTGGGCGGGACTGGGGAGTGGCGAGCCCTCAGATCCTGCATATAAGCAGCTGC
TTTTTGCCTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGAC
CCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAG
CGGAGGCTAGAAGGAGAGAGATGGGTGCGAGAGCGTCAGTATTAAGCGGGGGAGA
ATTAGATCGCGATGGGAAAAAATTCGGTTAAGGCCAGGGGGAAAGAAAAAATATA
AATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCA
TCCCTTCAGACAGGATCAGAAGAACTTAGATCATTATATAATACAGTAGCAACCC
TCTATTGTGTGCATCAAAGGATAGAGATAAAAGACACCAAGGAAGCTTTAGACAA
GATAGAGGAAGAGCAAAACAAAAGTAAGACCACCGCACAGCAAGCGGCCGGTGAT
CTTCAGACCTGGACGATATATATGAGGGACAATTGGAGAAGTGAATTATATAAAT
ATAAAGTAGTAAAAATTGAACCATTAGGAGTAGCACCCACCAAGGCAAAGAGAAG
AGTGGTGCAGAGAGAAAAAAGAGCAGTGGGAATAGGAGCTTTGTTCCTTGGGTTC
TTGGGAGCAGCAGGAAGCACTATGGGCGCAGCGTCAATGACGCTGACGGTACAGG
CCAGACAATTATTGTCTGGTATAGTGCAGCAGCAGAACAATTTGCTGAGGGCTAT
TGAGGCGCAACAGCATCTGTTGCAACTCACAGTCTGGGGCATCAAGCAGCTCCAG
GCAAGAATCCTGGCTGTGGAAAGATACCTAAAGGATCAACAGCTCCTGGGGATTT
GGGGTTGCTCTGGAAAACTCATTTGCACCACTGCTGTGCCTTGGAATGCTAGTTG
GAGTAATAAATCTCTGGAACAGATTTGGAATCACACGACCTGGATGGAGTGGGAC
AGAGAAATTAACAATTACACAAGCTTAATACACTCCTTAATTGAAGAATCGCAAA
ACCAGCAAGAAAAGAATGAACAAGAATTATTGGAATTAGATAAATGGGCAAGTTT
GTGGAATTGGTTTAACATAACAAATTGGCTGTGGTATATAAAATTATTCATAATG
ATAGTAGGAGGCTTGGTAGGTTTAAGAATAGTTTTTGCTGTACTTTCTATAGTGA
ATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGA
GACAGATCCATTCGATTAGTGAACGGATCTCGACGGTCGCCAAATGGCAGTATTC
ATCCACAATTTTAAAAGAAAAGGGGGGATTGGGGGGTACAGTGCAGGGGAAAGAA
TAGTAGACATAATAGCAACAGACATACAAACTAAAGAATTACAAAAACAAATTAC
AAAAATTCAAAATTTTCGGGTTTATTACAGGGACAGCAGAGATCCAGTTTGGATC
GATAAGCTTGATATCGAATTGGGTAGGGGAGGCGCTTTTCCCAAGGCAGTCTGGA
GCATGCGCTTTAGCAGCCCCGCTGGGCACTTGGCGCTACACAAGTGGCCTCTGGC
CTCGCACACATTCCACATCCACCGGTAGGCGCCAACCGGCTCCGTTCTTTGGTGG
CCCCTTCGCGCCACCTTCTACTCCTCCCCTAGTCAGGAAGTTCCCCCCCGCCCCG
CAGCTCGCGTCGTGCAGGACGTGACAAATGGAAGTAGCACGTCTCACTAGTCTCG
TGCAGATGGACAGCACCGCTGAGCAATGGAAGCGGGTAGGCCTTTGGGGCAGCGG
CCAATAGCAGCTTTGCTCCTTCGCTTTCTGGGCTCAGAGGCTGGGAAGGGGTGGG
TCCGGGGGCGGGCTCAGGGGGGGGCTCAGGGGCGGGGGGGGCGCCCGAAGGTCCT
CCGGAGGCCCGGCATTCTGCACGCTTCAAAAGCGCACGTCTGCCGCGCTGTTCTC
CTCTTCCTCATCTCCGGGCCTTTCGAATTCTCACGCGTCAAGTGGAGCAAGGCAG
GTGGACAGTGGATCATGGCCCTGCCTGTTACAGCTCTGCTGCTGCCTCTGGCTCT
GCTTCTGCATGCCGCTAGACCTGAGATCGTGATGACACAGACCCCTGCCACACTG
TCTGTGTCTGCCGGCGAGAGAGTGACCATTACCTGCAAGGCCAGCCAGAGCGTGT
CCAACGACGTGACCTGGTATCAGCAGAAGCCAGGACAGGCCCCTCGGCTGCTGAT
CTACAGCGCCAGCAATAGATACAGCGGCGTGCCCGCCAGATTTTCCGGCTCTGGA
TACGGCACCGAGTTCACCTTCACCATCAGCTCCGTGCAGAGCGAGGACTTCGCTG
TGTACTTCTGTCAGCAAGACTACAGCTCCTTCGGCCAGGGCACCAAGCTGGAAAT
CAAGAGAGGAGGCGGAGGATCTGGTGGCGGAGGAAGTGGCGGAGGCGGTTCTGGC
GGTGGTGGATCTGAGCAGAAGCTGATCTCCGAAGAGGACCTCCAGGTGCAGCTGG
TGGAATCTGGACCTGGTGTTGTGCAGCCTGGCAGAAGCCTGAGAATCAGCTGTGC
CGTGTCCGGCTTCAGCGTGACCAATTATGGCGTGCACTGGGTCCGACAGCCTCCA
GGCAAAGGACTGGAATGGCTGGGAGTGATTTGGGCTGGCGGCATCACCAACTACA
ACAGCGCCTTCATGAGCCGGCTGACCATCAGCAAGGACAACAGCAAGAACACCGT
VGTACCTGCAGATGAACAGCCTGAGAGCCGAGGACACCGCCATGTACTACTGTGCT
TCTAGAGGCGGCCACTACGGCTACGCCCTGGATTATTGGGGACAGGGCACACTGG
TCACAGTGTCTAGCCCTTGCGTGGGCAGCAACCCCTGCTACAATCAGGGCACATG
CGAGCCCACCAGCGAGAACCCCTTCTACAGATGTCTGTGCCCCGCCAAGTTCAAC
GGCCTGCTGTGTCACATCCTGGACTACAGCTTTACCGGCGGAGCCGGCAGAGATA
TCCCTCCACCTCAGATTGAGGAAGCCTGCGAGCTGCCTGAGTGTCAGGTTGACGC
CGGCAACAAAGTGTGCAACCTGCAGTGCAACAACCACGCCTGTGGATGGGATGGC
GGCGACTGTAGCCTGAACTTCAACGACCCCTGGAAGAACTGCACCCAGAGCCTGC
AGTGTTGGAAGTACTTTAGCGACGGCCACTGCGACAGCCAGTGTAATTCTGCCGG
ATGCCTGTTCGACGGCTTCGACTGCCAACTGACAGAGGGCCAGTGCAACCCTCTG
TACGACCAGTACTGCAAGGACCACTTCTCCGATGGCCACTGTGACCAGGGCTGTA
ATAGCGCCGAGTGCGAGTGGGATGGACTGGATTGTGCCGAGCACGTGCCAGAAAG
ACTGGCCGCTGGAACACTGGTGCTGGTGGTTCTTCTGCCTCCTGACCAGCTGCGG
AACAACAGCTTCCACTTCCTGCGGGAACTGAGCCACGTGCTGCACACCAACGTGG
TGTTCAAGAGAGATGCCCAGGGACAGCAGATGATCTTCCCCTACTACGGCCACGA
AGAGGAACTGCGGAAGCACCCCATCAAGAGATCTACAGTCGGCTGGGCCACCTCC
AGTCTGCTGCCTGGAACAAGTGGCGGCAGACAGAGAAGAGAACTGGACCCCATGG
ACATCCGGGGCAGCATCGTGTACCTGGAAATCGACAACCGGCAGTGCGTGCAGAG
CAGCTCCCAGTGTTTTCAGAGCGCTACTGACGTGGCCGCCTTTCTGGGAGCACTT
GCTTCTCTGGGCAGCCTGAACATCCCCTACAAGATCGAGGCCGTGAAGTCCGAGC
CTGTGGAACCTCCTCTGCCTTCTCAGCTGCACCTTATGTACGTGGCAGCCGCCGC
TTTCGTGCTGCTGTTCTTTGTTGGATGCGGAGTGCTGCTGAGCCGGAAGCGGAGA
AGAATGAAGCTGCTGTCCAGCATCGAGCAGGCCTGTGACATCTGCAGACTGAAGA
AACTGAAGTGCAGCAAAGAAAAGCCCAAGTGCGCCAAGTGCCTGAAGAACAATTG
GGAGTGCCGGTACAGCCCCAAGACCAAGAGATCCCCTCTGACAAGAGCCCACCTG
ACCGAGGTGGAAAGCCGGCTGGAAAGACTCGAGCAGCTGTTCCTGCTGATCTTTC
CACGCGAGGACCTGGACATGATTCTGAAGATGGACTCTCTGCAGGACATCAAGGC
CCTGCTGACCGGCCTGTTCGTGCAGGACAACGTGAACAAGGACGCCGTGACCGAT
AGACTGGCCTCCGTGGAAACCGACATGCCCCTGACACTGAGACAGCACAGAATCA
GCGCCACCAGCAGCAGCGAGGAAAGCAGCAACAAGGGCCAGAGACAGCTGACAGT
GTCTGCTGCAGCTGGCGGATCAGGTGGTAGTGGCGGATCTGATGCCCTGGACGAC
TTTGACCTGGATATGCTGGGCAGCGACGCCCTGGATGATTTTGATCTGGACATGC
TCGGCTCCGACGCTCTCGACGATTTCGACCTCGACATGTTGGGATCCGACGCACT
TGATGACTTCGATCTCGATATGCTCGGGTCCTGAGATCCTTGACTTGCGGCCGCA
ACTCCCACCTGCAACATGCGTGACTGACTGAGGCCGCGACTCTAGAGTCGACCTG
CAGGCATGCAAGCTTGATATCAAGCTTATCGATAATCAACCTCTGGATTACAAAA
TTTGTGAAAGATTGACTGGTATTCTTAACTATGTTGCTCCTTTTACGCTATGTGG
ATACGCTGCTTTAATGCCTTTGTATCATGCTATTGCTTCCCGTATGGCTTTCATT
TTCTCCTCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCCCACTGG
TTGGGGCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAATCATCGTCCTT
TCCTTGGCTGCTCGCCTGTGTTGCCACCTGGATTCTGCGCGGGACGTCCTTCTGC
TACGTCCCTTCGGCCCTCAATCCAGCGGACCTTCCTTCCCGCGGCCTGCTGCCGG
CTCTGCGGCCTCTTCCGCGTCTTCGCCTTCGCCCTCAGACGAGTCGGATCTCCCT
TTGGGCCGCCTCCCCGCAT,

or a variant thereof.

This is an example of a complete plasmid sequence for 3F8_VLVH. The single underlined sequence represents the VL sequence. The double underlined sequence represents the VH sequence.

In some aspects, a nucleic acid construct can comprise the sequence of

(SEQ ID NO: XX)
CGATACCGTCGACCAAGGCAGCTGTAGATCTTAGCCACTTTTTAAAAGAAAAGGG
GGGACTGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTCA
GTGTGGAAAATCTCTAGCAGCATCTAGAATTAATTCCGTGTATTCTATAGTGTCA
CCTAAATCGTATGTGTATGATACATAAGGTTATGTATTAATTGTAGCCGCGTTCT
AACGACAATATGTACAAGCCTAATTGTGTAGCATCTGGCTTACTGAAGCAGACCC
TATCATCTCTCTCGTAAACTGCCGTCAGAGTCGGTTTGGTTGGACGAACCTTCTG
AGTTTCTGGTAACGCCGTCCCGCACCCGGAAATGGTCAGCGAACCAATCAGCAGG
GTCATCGCTAGCCAGATCCTCTACGCCGGACGCATCGTGGCCGGCATCACCGGCG
CCACAGGTGCGGTTGCTGGCGCCTATATCGCCGACATCACCGATGGGGAAGATCG
GGCTCGCCACTTCGGGCTCATGAGCGCTTGTTTCGGCGTGGGTATGGTGGCAGGC
CCCGTGGCCGGGGGACTGTTGGGCGCCATCTCCTTGCATGCACCATTCCTTGCGG
CGGCGGTGCTCAACGGCCTCAACCTACTACTGGGCTGCTTCCTAATGCAGGAGTC
GCATAAGGGAGAGCGTCGAATGGTGCACTCTCAGTACAATCTAGCTCTGATGCCG
CATAGTTAAGCCAGCCCCGACACCCGCCAACACCCGCTGACGCGCCCTGACGGGC
TTGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCTCG
TGATACGCCTATTTTTATAGGTTAATGTCATGATAATAATGGTTTCTTAGACGTC
AGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAAT
AATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCAG
TGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGAA
CTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAA
ATCTGGAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTA
ACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCT
GATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTA
ATGCAGCTGTGGAATGTGTGTCAGTTAGGGTGTGGAAAGTCCCCAGGCTCCCCAG
CAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTT
CCGCCCATTCTCCGCCCCATGGCTGACTAATTTTTTTTATTTATGCAGAGGCCGA
GGCCGCCTCGGCCTCTGAGCTATTCCAGAAGTAGTGAGGAGGCTTTTTTGGAGGC
CTAGGCTTTTGCAAAAAGCTTGGACACAAGACAGGCTTGCGAGATATGTTTGAGA
ATACCACTTTATCCCGCGTCAGGGAGAGGCAGTGCGTAAAAAGACGCGGACTCAT
GTGAAATACTGGTTTTTAGTGCGCCAGATCTCTATAATCTCGCGCAACCTATTTT
CCCCTCGAACACTTTTTAAGCCGTAGATAAACAGGCTGGGACACTTCACATGAGC
GAAAAATACATCGTCACCTGGGACATGTTGCAGATCCATGCACGTAAACTCGCAA
GCCGACTGATGCCTTCTGAACAATGGAAAGGCATTATTGCCGTAAGCCGTGGCGG
TCTGTACCGGGTGCGTTACTGGCGCGTGAACTGGGTATTCGTCATGTCGATACCG
TTTGTATTTCCAGCTACGATCACGACAACCAGCGCGAGCTTAAAGTGCTGAAACG
CGCAGAAGGCGATGGCGAAGGCTTCATCGTTATTGATGACCTGGTGGATACCGGT
GGTACTGCGGTTGCGATTCGTGAAATGTATCCAAAAGCGCACTTTGTCACCATCT
TCGCAAAACCGGCTGGTCGTCCGCTGGTTGATGACTATGTTGTTGATATCCCGCA
AGATACCTGGATTGAACAGCCGTGGGATATGGGCGTCGTATTCGTCCCGCCAATC
TCCGGTCGCTAATCTTTTCAACGCCTGGCACTGCCGGGCGTTGTTCTTTTTAACT
TCAGGCGGGTTACAATAGTTTCCAGTAAGTATTCTGGAGGCTGCATCCATGACAC
AGGCAAACCTGAGCGAAACCCTGTTCAAACCCCGCTTTAAACATCCTGAAACCTC
GACGCTAGTCCGCCGCTTTAATCACGGCGCACAACCGCCTGTGCAGTCGGCCCTT
GATGGTAAAACCATCCCTCACTGGTATCGCATGATTAACCGTCTGATGTGGATCT
GGCGCGGCATTGACCCACGCGAAATCCTCGACGTCCAGGCACGTATTGTGATGAG
CGATGCCGAACGTACCGACGATGATTTATACGATACGGTGATTGGCTACCGTGGC
GGCAACTGGATTTATGAGTGGGCCCCGGATCTTTGTGAAGGAACCTTACTTCTGT
GGTGTGACATAATTGGACAAACTACCTACAGAGATTTAAAGCTCTAAGGTAAATA
TAAAATTTTTAAGTGTATAATGTGTTAAACTACTGATTCTAATTGTTTGTGTATT
TTAGATTCCAACCTATGGAACTGATGAATGGGAGCAGTGGTGGAATGCCTTTAAT
GAGGAAAACCTGTTTTGCTCAGAAGAAATGCCATCTAGTGATGATGAGGCTACTG
CTGACTCTCAACATTCTACTCCTCCAAAAAAGAAGAGAAAGGTAGAAGACCCCAA
GGACTTTCCTTCAGAATTGCTAAGTTTTTTGAGTCATGCTGTGTTTAGTAATAGA
ACTCTTGCTTGCTTTGCTATTTACACCACAAAGGAAAAAGCTGCACTGCTATACA
AGAAAATTATGGAAAAATATTCTGTAACCTTTATAAGTAGGCATAACAGTTATAA
TCATAACATACTGTTTTTTCTTACTCCACACAGGCATAGAGTGTCTGCTATTAAT
AACTATGCTCAAAAATTGTGTACCTTTAGCTTTTTAATTTGTAAAGGGGTTAATA
AGGAATATTTGATGTATAGTGCCTTGACTAGAGATCATAATCAGCCATACCACAT
TTGTAGAGGTTTTACTTGCTTTAAAAAACCTCCCACACCTCCCCCTGAACCTGAA
ACATAAAATGAATGCAATTGTTGTTGTTAACTTGTTTATTGCAGCTTATAATGGT
TACAAATAAAGCAATAGCATCACAAATTTCACAAATAAAGCATTTTTTTCACTGC
ATTCTAGTTGTGGTTTGTCCAAACTCATCAATGTATCTTATCATGTCTGGATCAA
CTGGATAACTCAAGCTAACCAAAATCATCCCAAACTTCCCACCCCATACCCTATT
ACCACTGCCAATTACCTAGTGGTTTCATTTACTCTAAACCTGTGATTCCTCTGAA
TTATTTTCATTTTAAAGAAATTGTATTTGTTAAATATGTACTACAAACTTAGTAG
TTGGAAGGGCTAATTCACTCCCAAAGAAGACAAGATATCCTTGATCTGTGGATCT
ACCACACACAAGGCTACTTCCCTGATTAGCAGAACTACACACCAGGGCCAGGGGT
CAGATATCCACTGACCTTTGGATGGTGCTACAAGCTAGTACCAGTTGAGCCAGAT
AAGGTAGAAGAGGCCAATAAAGGAGAGAACACCAGCTTGTTACACCCTGTGAGCC
TGCATGGGATGGATGACCCGGAGAGAGAAGTGTTAGAGTGGAGGTTTGACAGCCG
CCTAGCATTTCATCACGTGGCCCGAGAGCTGCATCCGGAGTACTTCAAGAACTGC
TGATATCGAGCTTGCTACAAGGGACTTTCCGCTGGGGACTTTCCAGGGAGGCGTG
GCCTGGGCGGGACTGGGGAGTGGCGAGCCCTCAGATCCTGCATATAAGCAGCTGC
TTTTTGCCTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGAC
CCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAG
CGGAGGCTAGAAGGAGAGAGATGGGTGCGAGAGCGTCAGTATTAAGCGGGGGAGA
ATTAGATCGCGATGGGAAAAAATTCGGTTAAGGCCAGGGGGAAAGAAAAAATATA
AATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCA
TCCCTTCAGACAGGATCAGAAGAACTTAGATCATTATATAATACAGTAGCAACCC
TCTATTGTGTGCATCAAAGGATAGAGATAAAAGACACCAAGGAAGCTTTAGACAA
GATAGAGGAAGAGCAAAACAAAAGTAAGACCACCGCACAGCAAGCGGCCGGTGAT
CTTCAGACCTGGACGATATATATGAGGGACAATTGGAGAAGTGAATTATATAAAT
ATAAAGTAGTAAAAATTGAACCATTAGGAGTAGCACCCACCAAGGCAAAGAGAAG
AGTGGTGCAGAGAGAAAAAAGAGCAGTGGGAATAGGAGCTTTGTTCCTTGGGTTC
TTGGGAGCAGCAGGAAGCACTATGGGCGCAGCGTCAATGACGCTGACGGTACAGG
CCAGACAATTATTGTCTGGTATAGTGCAGCAGCAGAACAATTTGCTGAGGGCTAT
TGAGGCGCAACAGCATCTGTTGCAACTCACAGTCTGGGGCATCAAGCAGCTCCAG
GCAAGAATCCTGGCTGTGGAAAGATACCTAAAGGATCAACAGCTCCTGGGGATTT
GGGGTTGCTCTGGAAAACTCATTTGCACCACTGCTGTGCCTTGGAATGCTAGTTG
GAGTAATAAATCTCTGGAACAGATTTGGAATCACACGACCTGGATGGAGTGGGAC
AGAGAAATTAACAATTACACAAGCTTAATACACTCCTTAATTGAAGAATCGCAAA
ACCAGCAAGAAAAGAATGAACAAGAATTATTGGAATTAGATAAATGGGCAAGTTT
GTGGAATTGGTTTAACATAACAAATTGGCTGTGGTATATAAAATTATTCATAATG
ATAGTAGGAGGCTTGGTAGGTTTAAGAATAGTTTTTGCTGTACTTTCTATAGTGA
ATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGA
GACAGATCCATTCGATTAGTGAACGGATCTCGACGGTCGCCAAATGGCAGTATTC
ATCCACAATTTTAAAAGAAAAGGGGGGATTGGGGGGTACAGTGCAGGGGAAAGAA
TAGTAGACATAATAGCAACAGACATACAAACTAAAGAATTACAAAAACAAATTAC
AAAAATTCAAAATTTTCGGGTTTATTACAGGGACAGCAGAGATCCAGTTTGGATC
GATAAGCTTGATATCGAATTGGGTAGGGGAGGCGCTTTTCCCAAGGCAGTCTGGA
GCATGCGCTTTAGCAGCCCCGCTGGGCACTTGGCGCTACACAAGTGGCCTCTGGC
CTCGCACACATTCCACATCCACCGGTAGGCGCCAACCGGCTCCGTTCTTTGGTGG
CCCCTTCGCGCCACCTTCTACTCCTCCCCTAGTCAGGAAGTTCCCCCCCGCCCCG
CAGCTCGCGTCGTGCAGGACGTGACAAATGGAAGTAGCACGTCTCACTAGTCTCG
TGCAGATGGACAGCACCGCTGAGCAATGGAAGCGGGTAGGCCTTTGGGGCAGCGG
CCAATAGCAGCTTTGCTCCTTCGCTTTCTGGGCTCAGAGGCTGGGAAGGGGTGGG
TCCGGGGGCGGGCTCAGGGGGGGGCTCAGGGGCGGGGGGGGCGCCCGAAGGTCCT
CCGGAGGCCCGGCATTCTGCACGCTTCAAAAGCGCACGTCTGCCGCGCTGTTCTC
CTCTTCCTCATCTCCGGGCCTTTCGAATTCTCACGCGTCAAGTGGAGCAAGGCAG
GTGGACAGTGGATCATGGCCCTGCCTGTTACAGCTCTGCTGCTGCCTCTGGCTCT
GCTTCTGCATGCCGCTAGACCTGAGCAGAAGCTGATCTCCGAAGAGGACCTCCAG
GTCCAGCTGCAAGAATCTGGCCCTGGCCTGGTCAAGCCTAGCCAGACACTGAGCA
TCACCTGTACCGTGTCCGGCTTTAGCCTGGCCAGCTACAACATCCACTGGGTCCG
ACAGCCTCCAGGCAAAGGACTGGAATGGCTGGGAGTGATTTGGGCTGGCGGCAGC
ACCAACTACAACAGCGCCCTGATGAGCCGGCTGACCATCAGCAAGGACAACAGCA
AGAACCAGGTGTTCCTGAAGATGAGCAGCCTGACAGCCGCCGATACCGCCGTGTA
CTACTGTGCCAAGAGAAGCGACGACTACAGTTGGTTCGCCTACTGGGGCCAGGGC
ACACTGGTTACAGTTTCTAGCGGAGGCGGAGGATCTGGTGGCGGAGGAAGTGGCG
GAGGCGGTTCTGAGAATCAGATGACACAGAGCCCCAGCAGCCTGTCTGCCTCTGT
GGGAGATAGAGTGACCATGACCTGCAGAGCCTCCAGCTCCGTGTCTAGCAGCTAC
CTGCACTGGTATCAGCAGAAGTCCGGCAAGGCCCCTAAAGTGTGGATCTACAGCA
CCAGCAATCTGGCCAGCGGCGTGCCAAGCAGATTTTCTGGAAGCGGCAGCGGCAC
CGACTACACCCTGACCATATCTAGCCTGCAGCCTGAGGACTTCGCCACCTACTAC
TGCCAGCAGTACAGCGGCTACCCCATCACATTTGGACAGGGCACCAAGGTGGAAA
TCAAGAGGCCTTGCGTGGGCAGCAACCCCTGCTACAATCAGGGCACATGCGAGCC
CACCAGCGAGAACCCCTTCTACAGATGTCTGTGCCCCGCCAAGTTCAACGGCCTG
CTGTGTCACATCCTGGACTACAGCTTTACCGGCGGAGCCGGCAGAGATATCCCTC
CACCTCAGATTGAGGAAGCCTGCGAGCTGCCTGAGTGTCAGGTTGACGCCGGCAA
CAAAGTGTGCAACCTGCAGTGCAACAACCACGCCTGTGGATGGGATGGCGGCGAC
TGTAGCCTGAACTTCAACGACCCCTGGAAGAACTGCACCCAGAGCCTGCAGTGTT
GGAAGTACTTTAGCGACGGCCACTGCGACAGCCAGTGTAATTCTGCCGGATGCCT
GTTCGACGGCTTCGACTGCCAACTGACAGAGGGCCAGTGCAACCCTCTGTACGAC
CAGTACTGCAAGGACCACTTCTCCGATGGCCACTGTGACCAGGGCTGTAATAGCG
CCGAGTGCGAGTGGGATGGACTGGATTGTGCCGAGCACGTGCCAGAAAGACTGGC
CGCTGGAACACTGGTGCTGGTGGTTCTTCTGCCTCCTGACCAGCTGCGGAACAAC
AGCTTCCACTTCCTGCGGGAACTGAGCCACGTGCTGCACACCAACGTGGTGTTCA
AGAGAGATGCCCAGGGACAGCAGATGATCTTCCCCTACTACGGCCACGAAGAGGA
ACTGCGGAAGCACCCCATCAAGAGATCTACAGTCGGCTGGGCCACCTCCAGTCTG
CTGCCTGGAACAAGTGGCGGCAGACAGAGAAGAGAACTGGACCCCATGGACATCC
GGGGCAGCATCGTGTACCTGGAAATCGACAACCGGCAGTGCGTGCAGAGCAGCTC
CCAGTGTTTTCAGAGCGCTACTGACGTGGCCGCCTTTCTGGGAGCACTTGCTTCT
CTGGGCAGCCTGAACATCCCCTACAAGATCGAGGCCGTGAAGTCCGAGCCTGTGG
AACCTCCTCTGCCTTCTCAGCTGCACCTTATGTACGTGGCAGCCGCCGCTTTCGT
GCTGCTGTTCTTTGTTGGATGCGGAGTGCTGCTGAGCCGGAAGCGGAGAAGAATG
AAGCTGCTGTCCAGCATCGAGCAGGCCTGTGACATCTGCAGACTGAAGAAACTGA
AGTGCAGCAAAGAAAAGCCCAAGTGCGCCAAGTGCCTGAAGAACAATTGGGAGTG
CCGGTACAGCCCCAAGACCAAGAGATCCCCTCTGACAAGAGCCCACCTGACCGAG
GTGGAAAGCCGGCTGGAAAGACTCGAGCAGCTGTTCCTGCTGATCTTTCCACGCG
AGGACCTGGACATGATTCTGAAGATGGACTCTCTGCAGGACATCAAGGCCCTGCT
GACCGGCCTGTTCGTGCAGGACAACGTGAACAAGGACGCCGTGACCGATAGACTG
GCCTCCGTGGAAACCGACATGCCCCTGACACTGAGACAGCACAGAATCAGCGCCA
CCAGCAGCAGCGAGGAAAGCAGCAACAAGGGCCAGAGACAGCTGACAGTGTCTGC
TGCAGCTGGCGGATCAGGTGGTAGTGGCGGATCTGATGCCCTGGACGACTTTGAC
CTGGATATGCTGGGCAGCGACGCCCTGGATGATTTTGATCTGGACATGCTCGGCT
CCGACGCTCTCGACGATTTCGACCTCGACATGTTGGGATCCGACGCACTTGATGA
CTTCGATCTCGATATGCTCGGGTCCTGAGATCCTTGACTTGCGGCCGCAACTCCC
ACCTGCAACATGCGTGACTGACTGAGGCCGCGACTCTAGAGTCGACCTGCAGGCA
TGCAAGCTTGATATCAAGCTTATCGATAATCAACCTCTGGATTACAAAATTTGTG
AAAGATTGACTGGTATTCTTAACTATGTTGCTCCTTTTACGCTATGTGGATACGC
TGCTTTAATGCCTTTGTATCATGCTATTGCTTCCCGTATGGCTTTCATTTTCTCC
TCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATT
GCCACGGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTTCCTTG
GCTGCTCGCCTGTGTTGCCACCTGGATTCTGCGCGGGACGTCCTTCTGCTACGTC
CCTTCGGCCCTCAATCCAGCGGACCTTCCTTCCCGCGGCCTGCTGCCGGCTCTGC
GGCCTCTTCCGCGTCTTCGCCTTCGCCCTCAGACGAGTCGGATCTCCCTTTGGGC
CGCCTCCCCGCAT,

or a variant thereof.

This is an example of a complete plasmid sequence for KM666. The single underlined sequence represents the VL sequence. The double underlined region represents the VH sequence.

### 2. SynNotch Response Constructs

Disclosed are nucleic acid constructs comprising an upstream activation sequence (UAS), promoter controlled by the UAS, and a nucleic acid sequence encoding IFNγ. In some aspects, the nucleic acid sequence encoding IFNγ is operably linked to the promoter.

In some aspects, the UAS is a Gal4-UAS. In some aspects, a Gal4-UAS can comprise the sequence GALSSERRSTVLRTSEHCPPNVGALSSE.

In some aspects, the UAS is upstream of the promoter in the disclosed nucleic acid constructs.

In some aspects, the promoter controlled by the UAS can be any known promoters or any of the promoters described herein. In some aspects, the promoter is a CMV promoter. In some aspects, the promoter is not active without a transcription factor binding to the UAS.

In some aspects, a nucleic acid sequence encoding IFNγ comprises the sequence

ATGAAATACACTTCCTATATACTCGCTTTTCAACTGTGCATCGTG
CTTGGTAGCTTGGGCTGCTATTGTCAGGACCCCTATGTGAAAGAA
GCTGAGAACCTTAAGAAGTATTTTAATGCTGGTCACTCTGACGTG
GCGGACAATGGGACATTGTTCCTGGGTATTTTGAAGAATTGGAAG
GAAGAATCAGATAGAAAAATAATGCAGTCACAGATCGTGTCCTTC
TACTTCAAACTTTTCAAAAATTTCAAGGACGACCAGTCCATTCAG
AAGTCAGTTGAAACAATCAAGGAAGACATGAACGTGAAATTTTTC
AATAGCAATAAAAAGAAAAGGGATGATTTTGAGAAGTTGACAAAT
TACTCCGTGACTGACCTCAACGTCCAAAGAAAAGCTATACACGAG
TTGATCCAAGTTATGGCCGAGTTGAGTCCGGCGGCGAAAACAGGA
AAACGAAAGAGATCCCAAATGCTGTTTAGAGGCCGCCGCGCAAGT
CAG,

or a variant thereof.

In some aspects, the IFNγ comprises the amino acid sequence

MKYTSYILAFQLCIVLGSLGCYCQDPYVKEAENLKKYFNAGHSDV
ADNGTLFLGILKNWKEESDRKIMQSQIVSFYFKLFKNFKDDQSIQ
KSVETIKEDMNVKFFNSNKKKRDDFEKLTNYSVTDLNVQRKAIHE
LIQVMAELSPAAKTGKRKRSQMLFRGRRASQ,

or a variant thereof.

In some aspects, the nucleic acid constructs comprising an upstream activation sequence (UAS), promoter controlled by the UAS, and a nucleic acid sequence encoding IFNγ can further comprise an IRES sequence and/or a nucleic acid sequence encoding a detection agent. A nucleic acid sequence encoding a detection agent can be any sequence that encodes an amino acid sequence used to detect the construct. For example, a detection agent can be a fluorescent protein, an enzyme that provides a color based reaction, or a small protein that can easily be detected such as a histidine tag. In some aspects, the presence of a detection agent allows for visual detection or purification of the disclosed nucleic acid constructs or the products thereof. For example, a detection agent can be, but is not limited to, a myc tag, his tag, fluorescent tag, FLAG tag, or hemagglutinin tag. In some aspects, the detection agent can be Mcherry.

An example construct comprising a Gal4 UAS, IFNγ, IRES, and mCherry is shown as follows:

CGATACCGTCGACCAAGGCAGCTGTAGATCTTAGCCACTTTTTAAAAGAAAAGGG
GGGACTGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTCA
GTGTGGAAAATCTCTAGCAGCATCTAGAATTAATTCCGTGTATTCTATAGTGTCA
CCTAAATCGTATGTGTATGATACATAAGGTTATGTATTAATTGTAGCCGCGTTCT
AACGACAATATGTACAAGCCTAATTGTGTAGCATCTGGCTTACTGAAGCAGACCC
TATCATCTCTCTCGTAAACTGCCGTCAGAGTCGGTTTGGTTGGACGAACCTTCTG
AGTTTCTGGTAACGCCGTCCCGCACCCGGAAATGGTCAGCGAACCAATCAGCAGG
GTCATCGCTAGCCAGATCCTCTACGCCGGACGCATCGTGGCCGGCATCACCGGCG
CCACAGGTGCGGTTGCTGGCGCCTATATCGCCGACATCACCGATGGGGAAGATCG
GGCTCGCCACTTCGGGCTCATGAGCGCTTGTTTCGGCGTGGGTATGGTGGCAGGC
CCCGTGGCCGGGGGACTGTTGGGCGCCATCTCCTTGCATGCACCATTCCTTGCGG
CGGCGGTGCTCAACGGCCTCAACCTACTACTGGGCTGCTTCCTAATGCAGGAGTC
GCATAAGGGAGAGCGTCGAATGGTGCACTCTCAGTACAATCTAGCTCTGATGCCG
CATAGTTAAGCCAGCCCCGACACCCGCCAACACCCGCTGACGCGCCCTGACGGGC
TTGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCTCG
TGATACGCCTATTTTTATAGGTTAATGTCATGATAATAATGGTTTCTTAGACGTC
AGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAAT
AATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACT
CACCAGTCACAGAAAAGCATCTTACGGATGGCATGACAGTAAGAGAATTATGCAG
TGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGA
CACCACGATGCCTGTAGCAATGGCAACAACGTTGCGCAAACTATTAACTGGCGAA
CTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAA
ATCTGGAGCCGGTGAGCGTGGGTCTCGCGGTATCATTGCAGCACTGGGGCCAGAT
GGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTA
ACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAA
CCACCGCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCT
GATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTA
ATGCAGCTGTGGAATGTGTGTCAGTTAGGGTGTGGAAAGTCCCCAGGCTCCCCAG
CAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTT
CCGCCCATTCTCCGCCCCATGGCTGACTAATTTTTTTTATTTATGCAGAGGCCGA
GGCCGCCTCGGCCTCTGAGCTATTCCAGAAGTAGTGAGGAGGCTTTTTTGGAGGC
CTAGGCTTTTGCAAAAAGCTTGGACACAAGACAGGCTTGCGAGATATGTTTGAGA
ATACCACTTTATCCCGCGTCAGGGAGAGGCAGTGCGTAAAAAGACGCGGACTCAT
GTGAAATACTGGTTTTTAGTGCGCCAGATCTCTATAATCTCGCGCAACCTATTTT
CCCCTCGAACACTTTTTAAGCCGTAGATAAACAGGCTGGGACACTTCACATGAGC
GAAAAATACATCGTCACCTGGGACATGTTGCAGATCCATGCACGTAAACTCGCAA
GCCGACTGATGCCTTCTGAACAATGGAAAGGCATTATTGCCGTAAGCCGTGGCGG
TCTGTACCGGGTGCGTTACTGGCGCGTGAACTGGGTATTCGTCATGTCGATACCG
TTTGTATTTCCAGCTACGATCACGACAACCAGCGCGAGCTTAAAGTGCTGAAACG
CGCAGAAGGCGATGGCGAAGGCTTCATCGTTATTGATGACCTGGTGGATACCGGT
GGTACTGCGGTTGCGATTCGTGAAATGTATCCAAAAGCGCACTTTGTCACCATCT
TCGCAAAACCGGCTGGTCGTCCGCTGGTTGATGACTATGTTGTTGATATCCCGCA
AGATACCTGGATTGAACAGCCGTGGGATATGGGCGTCGTATTCGTCCCGCCAATC
TCCGGTCGCTAATCTTTTCAACGCCTGGCACTGCCGGGCGTTGTTCTTTTTAACT
TCAGGCGGGTTACAATAGTTTCCAGTAAGTATTCTGGAGGCTGCATCCATGACAC
AGGCAAACCTGAGCGAAACCCTGTTCAAACCCCGCTTTAAACATCCTGAAACCTC
GACGCTAGTCCGCCGCTTTAATCACGGCGCACAACCGCCTGTGCAGTCGGCCCTT
GATGGTAAAACCATCCCTCACTGGTATCGCATGATTAACCGTCTGATGTGGATCT
GGCGCGGCATTGACCCACGCGAAATCCTCGACGTCCAGGCACGTATTGTGATGAG
CGATGCCGAACGTACCGACGATGATTTATACGATACGGTGATTGGCTACCGTGGC
GGCAACTGGATTTATGAGTGGGCCCCGGATCTTTGTGAAGGAACCTTACTTCTGT
GGTGTGACATAATTGGACAAACTACCTACAGAGATTTAAAGCTCTAAGGTAAATA
TAAAATTTTTAAGTGTATAATGTGTTAAACTACTGATTCTAATTGTTTGTGTATT
TTAGATTCCAACCTATGGAACTGATGAATGGGAGCAGTGGTGGAATGCCTTTAAT
GAGGAAAACCTGTTTTGCTCAGAAGAAATGCCATCTAGTGATGATGAGGCTACTG
CTGACTCTCAACATTCTACTCCTCCAAAAAAGAAGAGAAAGGTAGAAGACCCCAA
GGACTTTCCTTCAGAATTGCTAAGTTTTTTGAGTCATGCTGTGTTTAGTAATAGA
ACTCTTGCTTGCTTTGCTATTTACACCACAAAGGAAAAAGCTGCACTGCTATACA
AGAAAATTATGGAAAAATATTCTGTAACCTTTATAAGTAGGCATAACAGTTATAA
TCATAACATACTGTTTTTTCTTACTCCACACAGGCATAGAGTGTCTGCTATTAAT
AACTATGCTCAAAAATTGTGTACCTTTAGCTTTTTAATTTGTAAAGGGGTTAATA
AGGAATATTTGATGTATAGTGCCTTGACTAGAGATCATAATCAGCCATACCACAT
TTGTAGAGGTTTTACTTGCTTTAAAAAACCTCCCACACCTCCCCCTGAACCTGAA
ACATAAAATGAATGCAATTGTTGTTGTTAACTTGTTTATTGCAGCTTATAATGGT
TACAAATAAAGCAATAGCATCACAAATTTCACAAATAAAGCATTTTTTTCACTGC
ATTCTAGTTGTGGTTTGTCCAAACTCATCAATGTATCTTATCATGTCTGGATCAA
CTGGATAACTCAAGCTAACCAAAATCATCCCAAACTTCCCACCCCATACCCTATT
ACCACTGCCAATTACCTAGTGGTTTCATTTACTCTAAACCTGTGATTCCTCTGAA
TTATTTTCATTTTAAAGAAATTGTATTTGTTAAATATGTACTACAAACTTAGTAG
TTGGAAGGGCTAATTCACTCCCAAAGAAGACAAGATATCCTTGATCTGTGGATCT
ACCACACACAAGGCTACTTCCCTGATTAGCAGAACTACACACCAGGGCCAGGGGT
CAGATATCCACTGACCTTTGGATGGTGCTACAAGCTAGTACCAGTTGAGCCAGAT
AAGGTAGAAGAGGCCAATAAAGGAGAGAACACCAGCTTGTTACACCCTGTGAGCC
TGCATGGGATGGATGACCCGGAGAGAGAAGTGTTAGAGTGGAGGTTTGACAGCCG
CCTAGCATTTCATCACGTGGCCCGAGAGCTGCATCCGGAGTACTTCAAGAACTGC
TGATATCGAGCTTGCTACAAGGGACTTTCCGCTGGGGACTTTCCAGGGAGGCGTG
GCCTGGGCGGGACTGGGGAGTGGCGAGCCCTCAGATCCTGCATATAAGCAGCTGC
TTTTTGCCTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGAC
CCTTTTAGTCAGTGTGGAAAATCTCTAGCAGTGGCGCCCGAACAGGGACTTGAAA
GCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAG
CGGAGGCTAGAAGGAGAGAGATGGGTGCGAGAGCGTCAGTATTAAGCGGGGGAGA
ATTAGATCGCGATGGGAAAAAATTCGGTTAAGGCCAGGGGGAAAGAAAAAATATA
AATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCA
TCCCTTCAGACAGGATCAGAAGAACTTAGATCATTATATAATACAGTAGCAACCC
TCTATTGTGTGCATCAAAGGATAGAGATAAAAGACACCAAGGAAGCTTTAGACAA
GATAGAGGAAGAGCAAAACAAAAGTAAGACCACCGCACAGCAAGCGGCCGGTGAT
CTTCAGACCTGGACGATATATATGAGGGACAATTGGAGAAGTGAATTATATAAAT
ATAAAGTAGTAAAAATTGAACCATTAGGAGTAGCACCCACCAAGGCAAAGAGAAG
AGTGGTGCAGAGAGAAAAAAGAGCAGTGGGAATAGGAGCTTTGTTCCTTGGGTTC
TTGGGAGCAGCAGGAAGCACTATGGGCGCAGCGTCAATGACGCTGACGGTACAGG
CCAGACAATTATTGTCTGGTATAGTGCAGCAGCAGAACAATTTGCTGAGGGCTAT
TGAGGCGCAACAGCATCTGTTGCAACTCACAGTCTGGGGCATCAAGCAGCTCCAG
GCAAGAATCCTGGCTGTGGAAAGATACCTAAAGGATCAACAGCTCCTGGGGATTT
GGGGTTGCTCTGGAAAACTCATTTGCACCACTGCTGTGCCTTGGAATGCTAGTTG
GAGTAATAAATCTCTGGAACAGATTTGGAATCACACGACCTGGATGGAGTGGGAC
AGAGAAATTAACAATTACACAAGCTTAATACACTCCTTAATTGAAGAATCGCAAA
ACCAGCAAGAAAAGAATGAACAAGAATTATTGGAATTAGATAAATGGGCAAGTTT
GTGGAATTGGTTTAACATAACAAATTGGCTGTGGTATATAAAATTATTCATAATG
ATAGTAGGAGGCTTGGTAGGTTTAAGAATAGTTTTTGCTGTACTTTCTATAGTGA
ATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGA
GACAGATCCATTCGATTAGTGAACGGATCTCGACGGTCGCCAAATGGCAGTATTC
ATCCACAATTTTAAAAGAAAAGGGGGGATTGGGGGGTACAGTGCAGGGGAAAGAA
TAGTAGACATAATAGCAACAGACATACAAACTAAAGAATTACAAAAACAAATTAC
AAAAATTCAAAATTTTCGGGTTTATTACAGGGACAGCAGAGATCCAGTTTGGATC
GATAAGCTTGATATCGAATTCGGAGCACTGTCCTCCGAACGTCGGAGCACTGTCC
TCCGAACGTCGGAGCACTGTCCTCCGAACGTCGGAGCACTGTCCTCCGAACGGAG
CATGTCCTCCGAACGTCGGAGCACTGTCCTCCGAACGACTAGTTAGGCGTGTACG
GTGGGAGGCCTATATAAGCAGAGCTCGTTTAGTGAACCGTCAGATCGCCTGGAGA
CGCCATCCACGCTGTTTTGACCTCCATAGAAGACACCGGGACCGATCCAGCCTCT
CGACATTCGTTGGATCCGCCACCATGAAATACACTTCCTATATACTCGCTTTTCA
ACTGTGCATCGTGCTTGGTAGCTTGGGCTGCTATTGTCAGGACCCCTATGTGAAA
GAAGCTGAGAACCTTAAGAAGTATTTTAATGCTGGTCACTCTGACGTGGCGGACA
ATGGGACATTGTTCCTGGGTATTTTGAAGAATTGGAAGGAAGAATCAGATAGAAA
AATAATGCAGTCACAGATCGTGTCCTTCTACTTCAAACTTTTCAAAAATTTCAAG
GACGACCAGTCCATTCAGAAGTCAGTTGAAACAATCAAGGAAGACATGAACGTGA
AATTTTTCAATAGCAATAAAAAGAAAAGGGATGATTTTGAGAAGTTGACAAATTA
CTCCGTGACTGACCTCAACGTCCAAAGAAAAGCTATACACGAGTTGATCCAAGTT
ATGGCCGAGTTGAGTCCGGCGGCGAAAACAGGAAAACGAAAGAGATCCCAAATGC
TGTTTAGAGGCCGCCGCGCAAGTCAGTAGGGATCCTTGACTTGCGGCCCCCCTCT
CCCTCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGC
CCGGAAACCTGGCCCTGTCTTCTTGACGAGCATTCCTAGGGGTCTTTCCCCTCTC
GCCAAAGGAATGCAAGGTCTGTTGAATGTCGTGAAGGAAGCAGTTCCTCTGGAAG
CTTCTTGAAGACAAACAACGTCTGTAGCGACCCTTTGCAGGCAGCGGAACCCCCC
ACCTGGCGACAGGTGCCTCTGCGGCCAAAAGCCACGTGTATAAGATACACCTGCA
AAGGCGGCACAACCCCAGTGCCACGTTGTGAGTTGGATAGTTGTGGAAAGAGTCA
AATGGCTCTCCTCAAGCGTATTCAACAAGGGGCTGAAGGATGCCCAGAAGGTACC
CCATTGTATGGGATCTGATCTGGGGCCTCGGTGCACATGCTTTACATGTGTTTAG
TCGAGGTTAAAAAAACGTCTAGGCCCCCCGAACCACGGGGACGTGGTTTTCCTTT
GAAAAACACGATGATAAATGAGCGCTGGCGGGTCCATGGTGAGCAAGGGCGAGGA
GGATAACATGGCCATCATCAAGGAGTTCATGCGCTTCAAGGTGCACATGGAGGGC
TCCGTGAACGGCCACGAGTTCGAGATCGAGGGCGAGGGCGAGGGCCGCCCCTACG
AGGGCACCCAGACCGCCAAGCTGAAGGTGACCAAGGGTGGCCCCCTGCCCTTCGC
CTGGGACATCCTGTCCCCTCAGTTCATGTACGGCTCCAAGGCCTACGTGAAGCAC
CCCGCCGACATCCCCGACTACTTGAAGCTGTCCTTCCCCGAGGGCTTCAAGTGGG
AGCGCGTGATGAACTTCGAGGACGGCGGCGTGGTGACCGTGACCCAGGACTCCTC
CCTGCAGGACGGCGAGTTCATCTACAAGGTGAAGCTGCGCGGCACCAACTTCCCC
TCCGACGGCCCCGTAATGCAGAAGAAGACCATGGGCTGGGAGGCCTCCTCCGAGC
GGATGTACCCCGAGGACGGCGCCCTGAAGGGCGAGATCAAGCAGAGGCTGAAGCT
GAAGGACGGCGGCCACTACGACGCTGAGGTCAAGACCACCTACAAGGCCAAGAAG
CCCGTGCAGCTGCCCGGCGCCTACAACGTCAACATCAAGTTGGACATCACCTCCC
ACAACGAGGACTACACCATCGTGGAACAGTACGAACGCGCCGAGGGCCGCCACTC
CACCGGCGGCATGGACGAGCTGTACAAGTAGGGCCGCAACTCCCACCTGCAACAT
GCGTGACTGACTGAGGCCGCGACTCTAGAGTCGACCTGCACGAGGTTAACGAATT
CTACCGGGTAGGGGAGGCGCTTTTCCCAAGGCAGTCTGGAGCATGCGCTTTAGCA
GCCCCGCTGGGCACTTGGCGCTACACAAGTGGCCTCTGGCCTCGCACACATTCCA
CATCCACCGGTAGGCGCCAACCGGCTCCGTTCTTTGGTGGCCCCTTCGCGCCACC
TTCTACTCCTCCCCTAGTCAGGAAGTTCCCCCCCGCCCCGCAGCTCGCGTCGTGC
AGGACGTGACAAATGGAAGTAGCACGTCTCACTAGTCTCGTGCAGATGGACAGCA
CCGCTGAGCAATGGAAGCGGGTAGGCCTTTGGGGCAGCGGCCAATAGCAGCTTTG
CTCCTTCGCTTTCTGGGCTCAGAGGCTGGGAAGGGGTGGGTCCGGGGGCGGGCTC
AGGGGGGGGCTCAGGGGGGGGGCGGGCGCCCGAAGGTCCTCCGGAGGCCCGGCAT
TCTGCACGCTTCAAAAGCGCACGTCTGCCGCGCTGTTCTCCTCTTCCTCATCTCC
GGGCCTTTCGACCTGCAGCCCAAGCTTACCATGAGCGAGCTGATTAAGGAGAACA
TGCACATGAAGCTGTACATGGAGGGCACCGTGGACAACCATCACTTCAAGTGCAC
ATCCGAGGGCGAAGGCAAGCCCTACGAGGGCACCCAGACCATGAGAATCAAGGTG
GTCGAGGGCGGCCCTCTCCCCTTCGCCTTCGACATCCTGGCTACTAGCTTCCTCT
ACGGCAGCAAGACCTTCATCAACCACACCCAGGGCATCCCCGACTTCTTCAAGCA
GTCCTTCCCTGAGGGCTTCACATGGGAGAGAGTCACCACATACGAAGACGGGGGC
GTGCTGACCGCTACCCAGGACACCAGCCTCCAGGACGGCTGCCTCATCTACAACG
TCAAGATCAGAGGGGTGAACTTCACATCCAACGGCCCTGTGATGCAGAAGAAAAC
ACTCGGCTGGGAGGCCTTCACCGAGACGCTGTACCCCGCTGACGGCGGCCTGGAA
GGCAGAAACGACATGGCCCTGAAGCTCGTGGGCGGGAGCCATCTGATCGCAAACA
TCAAGACCACATATAGATCCAAGAAACCCGCTAAGAACCTCAAGATGCCTGGCGT
CTACTATGTGGACTACAGACTGGAAAGAATCAAGGAGGCCAACAACGAGACCTAC
GTCGAGCAGCACGAGGTGGCAGTGGCCAGATACTGCGACCTCCCTAGCAAACTGG
GGCACAAGCTTAATTAATGCAGGCATGCAAGCTTGATATCAAGCTTATCGATAAT
CAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGC
TTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTG
CTGACGCAACCCCCACTGGTTGGGGCATTGCCACCACCTGTCAGCTCCTTTCCGG
GACTTTCGCTTTCCCCCTCCCTATTGCCACGGCGGAACTCATCGCCGCCTGCCTT
GCCCGCTGCTGGACAGGGGCTCGGCTGTTGGGCACTGACAATTCCGTGGTGTTGT
CGGGGAAATCATCGTCCTTTCCTTGGCTGCTCGCCTGTGTTGCCACCTGGATTCT
GCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTC
AGACGAGTCGGATCTCCCTTTGGGCCGCCTCCCCGCAT

The single underlined sequence is the Gal4 UAS sequence. The double underlined sequence is the IFNG sequence. The italics sequence is the IRES sequence. The bold sequence is the mCherry sequence.

### C. Vectors

Disclosed are vectors comprising any of the nucleic acid constructs disclosed herein.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be a retroviral vector. In some aspects, the vector can be a non-viral vector, such as a DNA based vector.

In some aspects, the vector can be pHR, pRRLSIN, or SFG.

### 1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding encoding a VMD2 promoter operably linked to a nucleic acid sequence encoding Rap1a.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### D. Proteins

Disclosed are proteins encoded by the nucleic acid constructs described herein. Disclosed are proteins comprising a scFv; a notch transmembrane domain; and a transcription factor. Disclosed are proteins comprising a scFv; a notch transmembrane domain; and a transcription activator.

In some aspects, the scFv can be any of the scFvs disclosed herein. In some aspects, the notch transmembrane domain can be any of the notch transmembrane domains disclosed herein. In some aspects, the transcription activator can be any of the transcription activators disclosed herein.

### E. Variants

Disclosed herein are variants of the disclosed nucleic acid constructs, vectors or proteins.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologous to a reference sequence. In some aspects, variants include only those variants that retain the same activity as the wild type or reference sequence. In some aspects, a reference sequence can be a scFv, notch transmembrane domain or transcription activator nucleic acid sequence or a scFv, notch transmembrane domain or transcription activator protein sequence. A “variant” can mean a difference in some way from the reference sequence other than just a simple deletion of an N- and/or C-terminal nucleotide. Variants can also or alternatively include at least one substitution and/or at least one addition. There may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence.

Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence a scFv, notch transmembrane domain or transcription activator nucleci acid sequence or protein sequence).

### F. Compositions

Disclosed are compositions comprising the disclosed nucleic acid constructs, vectors, proteins or recombinant cells. Disclosed are compositions comprising nucleic acid constructs comprising a promoter; a nucleic acid sequence encoding a scFv; a nucleic acid sequence encoding a notch transmembrane domain; and a nucleic acid sequence encoding a transcription factor.

Also disclosed are compositions comprising a vector, such as a viral vector, comprising a nucleic acid construct comprising a promoter; a nucleic acid sequence encoding a scFv; a nucleic acid sequence encoding a notch transmembrane domain; and a nucleic acid sequence encoding a transcription factor.

In some aspects, the composition can be a pharmaceutical composition (e.g., formulation, preparation, medicament) comprising, or consisting essentially of, or consisting of as an active ingredient, a nucleic acid construct, vector, protein or recombinant cell as described herein, and a pharmaceutically acceptable carrier, diluent, or excipient.

Disclosed are composition and formulations of the disclosed nucleic acid constructs, vectors, proteins or recombinant cells with a pharmaceutically acceptable carrier or diluent

### 1. Delivery of Compositions

In the methods described herein, delivery (or administration) of the compositions to cells can be via a variety of mechanisms. As defined above, disclosed herein are compositions comprising any one or more of the peptides, nucleic acids, and/or vectors described herein can be used to produce a composition which can also include a carrier such as a pharmaceutically acceptable carrier. For example, disclosed are pharmaceutical compositions, comprising the peptides disclosed herein, and a pharmaceutically acceptable carrier.

While it may possible for the disclosed nucleic acid constructs, vectors, proteins or recombinant cells to be used (e.g., administered) alone, it is often preferable to present it as a composition or formulation e.g. with a pharmaceutically acceptable carrier or diluent.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC: Cholesterol: peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed nucleic acid constructs, vectors, and proteins.

### G. Recombinant Cells

Disclosed are recombinant cells comprising one or more of the disclosed nucleic acid constructs, vectors, or proteins. For example, disclosed are recombinant cells comprising a nucleic acid construct, wherein the nucleic acid construct comprises a promoter; a nucleic acid sequence encoding a scFv; a nucleic acid sequence encoding a notch transmembrane domain; and a nucleic acid sequence encoding a transcription factor.

In some aspects, the recombinant cell can further comprise a response construct comprising an UAS; a promoter controlled by the UAS; and a gene encoding IFNγ operably linked to the promoter.

In some aspects, the cell is a mammalian cell. In some aspects, the cell is a T cell. In some aspects, the cell is a natural killer (NK) cell. In some aspects, the cell is a TCR deficient T cell.

In some aspects, the cell can be a primary cell or a cell line. In some aspects, the cell can be a J76 T cell or a TCR-deficient J76 T cell. In some aspects, the cell can be a primary cell derived from a patient. In some aspects, the cell can be autologous or allogeneic to a patient receiving the cell.

### H. Methods

Disclosed are methods of increasing human leukocyte antigen class I (HLA-I) on the surface of a tumor cell in a subject comprising administering to the subject one or more of the recombinant cells or compositions comprising a recombinant cell disclosed herein.

Also disclosed are methods of increasing HLA-I on the surface of a tumor cell in a subject comprising administering to the subject one or more of the nucleic acid constructs, vectors, or proteins disclosed herein.

In some aspects, the scFv used in the methods is based on the presence of a tumor specific marker or a marker over-expressed by a tumor cell. In some aspects, the tumor cell expresses one or more of GD2, B7H3, CD171, and GPC2.

In some aspects, the tumor cell can be, but is not limited to, a cell from a neuroblastoma, retinoblastoma, pediatric sarcomas (such as Ewings sarcoma, desmoplastic small round cell tumors, rhabdomyosarcoma and osteosarcoma), brain tumors (such as diffuse midline glioma), as well as adult cancers including small-cell lung cancer, melanoma, soft-tissue sarcomas, colon cancer, and lung cancer.

In some aspects, the transcription activator binds to the UAS in the response construct. The transcription activator can be present in the recombinant cells administered to the subject or present in the nucleic acid constructs, vectors, or proteins administered to the subject. The UAS can be present in the recombinant cells administered to the subject or present in the nucleic acid constructs, vectors, or proteins administered to the subject. In some aspects, the recombinant cells administered to a subject can have both the transcription activator and the UAS.

In some aspects, activation of the UAS activates the promoter operably linked to the of IFNγ of the response construct.

In some aspects, the recombinant cell produces IFNγ only in the presence of antigen-positive tumor cells. Antigen-positive tumor cells are those cells that express an antigen specific to the scFv present on the surface of the recombinant cells administered to the subject. Upon binding of the scFv to the specific antigen on the tumor cell, a transcriptional activation cascade occurs in the cell which leads to production of IFNγ. In some aspects, the recombinant cell secretes IFNγ only in the presence of antigen-positive tumor cells.

In some aspects, the recombinant cell can be administered to the subject via intravenous, intratumoral, intraperitoneal, or intrathecal injection. In some aspects, any of the disclosed or previously known routes of administration can be used.

In some aspects, PD-1 and/or PD-L1 expression on the tumor cells is not altered. In some aspects, PD-1 and/or PD-L1 expression on the tumor cells is not upregulated. In some aspects, simultaneous induction of PD-1 and/or PD-L1 and the resulting inhibition of tumor-specific T cells can counteract the beneficial effect of HLA upregulation. Thus, in some aspects, the induction of HLA, but not PD-1 or PD-L1, by recombinant T cells can have superior anti-tumor activity compared to other approaches, such as injection of recombinant IFNγ, inducing both HLA and PD-1 and/or PD-L1. In some aspects, the lack of PD-1 and/or PD-L1 upregulation by recombinant T cells can be related to the relatively low concentrations of IFNγ produced by the cells or the specific context in which it is secreted, ie. after formation of an immune synapse by a non-activated T cell.

In some aspects, the disclosed methods can be used in combination with a known anti-cancer treatment. For example, the disclosed methods can be used in combination with chemotherapy.

The term “treatment,” as used herein in the context of treating a disease or disorder, can relate generally to treatment and therapy of a human subject or patient, in which some desired therapeutic effect is achieved, for example, the inhibition of the progress of the disease or disorder, and can include a reduction in the rate of progress, a halt in the rate of progress, regression of the disease or disorder, amelioration of the disease or disorder, and cure of the disease or disorder. Treatment as a prophylactic measure (i.e., prophylaxis, prevention) is also included.

In some aspects, the disclosed compositions comprising the disclosed nucleic acid constructs, vectors, proteins or recombinant cells can be delivered in a therapeutically-effective amount. In some aspects, the disclosed compositions comprising the disclosed nucleic acid constructs, vectors, proteins or recombinant cells can be delivered in a therapeutically-effective amount.

The term “therapeutically-effective amount” as used herein, refers to the amount of the disclosed compositions comprising the disclosed nucleic acid constructs, vectors, proteins or recombinant cells that is effective for producing some desired therapeutic effect, commensurate with a reasonable benefit/risk ratio, when administered in accordance with a desired treatment regimen.

Similarly, the term “prophylactically effective amount,” as used herein refers to the amount of the disclosed compositions comprising the disclosed nucleic acid constructs, vectors, proteins or recombinant cells that is effective for producing some desired prophylactic effect, commensurate with a reasonable benefit/risk ratio, when administered in accordance with a desired treatment regimen. “Prophylaxis” as used herein refers to a measure which is administered in advance of detection of a symptomatic condition, disease or disorder with the aim of preserving health by helping to delay, mitigate or avoid that particular condition, disease or disorder.

In some aspects, the disclosed methods or compositions can be combined with other therapies, whether symptomatic or disease modifying.

The term “treatment” includes combination treatments and therapies, in which two or more treatments or therapies are combined, for example, sequentially or simultaneously. For example it may be beneficial to combine treatment with a compositions comprising the disclosed nucleic acid constructs, vectors, proteins or recombinant cells as described herein with one or more other (e.g., 1, 2, 3, 4) agents or therapies. Appropriate examples of co-therapeutics are known to those skilled in the art based one the disclosure herein. Typically the co-therapeutic can be any known in the art which it is believed may give therapeutic effect in treating the diseases or disorders described herein, subject to the diagnosis of the individual being treated.

The particular combination would be at the discretion of the physician who would also select dosages using his/her common general knowledge and dosing regimens known to a skilled practitioner.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits comprising one or more of the disclosed nucleic acid constructs, vectors, proteins or recombinant cells or one or more disclosed compositions comprising the disclosed nucleic acid constructs, vectors, proteins or recombinant cells.

The disclosed kits can also include a vector backbone. In some aspects, the kit can also include cells and/or culture media.

### EXAMPLES

### A. Example 1

### 1. Results

As a model disease to demonstrate the efficacy of the current approach, neuroblastoma was chosen. Neuroblastoma shows frequent loss of HLA and in vitro studies confirmed upregulation of HLA on these cells after incubation with IFNG. Neuroblastoma is the most common solid extracranial childhood tumor. It accounts for 8-10% of all pediatric cancers and 12-15% of cancer-related deaths in children. In particular, the survival of pediatric patients with high-risk neuroblastoma remains close to only 50%. The addition of immunotherapy utilizing an anti-GD2 antibody plus cytokines improved event-free and overall survival. However, although outcomes for patients with high-risk neuroblastoma have markedly improved over the past two decades, current therapies for these patients remain suboptimal. Recently, the efficacy of TCR-transduced T cells targeting NY-ESO-1 has been described in neuroblastoma in a mouse xenograft model showing high spontaneous expression of HLA. Treatment resulted in a significant delay of tumor progression in mice and enhanced survival in recipient animals. However, HLA downregulation is frequent in neuroblastoma, indicating that the majority of patients would be unlikely to respond to treatments like this.

i. Induction of HLA Expression By T Cells Engineered to Secrete IFNG Constitutively

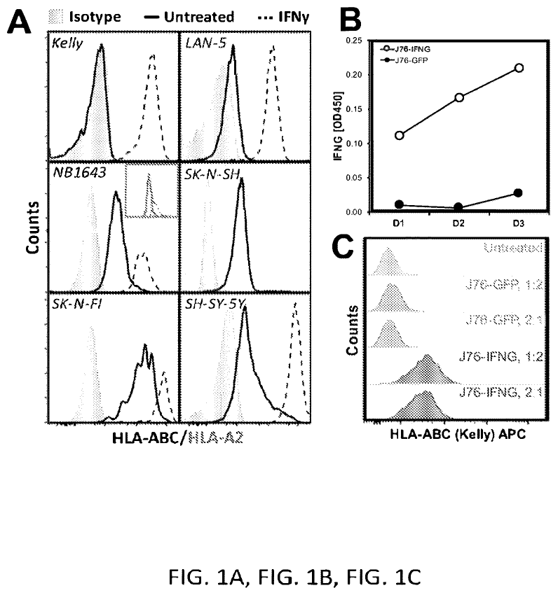

Treatment with IFNG can enhance the functional avidity of TCR-transduced T cells and can improve antitumor responses in clinical studies. The current data show that IFNG, when provided as a recombinant protein, induced the upregulation of HLA class I (ABC) on all tested neuroblastoma cell lines. Importantly, IFNG also upregulated HLA-A2 on neuroblastoma cell line NB1643. (FIG. 1A). We next set out to determine whether T cells expressing IFNG from an expression construct are able to secrete sufficient levels of the cytokine to induce HLA class I expression in Neuroblastoma cells. Using lentiviral gene transfer, we generated J76 T cells expressing IFNG or GFP constitutively. Cells transduced with the IFNγ construct secreted increasing IFNG over the course of 3 days (FIG. 1B). Importantly, we also observed strong upregulation of HLA class I by neuroblastoma cells in the presence of IFNG expressing cells but not GFP expressing cells (FIG. 1C).

ii. Development of T Cells Conditionally Expressing IFNγ

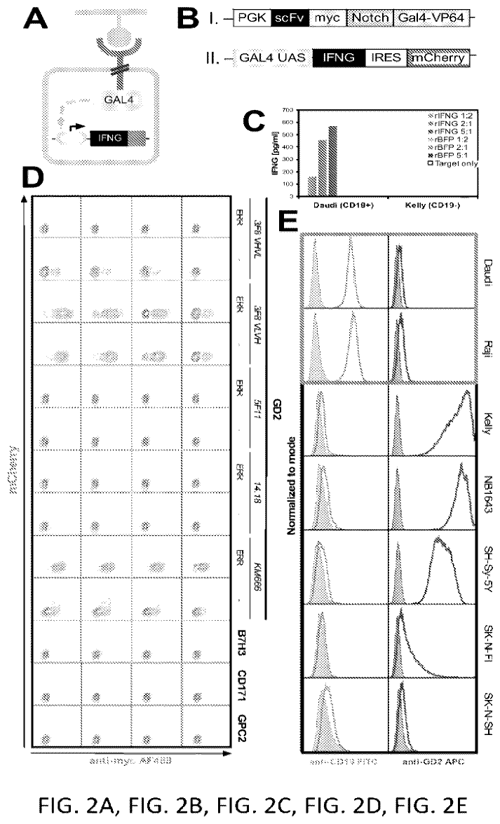

Next, a cell therapy approach was developed based on the previously described synNotch system to conditionally secrete IFNG when encountering tumor cells (FIG. 2A). TCR-deficient J76 T cells expressing a synthetic receptor were generated by combining a CD19 scFv with the Notch 1 regulatory and transmembrane domains, the GAL4 DNA-binding element, and the VP64 transcriptional activator (snCD19, FIG. 2B construct I). In addition, these cells were transduced either with a response element vector containing a PGK promotor that drives constitutive expression of BFP and conditional expression of IFNγ and fluorescent reporter mCherry (rIFNG, FIG. 2B construct II), or with a response construct leading to constitutive expression of mCherry and conditional expression of BFP upon ligand binding (FIG. 2B construct III). Both conditional response elements are under the control of a minimal CMV promoter containing GAL4 upstream activation sequences (UAS), and DNA sequences allowing binding of GAL4. Using these constructs, the ability of the snCD19 cells to secrete IFNG during a co-culture assay with CD19+ lymphoma cell line Daudi but not when co-cultured with the CD19− neuroblastoma cell line Kelly was shown (FIG. 2C). These findings demonstrate that high IFNG levels can be achieved through adaptation of the previously described synNotch receptor system. Next, synNotch receptors specific for neuroblastoma cells were generated using previously described antibodies targeting the antigens GD2, B7H3, CD171, and GPC2 (Table 1-8). J76 cells were then transduced with these receptors and construct II to determine receptor surface expression levels as well as baseline activation, evidenced by the expression of mCherry. Only 3/8 antibody constructs, clones 3F8 VHVL, 3F8 VLVH, and KM666 showed surface expression on J76 cells. The synNotch receptor using KM666 showed substantial expression of mCherry in the absence of GD2-positive target cells indicating high basal signaling. This issue was resolved by extending the core Notch regulatory region to include additional EGF repeats (Table 1-8). However, as the 3F8 constructs showed substantially higher surface expression in the absence of baseline signaling, clone 3F8-VLVH was selected for all subsequent experiments (snGD2) (Table 9). As only GD2-specific receptors showed measurable surface expression, the expression of GD2 was determined on a set of neuroblastoma cell lines using flow cytometry. We found that 4/5 neuroblastoma cell lines showed expression of GD2 (FIG. 2E). Importantly, control lymphoma cell lines Daudi and Raji showed expression of CD19 but not GD2.

TABLE 1
Amino acid sequences of 5F11 heavy and light
chains annotated with linker, with and
without ERR.

5F11 V_{H}-linker-V_{L}
QVKLQQSGPELVEPGASVKISCKTSGYKFTEYTMHWVKQSHGKSLEWIG
GINPNNGGTNYKQKFKGKATLTVDKSSSTAYMELRSLTSEDSAVYYCAR
DTTVPFAYWVQGTTVTVSSGGGGSGGGGSGGGGSDIELTQSPAIMSASP
GEKVTMTCSGSSSISYMHWYQQKPVTSPKRWIYDTSKLASGVPARFSGS
GSGTSYSLTISSMEAVDAATYYCHQRSSYPLTFGAGTQLEIKR
5F1 V_{H}-linker-V_{L}_ERR
QVKLQQSGPELVEPGASVKISCKTSGYKFTEYTMHWVKQSHGKSLEWIG
GINPNNGGTNYKQKFKGKATLTVDKSSSTAYMELRSLTSEDSAVYYCAR
DTTVPFAYWVQGTTVTVSSGGGGSGGGGSGGGGSDIELTQSPAIMSASP
GEKVTMTCSGSSSISYMHWYQQKPVTSPKRWIYDTSKLASGVPARFSGS
GSGTSYSLTISSMEAVDAATYYCHQRSSYPLTFGAGTQLEIKRPCVGSN
PCYNQGTCEPTSENPFYRCLCPAKFNGLLCH

TABLE 2
Amino acid sequences of 14_18 heavy and light
chains annotated with linker, with and
without ERR.

14_18 V_{H}-linker-V_{L}
EVQLVQSGAEVEKPGASVKISCKASGSSFTGYNMNWVRQNIGKSLEWIG
AIDPYYGGTSYNQKFKGRATLTVDKSTSTAYMHLKSLRSEDTAVYYCVS
GMEYWGQGTSVTVSSGGGGSGGGGSGGGGSGGGGSDVVMTQTPLSLPVT
PGEPASISCRSSQSLVHRNGNTYLHWYLQKPGQSPKLLIHKVSNRFSGV
PDRFSGSGSGTDFTLKISRVEAEDLGVYFCSQSTHVPPLTFGAGTKLEL
KR
14_18 V_{H}-linker-V_{L} ERR
EVQLVQSGAEVEKPGASVKISCKASGSSFTGYNMNWVRQNIGKSLEWIG
AIDPYYGGTSYNQKFKGRATLTVDKSTSTAYMHLKSLRSEDTAVYYCVS
GMEYWGQGTSVTVSSGGGGSGGGGSGGGGSGGGGSDVVMTQTPLSLPVT
PGEPASISCRSSQSLVHRNGNTYLHWYLQKPGQSPKLLIHKVSNRFSGV
PDRFSGSGSGTDFTLKISRVEAEDLGVYFCSQSTHVPPLTFGAGTKLEL
KRPCVGSNPCYNQGTCEPTSENPFYRCLCPAKFNGLLCH

TABLE 3
Amino acid sequences of 3F8 heavy and
light chains annotated with linker,
with and without ERR.

3F8 V_{H}-linker-V_{L}
QVQLVESGPGVVQPGRSLRISCAVSGFSVTNYGVHWVRQPPGKGLEW
LGVIWAGGITNYNSAFMSRLTISKDNSKNTVYLQMNSLRAEDTAMYY
CASRGGHYGYALDYWGQGTLVTVSSGGGGSGGGGSGGGGSGGGGSEI
VMTQTPATLSVSAGERVTITCKASQSVSNDVTWYQQKPGQAPRLLIY
SASNRYSGVPARFSGSGYGTEFTFTISSVQSEDFAVYFCQQDYSSFG
QGTKLEIKR
3F8 V_{H}-linker-V_{L}_ERR
QVQLVESGPGVVQPGRSLRISCAVSGFSVTNYGVHWVRQPPGKGLEW
LGVIWAGGITNYNSAFMSRLTISKDNSKNTVYLQMNSLRAEDTAMYY
CASRGGHYGYALDYWGQGTLVTVSSGGGGSGGGGSGGGGSGGGGSEI
VMTQTPATLSVSAGERVTITCKASQSVSNDVTWYQQKPGQAPRLLIY
SASNRYSGVPARFSGSGYGTEFTFTISSVQSEDFAVYFCQQDYSSFG
QGTKLEIKRPCVGSNPCYNQGTCEPTSENPFYRCLCPAKENGLLCH

TABLE 4
Amino acid sequences of 3F8 light and heavy
chains annotated with linker,
with and without ERR.

3F8 V_{L}-linker-V_{H}
EIVMTQTPATLSVSAGERVTITCKASQSVSNDVTWYQQKPGQAPR
LLIYSASNRYSGVPARFSGSGYGTEFTFTISSVQSEDFAVYFCQQ
DYSSFGQGTKLEIKRGGGGSGGGGSGGGGSGGGGSQVQLVESGPG
VVQPGRSLRISCAVSGFSVTNYGVHWVRQPPGKGLEWLGVIWAGG
ITNYNSAFMSRLTISKDNSKNTVYLQMNSLRAEDTAMYYCASRGG
HYGYALDYWGQGTLVTVSS
3F8 V_{L}-linker-V_{H}_ERR
EIVMTQTPATLSVSAGERVTITCKASQSVSNDVTWYQQKPGQAPR
LLIYSASNRYSGVPARFSGSGYGTEFTFTISSVQSEDFAVYFCQQ
DYSSFGQGTKLEIKRGGGGSGGGGSGGGGSGGGGSQVQLVESGPG
VVQPGRSLRISCAVSGFSVTNYGVHWVRQPPGKGLEWLGVIWAGG
ITNYNSAFMSRLTISKDNSKNTVYLQMNSLRAEDTAMYYCASRGG
HYGYALDYWGQGTLVTVSSPCVGSNPCYNQGTCEPTSENPFYRCL
CPAKFNGLLCH

TABLE 5
Amino acid sequences of KM666 heavy and light
chains annotated with linker, with and
without ERR.

KM666 V_{H}-linker-V_{L}
QVQLQESGPGLVKPSQTLSITCTVSGFSLASYNIHWVRQPPGKGL
EWLGVIWAGGSTNYNSALMSRLTISKDNSKNQVFLKMSSLTAADT
AVYYCAKRSDDYSWFAYWGQGTLVTVSSGGGGSGGGGSGGGGSEN
QMTQSPSSLSASVGDRVTMTCRASSSVSSSYLHWYQQKSGKAPKV
WIYSTSNLASGVPSRFSGSGSGTDYTLTISSLQPEDFATYYCQQY
SGYPITFGQGTKVEIKR
KM666 V_{H}-linker-V_{L}_ERR
QVQLQESGPGLVKPSQTLSITCTVSGFSLASYNIHWVRQPPGKGL
EWLGVIWAGGSTNYNSALMSRLTISKDNSKNQVFLKMSSLTAADT
AVYYCAKRSDDYSWFAYWGQGTLVTVSSGGGGSGGGGSGGGGSEN
QMTQSPSSLSASVGDRVTMTCRASSSVSSSYLHWYQQKSGKAPKV
WIYSTSNLASGVPSRFSGSGSGTDYTLTISSLQPEDFATYYCQQY
SGYPITFGQGTKVEIKRPCVGSNPCYNQGTCEPTSENPFYRCLCP
AKFNGLLCH

TABLE 6
Amino acid sequences of GPC2 heavy and
light chains annotated with linker.

GPC2 V_{H}-linker-V_{L}
LISEEDLEVQLVETGGGVVKPGGSLRLSCAASGFTFSDYYMSWIR
QAPGKGLEWVSYISSSGSTIYYADSVKGRFTISRDNSKNTLYLQM
NSLRAEDTAVYYCARESGYDYVFDYWGQGTLVAVSSGGGGSGGGG
SGGGGSDIQMTQSPSTLSAFVGDRVTITCRASQSISSWLAWYQQK
PGKAPKLLIYAASTLQSGVPSRFSGSGSGTEFTLTISSLQPEDFA
TYYCQQLNSYPITFGQGTRLEIKRILDYSF

TABLE 7
Amino acid sequences of B7-H3 heavy and light
chains annotated with linker.

B7-H3 V_{H}-linker-V_{L}
LISEEDLEVQLVESGGGLVQPGGSLRLSCAASGFTFSSFGMHWVR
QAPGKGLEWVAYISSDSSAIYYADTVKGRFTISRDNAKNSLYLQM
NSLRDEDTAVYYCGRGRENIYYGSRLDYWGQGTTVTVSSGGGGSG
GGGSGGGGSDIQLTQSPSFLSASVGDRVTITCKASQNVDTNVAWY
QQKPGKAPKALIYSASYRYSGVPSRFSGSGSGTDFTLTISSLQPE
DFATYYCQQYNNYPFTFGQGTKLEIKILDYSF

TABLE 8
Amino acid sequences of CD171 heavy and light
chains annotated with linker.
CD171 V_{H}-linker-V_{L}
LISEEDLQVQLQQPGAELVKPGASVKLSCKASGYTFTGYWMHWVK
QRPGHGLEWIGEINPSNGRTNYNERFKSKATLTVDKSSTTAFMQL
SGLTSEDSAVYFCARDYYGTSYNFDYWGQGTTLTVSSGGGGSGGG
GSGGGGSDIQMTQSSSSFSVSLGDRVTITCKANEDINNRLAWYQQ
TPGNSPRLLISGATNLVTGVPSRFSGSGSGKDYTLTITSLQAEDF
ATYYCQQYWSTPFTFGSGTELEIKVEILDYSF

TABLE 9
Complete sequence of 3F8 VL_VH_IFNG SN T cells,
annotated with linker and ERR

3F8 V_{H}-linker-V_{L}_ERR
EIVMTQTPATLSVSAGERVTITCKASQSVSNDVTWYQQKPGQAPR
LLIYSASNRYSGVPARFSGSGYGTEFTFTISSVQSEDFAVYFCQQ
DYSSFGQGTKLEIKRGGGGSGGGGSGGGGSGGGGSQVQLVESGPG
VVQPGRSLRISCAVSGFSVTNYGVHWVRQPPGKGLEWLGVIWAGG
ITNYNSAFMSRLTISKDNSKNTVYLQMNSLRAEDTAMYYCASRGG
HYGYALDYWGQGTLVTVSSPCVGSNPCYNQGTCEPTSENPFYRCL
CPAKFNGLLCH
SynNotch
ILDYSFTGGAGRDIPPPQIEEACELPECQVDAGNKVCNLQCNNHA
CGWDGGDCSLNFNDPWKNCTQSLQCWKYFSDGHCDSQCNSAGCLF
DGFDCQLTEGQCNPLYDQYCKDHFSDGHCDQGCNSAECEWDGLDC
AEHVPERLAAGTLVLVVLLPPDQLRNNSFHFLRELSHVLHTNVVF
KRDAQGQQMIFPYYGHEEELRKHPIKRSTVGWATSSLLPGTSGGR
QRRELDPMDIRGSIVYLEIDNRQCVQSSSQCFQSATDVAAFLGAL
ASLGSLNIPYKIEAVKSEPVEPPLPSQLHLMYVAAAAFVLLFFVG
CGVLLSRKRRR
GAL4-VP64
MKLLSSIEQACDICRLKKLKCSKEKPKCAKCLKNNWECRYSPKTK
RSPLTRAHLTEVESRLERLEQLFLLIFPREDLDMILKMDSLQDIK
ALLTGLFVQDNVNKDAVTDRLASVETDMPLTLRQHRISATSSSEE
SSNKGQRQLTVSAAAGGSGGSGGSDALDDFDLDMLGSDALDDFDL
DMLGSDALDDFDLDMLGSDALDDFDLDMLGS
GAL4-UAS
GALSSERRSTVLRTSEHCPPNVGALSSE
IFNG-ORF
MKYTSYILAFQLCIVLGSLGCYCQDPYVKEAENLKKYFNAGHSDV
ADNGTLFLGILKNWKEESDRKIMQSQIVSFYFKLFKNFKDDQSIQ
KSVETIKEDMNVKFFNSNKKKRDDFEKLTNYSVTDLNVQRKAIHE
LIQVMAELSPAAKTGKRKRSQMLFRGRRASQ
Mcherry
MVSKGEEDNMAIIKEFMRFKVHMEGSVNGHEFEIEGEGEGRPYEG
TQTAKLKVTKGGPLPFAWDILSPQFMYGSKAYVKHPADIPDYLKL
SFPEGFKWERVMNFEDGGVVTVTQDSSLQDGEFIYKVKLRGTNFP
SDGPVMQKKTMGWEASSERMYPEDGALKGEIKQRLKLKDGGHYDA
EVKTTYKAKKPVQLPGAYNVNIKLDITSHNEDYTIVEQYERAEGR
HSTGGMDELYK
TagBFP
MSELIKENMHMKLYMEGTVDNHHFKCTSEGEGKPYEGTQTMRIKV
VEGGPLPFAFDILATSFLYGSKTFINHTQGIPDFFKQSFPEGFTW
ERVTTYEDGGVLTATQDTSLQDGCLIYNVKIRGVNFTSNGPVMQK
KTLGWEAFTETLYPADGGLEGRNDMALKLVGGSHLIANIKTTYRS
KKPAKNLKMPGVYYVDYRLERIKEANNETYVEQHEVAVARYCDLP
SKLGHKLN

iii. Upregulation of HLA on Neuroblastoma Cells by SynNotch T Cells

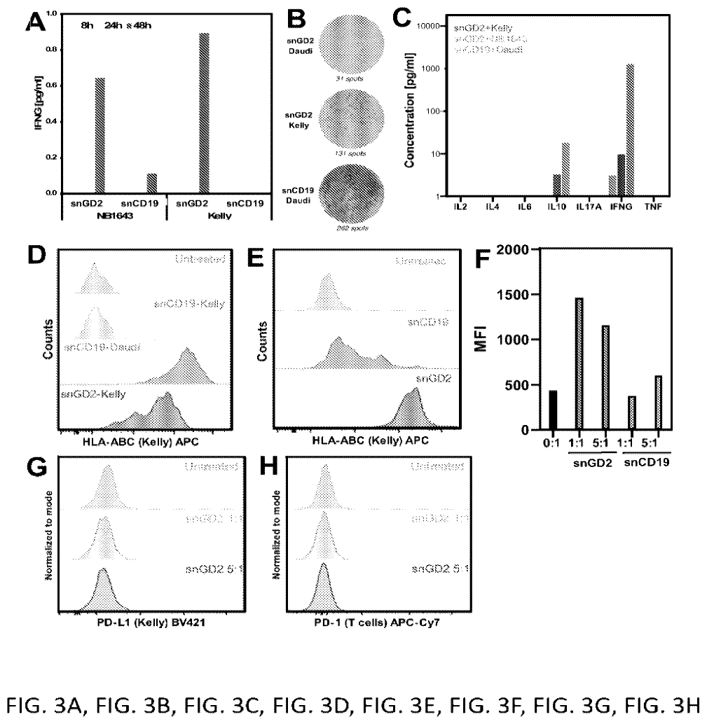

Having demonstrated the expression of IFNG by CD19 synNotch cells in the presence of CD19-positive target cells and the high surface expression of GD2-specific synNotch receptors, next the ability of the snGD2 cells to secrete IFNG only in the presence of GD2-expressing cells was determined. Co-culturing snGD2 or snCD19 cells with GD2-positive neuroblastoma cell lines NB1643 or Kelly, increased levels of IFNG in the presence of cells expressing GD2 by ELISA were observed (FIG. 3A). Expression of IFNG was confirmed in these co-cultures by ELISpot (FIG. 3B). Importantly, IFNG levels from snGD2 cells in the presence of GD2-positive neuroblastoma cells were orders of magnitude lower than those observed in co-cultures containing snCD19 cells and CD19-positive target cells (FIG. 3C). In addition, it was confirmed that synNotch cells specifically secreted IFNG. Unexpectedly, low levels of the cytokine IL-10 was also observed. However, it was unclear whether these relatively low levels of IFNG would be sufficient to induce HLA class I expression on the tumor cells. A set of experiments were performed to determine the ability of snGD2 cells to induce HLA class I on neuroblastoma tumor cells. First, conditioned media was harvested from 48 h co-cultures of synNotch cells with tumor cells and treated neuroblastoma cell line Kelly for 48 h with these supernatants. Strong upregulation of HLA class I on Kelly cells was observed with supernatants obtained from CD19 synNotch cells cultured with CD19-positive Daudi cells (FIG. 3D). In addition, strong upregulation was observed with supernatants from GD2 synNotch cells cultured with GD2-positive Kelly cells, despite very low overall levels of IFNG. Next, snCD19 and snGD2 cells were cultured directly with Kelly cells and HLA class I expression was measured after 48 h. Again, strong induction of HLA class I was observed on Kelly cells by snGD2 cells but only very little induction by snCD19 cells (FIG. 3E). The same observation was made using NB1643 cells (FIG. 3F). Importantly, it has previously been described that treatment with recombinant IFNG can induce the expression of the immune checkpoint PD-1 in T cells as well as its ligand PD-L1 in tumor cells (16-18). Therefore, it was determined whether prolonged incubation with snGD2 cells not only leads to upregulation of HLA class I but also of PD-1/PD-L1. However, it was found that treatment with synNotch T cells for 48 h did not lead to any increase in PD-L1 on the neuroblastoma cells (FIG. 3G) or PD-1 on the synNotch cells themselves (FIG. 3H). Taken together the neuroblastoma-specific synNotch cells only secreted IFNG in the presence of GD2-positive cells, which led to strong upregulation of HLA class I on neuroblastoma cells. Importantly, the levels of IFNG secreted by snGD2 cells were orders of magnitude lower than those produced by snCD19 cells, indicating a low likelihood of measurable systemic levels of IFNG and limited off-target effects, which is also illustrated by the lack of upregulation of PD-1 and PD-L1 in T cells and neuroblastoma cells, respectively.

iv. Cytotoxic Activity of NY-ESO1-and PRAME-Specific TCR-Transduced T Cells

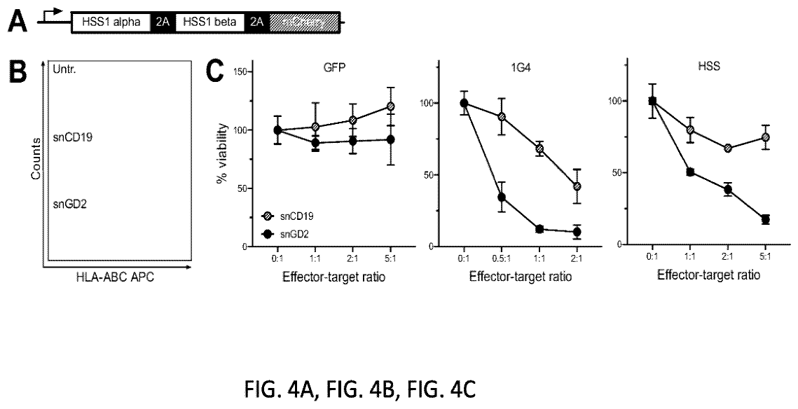

IFNG at low levels is unlikely to have direct cytotoxic effects on tumor cells themselves. The goal of this approach is to instead augment pre-existing anti-tumor T cell responses or adoptively transferred TCR-transgenic T cells by rendering the tumor cells visible to these cells. Therefore, primary human T cells were generated expressing a TCR, clone 1G4 recognizing the widely expressed tumor antigen NY-ESO-1. In addition, a highly potent TCR against PRAME, clone HSS1, has been obtained (FIG. 4A). Both receptors were cloned into the lentiviral backbone (FIG. 4A), high-titered lentivirus was generated, and TCR-transgenic T cells were produced from peripheral blood mononuclear cells from an HLA-A2+ healthy donor.

Treating the HLA-A2+ neuroblastoma cell line SK-N-DZ, which shows very low HLA-ABC mRNA expression, with snGD2 T cells led to a substantial increase in HLA-ABC expression compared to cells treated with snCD19 T cells (FIG. 4B). Importantly, pretreatment with snGD2 T cells resulted in drastically increased killing of SK-N-DZ cells by TCR-transgenic T cells targeting the tumor antigens NY-ESO-1 and PRAME (FIG. 4C).

v. Upregulation of HLA on a Xenograft Neuroblastoma Model Treated with SynNotch T Cells

Having demonstrated the activity of the synNotch T cells in vitro, in vivo findings were next demonstrated.

In murine studies, it was observed that direct intratumoral injection of recombinant IFNG into subcutaneous murine melanoma induced transient CXCL9 and CXCL10 production that returned to baseline levels within 36 hours. A more sustained delivery of IFNG to the tumor microenvironment, rather than one intramural injection of recombinant IFNG, may be required to induce HLA upregulation on tumor cells and stimulate T-cell trafficking to tumors.

NOD.Cg-PrkdcscidIl2rgtm1Wjl/SzJ (NSG) mice engrafted with the human neuroblastoma cell line Kelly were used for the following experiments. A total of 1×10⁶ Kelly cells were injected subcutaneously with Matrigel into both flanks. Once tumors reached a diameter of 5 mm (after 3-4 days), 1×10⁶ tumor GD2-specific IFNG-synNotch T cells were injected directly into the tumor. Three days after treatment, mice were euthanized and their tumors were harvested and analyzed by IHC for HLA induction.

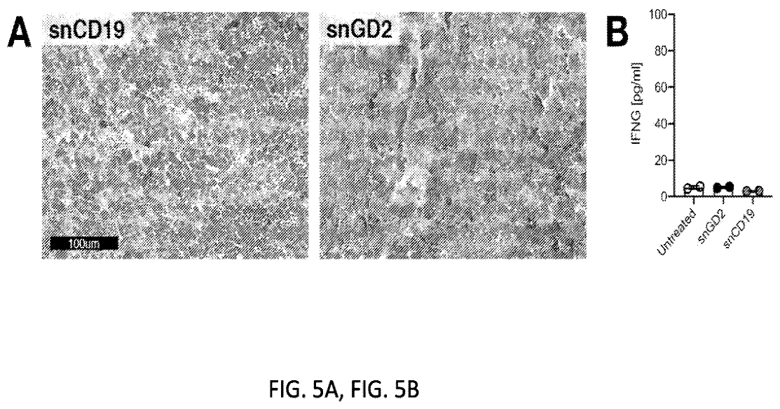

snGD2 T cell-treated mice showed HLA upregulation in tumors as evidenced by immunohistochemistry (IHC) compared to snCD19 T cell-treated mice (FIG. 5A). Importantly, in the peripheral blood of the animals, systemically increased levels of INFG in animals treated with snGD2 T cells was not observed (FIG. 5B) indicating localized secretion of IFNG in the tumor microenvironment, a distinct advantage of snGD2 cells over systemic intravenous injection of recombinant IFNG.

The findings in this study demonstrate the in vivo efficacy of GD2-specific IFNG-synNotch T cells injected intratumorally on HLA upregulation in neuroblastoma tumors.

### 2. Conclusions

Neuroblastoma-specific synNotch cells can be engineered and they specifically produce IFNG only in the presence of antigen-positive tumor cells. The secreted IFNG drastically upregulates HLA class I on these tumor cells in vitro and in vivo, rendering them more susceptible to killing by tumor-specific TCR-transgenic T cells. In addition, upregulation of the immune checkpoint PD-1 was not observed on T cells or its ligand PD-L1 on tumor cells after treatment, which can be related to the relatively low level of IFNG secreted by these cells. Finally, systemically increased levels of IFNG were not observed in animals treated with snGD2 cells, indicating that systemic toxicities are unlikely. Taken together, a highly efficient and safe process for the targeted delivery of IFNG, leading to the upregulation of HLA class I, which in turn enhances T cell-mediated killing of tumor cells has been shown.

### B. Example 2

### 1. Scientific/Clinical Background and Disease Relevancy

Neuroblastoma is the most common solid extracranial tumor of childhood. Neuroblastoma accounts for 8-10% of all pediatric cancers and 12-15% of cancer-related deaths in children. In particular, the survival of pediatric patients with high-risk neuroblastoma remains close to only 50%. The addition of immunotherapy utilizing an anti-GD2 antibody plus cytokines improved event-free and overall survival. However, although outcomes for patients with high-risk neuroblastoma have markedly improved over the past two decades, current therapies for these patients remain suboptimal. New therapeutic strategies are needed in particular for those patients who cannot be cured using currently available approaches.

Patient-derived T lymphocytes engineered to express either chimeric antigen receptors or tumor antigen specific T cell receptors (TCR) have recently shown remarkable clinical activity in hematologic and solid malignancies. These strategies rely on the presence of proteins specifically expressed by tumor cells. Cancer-germline antigens (CGA) are attractive targets for the immunotherapy of neuroblastoma. Their expression in neuroblastoma has been shown to be high compared to other solid malignancies: a CGA that has been targeted in different cancers using TCR-transduced T cells, NY-ESO-1, is expressed in 23-82% of patients as determined by immunohistochemistry; another CGA, PRAME, is expressed in 93% of patients as determined by RT-PCR. The efficacy of TCR-transduced T cells targeting NY-ESO-1 has already been shown in neuroblastoma in a mouse xenograft model. Treatment resulted in a significant delay of tumor progression in mice and enhanced survival in recipient animals. However, responses were not durable suggesting that under selective pressure neuroblastoma cells are able to evade immune recognition. An established mechanism of immune evasion by neuroblastoma cells is the downregulation of HLA class I molecules cloaking the malignant cells from recognition by cytotoxic T cells. Data has shown that HLA class I levels can be increased in neuroblastoma cells through exposure to the cytokines interferon gamma (IFNγ) or tumor necrosis factor alpha (TNFα).

This project upregulates HLA class I in neuroblastoma cell lines by pre-treating them with cytokines using three different approaches: 1) using varying concentrations of recombinant IFNγ, 2) using T cells engineered to constitutively secrete IFNγ, and 3) using T cells engineered to conditionally secrete IFNγ when encountering target cells expressing GD2. The goal is to improve the efficacy of adoptive immunotherapy by reverting downregulation of HLA class I on neuroblastoma cells. Using conditionally cytokine secreting T cells can furthermore both increase the efficiency and reduce the systemic toxicity of IFNγ. To this end, the cytolytic activity of TCR-transduced T cells targeting PRAME and NY-ESO-1 expressed by neuroblastoma cells that have been previously treated by IFNγ, either provided as a recombinant protein or released by cytokine secreting cells, will be determined.

Neuroblastoma is a solid tumor that arises from primordial neural crest cells in pediatric patients. High-risk neuroblastoma treatment includes dose-intensive multimodality therapy, however, only 50% of these patients can be cured by this approach. There is an urgent need to develop new classes of therapeutics to treat childhood cancer, and cellular immunotherapy is emerging as a promising strategy for cancer treatment in children.

Despite progress in the development and refinement of immune based therapies, there are limitations to T cell-mediated approaches. Almost all tumors evade immune responses through multiple mechanisms, including the down regulation of MHC class I, which leads to hyporesponsiveness of antitumor effector cells. Cancer-germline antigens (CGA) are a particularly promising group of targets and their expression in neuroblastoma has been shown to be high compared to other solid malignancies. The efficacy of TCR-transduced T cells targeting the CGA NY-ESO-1 in neuroblastoma has been shown in a mouse xenograft model. Treatment resulted in a significant delay of tumor progression in mice and enhanced the animals' survival. However, responses were not durable suggesting that under selective pressure neuroblastoma cells are able to evade immune-recognition.

An established mechanism of immune evasion by neuroblastoma cells is the downregulation of HLA class I molecules cloaking the malignant cells from recognition by cytotoxic T cells. Targeting HLA downregulation of neuroblastoma cells in combination with engineered tumor specific T cells for the treatment of high-risk neuroblastoma can be helpful.

### 2. Experiments Were Designed To Determine The Cytolytic Activity of TCR-Transduced T Cells Specific for CGA Against Neuroblastoma Cells Pretreated With IFNγ, Provided Either as a Recombinant Protein or Released By Cytokine Secreting Cells

The goal of this project is to develop a therapeutic approach to improve survival and cure rates for patients with high-risk neuroblastoma. The failure to salvage half of the patients with high-risk neuroblastoma is concerning, supporting investigation of novel regimens in this group of patients.

i. Cancer-Germline Antigens NY-ESO-1 and PRAME are Suitable Targets For The Immunotherapy of Neuroblastoma

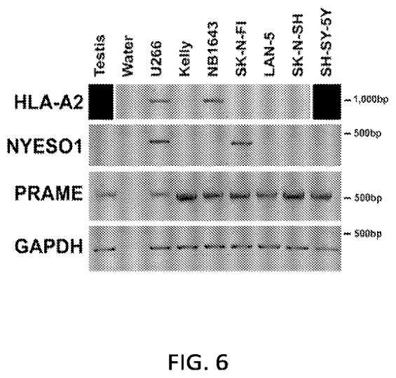

NY-ESO-1 and PRAME are the most frequently found CGA in neuroblastoma. NY-ESO-1 is expressed in 23-82% of patients as determined by immunohistochemistry, and PRAME is expressed in 93% of patients as determined by RT-PCR. PRAME was previously found to be significantly expressed in high-risk neuroblastoma. It was recently shown that all of the neuroblastoma cell lines express PRAME mRNA, while NY-ESO-1 is positive in only one of our neuroblastoma cell lines (FIG. 6). In addition, we determined the presence of the HLA-A2 allele in our neuroblastoma cell lines, with only NB1643 being positive. The adoptive transfer of genetically modified CGA-specific T cells is a promising therapeutic approach, however, particularly in neuroblastoma, without intervention, endogenous HLA levels appear to be too low to stimulate T cell mediated anti-tumor attack.

ii. MHC I Induction on Neuroblastoma Cell Lines Using IFNγ as a Recombinant Protein

Based on previous reports, treatment with IFNγ can enhance the functional avidity of TCR-transduced T cells and can improve antitumor responses in clinical studies. The current data show that IFNγ, when provided as a recombinant protein, induced the upregulation of HLA class I (ABC) on all tested neuroblastoma cell lines (FIG. 1). Importantly, IFNγ upregulated HLA-A2 on neuroblastoma cell line NB1643.

iii. Cytotoxic Activity of NY-ESO1-Specific TCR-Transduced T Cells

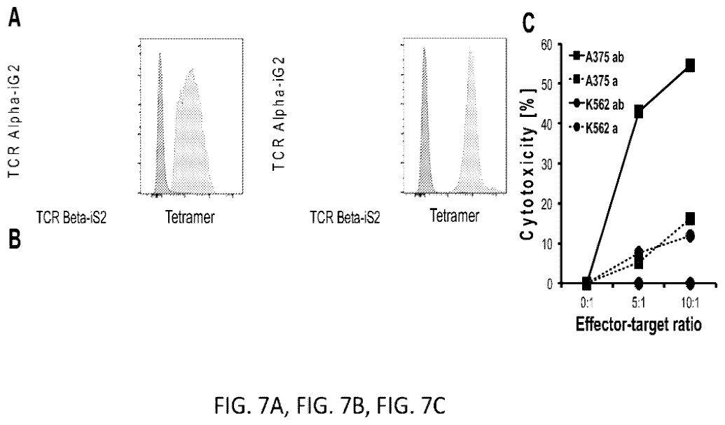

Primary human T cells were generated expressing a TCR recognizing NY-ESO-1. Using sequential transduction of alpha/beta chains allowing prior knockout of individual endogenous chains using TALEN technology and CD3 sorting efficient transduction using fluorescent reporter genes expressed in tandem with the respective novel TCR chain was demonstrated (FIG. 7A). Staining with an HLA-A2/NY-ESO-1 tetramer strong expression of the transgenic receptor on the surface of T cells was shown (FIG. 7A). After stimulation with autologous antigen-presenting cells loaded with the NY-ESO-1 peptide specific secretion of IFNγ by ELISpot was observed (FIG. 7B). Importantly, the knockout of the endogenous TCR improved both the surface expression and the response to peptide-loaded antigen-presenting cells. Finally, using a flow cytometry-based cytotoxicity assay, it was demonstrated that the fully reprogrammed TCR-transduced T cells, but not T cells expressing only the novel receptor's alpha chain, efficiently kill HLA-A2-positive A375 melanoma cells expressing NY-ESO-1 (FIG. 7C).

### 3. Experimental Design and Methods

i. Aim 1: Determine the Ability of Pretreatment with Recombinant IFNγ and T Cells Engineered to Secrete IFNγ to Upregulate HLA Class I Expression In Neuroblastoma ell Lines
a. Induction of HLA Expression in Neuroblastoma Cells Using Recombinant IFNγ

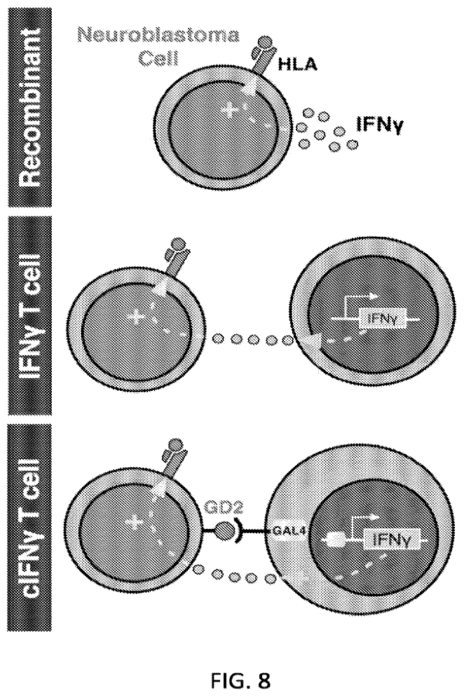

In order to develop a strategy to induce HLA class I, neuroblastoma cell lines can be cultured in the presence of interferon gamma IFNγ, provided as a recombinant protein (FIG. 8). After 24 hours of treatment the samples can be analyzed by flow cytometry and quantitative RT-PCR. HLA class I, HLA class II and levels of coinhibitory ligands expressed on the surface of neuroblastoma cells will be determined. Subsequently, an IFNγ titration experiment can be performed, to determine the minimal IFNγ concentration needed to upregulate HLA class I. A time-course experiment can also be performed to determine the stability of HLA class I expression after removing exogenous IFNγ from the culture.

b. Induction of HLA Expression By T Cells Engineered to Secrete IFNγ Constitutively

To increase the specificity of delivering IFNγ to the malignant cells IFNγ secreting T cells can be developed (FIG. 8). Using lentiviral gene transfer bulk T cell populations expressing IFNγ constitutively can be generated. Individual T cell population can then be sorted by FACS (naïve, central memory, effector memory, effector) and IFNγ levels secreted by different T cell populations determined by ELISA. How T cell phenotypes change due to constitutive IFNγ expression in the absence of a coordinated T cell activation program can be determined by flow cytometry, and whether exhaustion markers such as TIM-3, LAG-3 or PD-1 are upregulated over time. In addition, it can be determined whether expression of IFNγ upregulates expression of other T cell-associated cytokines, including GM-CSF, IL-2, IL-4, IL-5, IL-6, IL-7, IL-8, IL-10, IL-12, IL-13, IL-17A, IL-21, and IL-23 using Luminex technology. Finally, the ability of these cells to induce HLA upregulation in neuroblastoma cells during co-culture assays as determined by flow cytometry and quantitative RT-PCR can be determined.

c. Development of T Cells Conditionally Expressing IFNγ When Encountering Target Cells Expressing GD2

To improve the delivery of IFNγ to neuroblastoma cells a chimeric receptor comprised of a single-chain antibody domain specific for GD2, a major ganglioside expressed on the surface of human neuroblastoma cells, the minimal Notch transmembrane domain, as well as the transcriptional activator GAL4-VP64 can be developed. In combination with an IFNγ expression cassette carrying response elements for GAL4-VP64 previously engineered into the same T cells, this conditional IFNγ (cIFNγ) construct can allow the secretion of IFNγ only in the presence of cells expressing GD2. scFv domains have been developed against various surface antigens, such as CD19, and extensively validated these constructs in terms of target specificity as well as cytotoxic activity when expressed as activating chimeric antigen receptors. cIFNγ construct can be established using a CD19 scFv expressed in primary T cells in co-culture assays with K562 chronic myeloid leukemia cells engineered to express CD19 or parental CD19-negative K562 cells and determine the levels of secreted IFNγ by ELISA. The CD19 scFv can be switched to a previously described GD2-specific scFv, and repeat co-culture experiments with GD2-positive and GD2-negative neuroblastoma cells and determine IFNγ levels by ELISA and HLA expression levels by flow cytometry. A time-course experiment determining HLA levels can be performed after removing IFNγ-expressing cells from the culture. Finally, whether adding low numbers of GD2-positive cells to otherwise GD2-negative neuroblastoma cell cultures together with cIFNγ cells increases HLA expression levels on all present neuroblastoma cells and not just on GD2-positive cells can be determined.

ii. Aim 2: Determine the Cytolytic Activity of TCR-Transduced T Cells Specific For CGA Against Neuroblastoma Cells Pretreated with IFNγ, Provided Either as a Recombinant Protein or Released by Cytokine Secreting Cells
a. Determine in Vitro Cytotoxicity of CGA-Specific TCR-Transduced T Cells Against Neuroblastoma Cell Lines

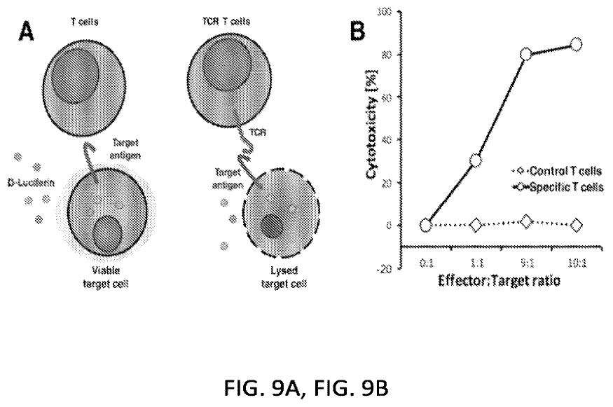

NY-ESO-1-specific TCR-transgenic T cells have been established. Neuroblastoma cell lines express the common tumor antigens NY-ESO-1 and PRAME and their expression in primary neuroblastoma from high-risk patients can be determined by immunohistochemistry. Unfortunately, while one of the cell lines, NB1643, is positive for HLA-A2 and expresses PRAME it does not express NY-ESO-1. Therefore, these cells can be transduced to stably overexpress NY-ESO-1. The ability of NY-ESO-1-and PRAME-specific transgenic T cells to target HLA-A2-positive neuroblastoma cell lines showing or lacking expression of the respective antigen using a luciferase-based cytotoxicity assay can be determined (FIG. 9A). As a control melanoma cell lines can be used which we have previously successfully targeted using these TCR-transduced T cells (FIG. 9B). In a next step whether pretreatment of neuroblastoma cell lines with recombinant IFNγ or IFNγ produced by cytokine-secreting cells enhances neuroblastoma cell killing can be determined. If upregulation of immune checkpoint PD-1 after HLA induction is observed, T cell treatment can be combined with a blocking antibody against PD-1. Finally, whether the presence of low numbers of GD2-positive neuroblastoma cells enhances killing of GD2-positive as well as GD2-negative cells in the same culture can again be determined.

b. Determine in Vivo Activity of TCR-Transduced T Cell After Pretreatment With Recombinant IFNγ or Cells Secreting IFNγ in a Murine Xenograft Model of Neuroblastoma

A patient-derived xenograft model of neuroblastoma in immunocompromised NOD.Cg-PrkdcscidIl2rgtm1Wjl/SzJ mice can be performed. Presence of the HLA-A2 allele can first be confirmed by PCR as well as expression of the respective CGA by immunohistochemistry. The use of NB1643 cells for which we have already confirmed HLA-A2 and CGA expression can also be explored. As previously described, mice can be injected subcutaneously with a neuroblastoma cell line expressing the respective CGA and HLA-A2 suspended in Matrigel. Animals can be treated with systemic recombinant IFNγ as well as cIFNγ cells by tail vein or intratumoral injection and determine HLA as well as PD-L1 expression levels before and after treatment by flow cytometry and quantitative RT-PCR. In case upregulation of HLA class I is observed in vivo animals can be treated with CGA-specific TCR-transduced T cells and tumor growth determined by caliper measurements and bioluminescence imaging, and toxicities.

### 4. Results and Potential Clinical Relevance

HLA class I is upregulated in response to IFNγ treatment and it has previously been shown that natural killer cells are able to induce HLA expression on neuroblastoma cells in an IFNγ dependent manner. Substantial upregulation of HLA class I in response to pretreatment with constitutively cytokine-secreting T cells can be achieved. In addition, it has previously been shown that synNotch receptors can be used to drive T cell phenotype and function, GD2-specific T cells have been shown to be effective in the preclinical setting, and various CAR T cell constructs with potent anti-tumor activity have been generated. T cells conditionally expressing IFNγ can be generated in response to engagement of GD2. Furthermore, it has been previously shown that the NY-ESO-1-specific TCR-transgenic T cells are effective against melanoma cells in vitro and it has been shown that neuroblastoma cells can be targeted using this approach. The HLA induction approach can enhance efficacy and the conditional INFγ secretion approach can have substantial impact on future adoptive T cell therapies, in particular when targeting solid malignancies by enabling a safer and targeted delivery of INFγ to upregulate HLA class I in this setting.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Cornetta K, Berebitsky D, Behnia M, Traycoff C, Srour EF, Sledge GW. A retroviral vector expressing human interferon gamma upregulates MHC antigen expression in human breast cancer and leukemia cell lines. Cancer Gene Ther. 1994;1(2):91-8.
2. Martini M, Testi MG, Pasetto M, Picchio MC, Innamorati G, Mazzocco M, et al. IFN-gamma-mediated upmodulation of MHC class I expression activates tumor-specific immune response in a mouse model of prostate cancer. Vaccine. 2010;28(20):3548-57.
3. Street D, Kaufmann AM, Vaughan A, Fisher SG, Hunter M, Schreckenberger C, et al. Interferon-gamma enhances susceptibility of cervical cancer cells to lysis by tumor-specific cytotoxic T cells. Gynecol Oncol. 1997;65(2):265-72.
4. Chang AY, Dao T, Gejman RS, Jarvis CA, Scott A, Dubrovsky L, et al. A therapeutic T cell receptor mimic antibody targets tumor-associated PRAME peptide/HLA-I antigens. J Clin Invest. 2017;127(7):2705-18.
5. Hallermalm K, Seki K, Wei C, Castelli C, Rivoltini L, Kiessling R, et al. Tumor necrosis factor-alpha induces coordinated changes in major histocompatibility class I presentation pathway, resulting in increased stability of class I complexes at the cell surface. Blood. 2001;98(4):1108-15.
6. Alberts DS, Marth C, Alvarez RD, Johnson G, Bidzinski M, Kardatzke DR, et al. Randomized phase 3 trial of interferon gamma-1b plus standard carboplatin/paclitaxel versus carboplatin/paclitaxel alone for first-line treatment of advanced ovarian and primary peritoneal carcinomas: results from a prospectively designed analysis of progression-free survival. Gynecol Oncol. 2008;109(2):174-81.
7. Giannopoulos A, Constantinides C, Fokaeas E, Stravodimos C, Giannopoulou M, Kyroudi A, et al. The immunomodulating effect of interferon-gamma intravesical instillations in preventing bladder cancer recurrence. Clin Cancer Res. 2003;9(15):5550-8.
8. Windbichler GH, Hausmaninger H, Stummvoll W, Graf AH, Kainz C, Lahodny J, et al. Interferon-gamma in the first-line therapy of ovarian cancer: a randomized phase III trial. Br J Cancer. 2000;82(6):1138-44.
9. Raffaghello L, Prigione I, Bocca P, Morandi F, Camoriano M, Gambini C, et al. Multiple defects of the antigen-processing machinery components in human neuroblastoma: immunotherapeutic implications. Oncogene. 2005;24(29):4634-44
10. Spel L, Boelens JJ, van der Steen DM, Blokland NJ, van Noesel MM, Molenaar JJ, et al. Natural killer cells facilitate PRAME-specific T-cell reactivity against neuroblastoma. Oncotarget. 2015;6(34):35770-81.
11. Irwin MS, Park JR. Neuroblastoma: paradigm for precision medicine. Pediatr Clin North Am. 2015;62(1):225-56.
12. Yu AL, Gilman AL, Ozkaynak MF, London WB, Kreissman SG, Chen HX, et al. Anti-GD2 antibody with GM-CSF, interleukin-2, and isotretinoin for neuroblastoma. The New England journal of medicine. 2010;363(14):1324-34.
13. Speleman F, Park JR, Henderson TO. Neuroblastoma: A Tough Nut to Crack. Am Soc Clin Oncol Educ Book. 2016;35:e548-57.
14. Singh N, Kulikovskaya I, Barrett DM, Binder-Scholl G, Jakobsen B, Martinez D, et al. T cells targeting NY-ESO-1 demonstrate efficacy against disseminated neuroblastoma. Oncoimmunology. 2016;5(1):e1040216.
15. Haworth KB, Leddon JL, Chen CY, Horwitz EM, Mackall CL, Cripe TP. Going back to class I: MHC and immunotherapies for chiood cancer. Pediatr Blood Cancer. 2015;62(4):571-6.
16. Zhang S, Kohli K, Black RG, Yao L, Spadinger SM, He Q, et al. Systemic Interferon-gamma Increases MHC Class I Expression and T-cell Infiltration in Cold Tumors: Results of a Phase 0 Clinical Trial. Cancer immunology research. 2019.
17. Garcia-Diaz A, Shin DS, Moreno BH, Saco J, Escuin-Ordinas H, Rodriguez GA, et al. Interferon Receptor Signaling Pathways Regulating PD-L1 and PD-L2 Expression. Cell Rep. 2017;19(6):1189-201.
18. Mimura K, Teh JL, Okayama H, Shiraishi K, Kua LF, Koh V, et al. PD-L1 expression is mainly regulated by interferon gamma associated with JAK-STAT pathway in gastric cancer. Cancer Sci. 2018;109(1):43-53.
19. Amir AL, van der Steen DM, van Loenen MM, Hagedoorn RS, de Boer R, Kester MD, et al. PRAME-specific Allo-HLA-restricted T cells with potent antitumor reactivity useful for therapeutic T-cell receptor gene transfer. Clin Cancer Res. 2011;17(17):5615-25.
20. Scholtalbers J, Boegel S, Bukur T, Byl M, Goerges S, Sorn P, et al. TCLP: an online cancer cell line catalogue integrating HLA type, predicted neo-epitopes, virus and gene expression. Genome Med. 2015;7:118.
21. Mauldin IS, Wages NA, Stowman AM, Wang E, Smolkin ME, Olson WC, et al. Intratumoral interferon-gamma increases chemokine production but fails to increase T cell infiltration of human melanoma metastases. Cancer Immunol Immunother. 2016;65(10):1189-99.

We claim:

1. A nucleic acid construct comprising:
a promoter;
a nucleic acid sequence encoding a single-chain variable fragment (scFv);
a nucleic acid sequence encoding a notch transmembrane domain; and
a nucleic acid sequence encoding a transcription factor.

2. The nucleic acid construct of claim 1, wherein the promoter is a constitutive promoter.

3. The nucleic acid construct of claim 1, wherein the promoter is a regulatable promoter.

4. The nucleic acid construct of any one of claims 1-3, wherein the scFv is a neuroblastoma-specific ScFv.

5. The nucleic acid construct of any one of claims 1-4, wherein the scFv is a GD2 specific ScFv.

6. The nucleic acid construct of any one of claims 1-4, wherein the scFv is a CD19 specific ScFv.

7. The nucleic acid construct of any one of claims 1-6, wherein the scFv comprises a heavy chain fragment and a light chain fragment.

8. The nucleic acid construct of claim 7, wherein the heavy chain fragment comprises an amino acid sequence of
QVKLQQSGPELVEPGASVKISCKTSGYKFTEYTMHWVKQSHGKSL
EWIGGINPNNGGTNYKQKFKGKATLTVDKSSSTAYMELRSLTSED
SAVYYCARDTTVPFAYWVQGTTVTVSS;
and
wherein the light chain fragment comprises an amino acid sequence of
DIELTQSPAIMSASPGEKVTMTCSGSSSISYMHWYQQKPVTSPKR
WIYDTSKLASGVPARFSGSGSGTSYSLTISSMEAVDAATYYCHQR
SSYPLTFGAGTQLEIKR.

9. The nucleic acid construct of claim 7, wherein the heavy chain fragment comprises an amino acid sequence of
EVQLVQSGAEVEKPGASVKISCKASGSSFTGYNMNWVRQNIGKSL
EWIGAIDPYYGGTSYNQKFKGRATLTVDKSTSTAYMHLKSLRSED
TAVYYCVSGMEYWGQGTSVTVSS;
and
wherein the light chain fragment comprises an amino acid sequence of
DVVMTQTPLSLPVTPGEPASISCRSSQSLVHRNGNTYLHWYLQKP
GQSPKLLIHKVSNRFSGVPDRFSGSGSGTDFTLKISRVEAEDLGV
YFCSQSTHVPPLTFGAGTKLELKR.

10. The nucleic acid construct of claim 7, wherein the heavy chain fragment comprises an amino acid sequence of
QVQLVESGPGVVQPGRSLRISCAVSGFSVTNYGVHWVRQPPGKGL
EWLGVIWAGGITNYNSAFMSRLTISKDNSKNTVYLQMNSLRAEDT
AMYYCASRGGHYGYALDYWGQGTLVTVSS;
and
wherein the light chain fragment comprises an amino acid sequence of
EIVMTQTPATLSVSAGERVTITCKASQSVSNDVTWYQQKPGQAPRLL
IYSASNRYSGVPARFSGSGYGTEFTFTISSVQSEDFAVYFCQQDYSS
FGQGTKLEIKR.

11. The nucleic acid construct of claim 7, wherein the heavy chain fragment comprises an amino acid sequence of
QVQLQESGPGLVKPSQTLSITCTVSGFSLASYNIHWVRQPPGKGLEW
LGVIWAGGSTNYNSALMSRLTISKDNSKNQVFLKMSSLTAADTAVYY
CAKRSDDYSWFAYWGQGTLVTVSS;
and
wherein the light chain fragment comprises an amino acid sequence of
ENQMTQSPSSLSASVGDRVTMTCRASSSVSSSYLHWYQQKSGKAPKV
WIYSTSNLASGVPSRFSGSGSGTDYTLTISSLQPEDFATYYCQQYSG
YPITFGQGTKVEIKR.

12. The nucleic acid construct of claim 7, wherein the heavy chain fragment comprises an amino acid sequence of
LISEEDLEVQLVETGGGVVKPGGSLRLSCAASGFTFSDYYMSWIRQA
PGKGLEWVSYISSSGSTIYYADSVKGRFTISRDNSKNTLYLQMNSLR
AEDTAVYYCARESGYDYVFDYWGQGTLVAVSS;
and
wherein the light chain fragment comprises an amino acid sequence of
DIQMTQSPSTLSAFVGDRVTITCRASQSISSWLAWYQQKPGKAPKLL
IYAASTLQSGVPSRFSGSGSGTEFTLTISSLQPEDFATYYCQQLNSY
PITFGQGTRLEIKRILDYSF.

13. The nucleic acid construct of claim 7, wherein the heavy chain fragment comprises an amino acid sequence of
LISEEDLEVQLVESGGGLVQPGGSLRLSCAASGFTFSSFGMHWVRQA
PGKGLEWVAYISSDSSAIYYADTVKGRFTISRDNAKNSLYLQMNSLR
DEDTAVYYCGRGRENIYYGSRLDYWGQGTTVTVSS;
and
wherein the light chain fragment comprises an amino acid sequence of
DIQLTQSPSFLSASVGDRVTITCKASQNVDTNVAWYQQKPGKAPKAL
IYSASYRYSGVPSRFSGSGSGTDFTLTISSLQPEDFATYYCQQYNNY
PFTFGQGTKLEIKILDYSF.

14. The nucleic acid construct of claim 7, wherein the heavy chain fragment comprises an amino acid sequence of
LISEEDLQVQLQQPGAELVKPGASVKLSCKASGYTFTGYWMHWVKQR
PGHGLEWIGEINPSNGRTNYNERFKSKATLTVDKSSTTAFMQLSGLT
SEDSAVYFCARDYYGTSYNFDYWGQGTTLTVSS;
and
wherein the light chain fragment comprises an amino acid sequence of
DIQMTQSSSSFSVSLGDRVTITCKANEDINNRLAWYQQTPGNSPRLL
ISGATNLVTGVPSRFSGSGSGKDYTLTITSLQAEDFATYYCQQYWST
PFTFGSGTELEIKVEILDYSF.

15. The nucleic acid construct of any one of claims 1-14, wherein the heavy chain fragment and light chain fragment are joined via a linker.

16. The nucleic acid construct of claim 15, wherein the linker comprises an amino acid sequence of GGGGSGGGGSGGGGS or GGGGSGGGGSGGGGSGGGGS.

17. The nucleic acid construct of any one of claims 1-16, wherein the notch transmembrane domain comprises the amino acid sequence of
ILDYSFTGGAGRDIPPPQIEEACELPECQVDAGNKVCNLQCNNHACG
WDGGDCSLNFNDPWKNCTQSLQCWKYFSDGHCDSQCNSAGCLFDGFD
CQLTEGQCNPLYDQYCKDHFSDGHCDQGCNSAECEWDGLDCAEHVPE
RLAAGTLVLVVLLPPDQLRNNSFHFLRELSHVLHTNVVFKRDAQGQQ
MIFPYYGHEEELRKHPIKRSTVGWATSSLLPGTSGGRQRRELDPMDI
RGSIVYLEIDNRQCVQSSSQCFQSATDVAAFLGALASLGSLNIPYKI
EAVKSEPVEPPLPSQLHLMYVAAAAFVLLFFVGCGVLLSRKRRR.

18. The nucleic acid construct of any one of claims 1-17, wherein the notch transmembrane domain further comprises one or more EGF repeats (ERR) from the notch extracellular domain.

19. The nucleic acid construct of claim 18, wherein the ERR sequence comprises the amino acid sequence of PCVGSNPCYNQGTCEPTSENPFYRCLCPAKFNGLLCH.

20. The nucleic acid construct of any one of claims 1-19, wherein the transcription factor is a transcription activator.

21. The nucleic acid construct of claim 20, wherein the transcription activator comprises Gal4.

22. The nucleic acid construct of claim 21, wherein the transcription activator comprises a Gal4-VP16 fusion protein.

23. The nucleic acid construct of claim 22, wherein the Gal4-VP64 fusion protein comprises the amino acid sequence of
MKLLSSIEQACDICRLKKLKCSKEKPKCAKCLKNNWECRYSPKTKRS
PLTRAHLTEVESRLERLEQLFLLIFPREDLDMILKMDSLQDIKALLT
GLFVQDNVNKDAVTDRLASVETDMPLTLRQHRISATSSSEESSNKGQ
RQLTVSAAAGGSGGSGGSDALDDFDLDMLGSDALDDFDLDMLGSDAL
DDFDLDMLGSDALDDFDLDMLGS.

24. The nucleic acid construct of any one of claims 1-23, further comprising a nucleic acid sequence encoding a detection agent.

25. The nucleic acid construct of claim 24, wherein the detection agent allows for visual detection or purification.

26. The nucleic acid construct of claim 23, wherein the detection agent is a myc tag, his tag, fluorescent tag, FLAG tag, or hemagglutinin tag.

27. A nucleic acid construct comprising the sequence of
ATGGCCCTGCCTGTTACAGCTCTGCTGCTGCCTCTGGCTCTGCTTCT
GCATGCCGCTAGACCTGAGATCGTGATGACACAGACCCCTGCCACAC
TGTCTGTGTCTGCCGGCGAGAGAGTGACCATTACCTGCAAGGCCAGC
CAGAGCGTGTCCAACGACGTGACCTGGTATCAGCAGAAGCCAGGACA
GGCCCCTCGGCTGCTGATCTACAGCGCCAGCAATAGATACAGCGGCG
TGCCCGCCAGATTTTCCGGCTCTGGATACGGCACCGAGTTCACCTTC
ACCATCAGCTCCGTGCAGAGCGAGGACTTCGCTGTGTACTTCTGTCA
GCAAGACTACAGCTCCTTCGGCCAGGGCACCAAGCTGGAAATCAAGA
GAGGAGGCGGAGGATCTGGTGGCGGAGGAAGTGGCGGAGGCGGTTCT
GGCGGTGGTGGATCTGAGCAGAAGCTGATCTCCGAAGAGGACCTCCA
GGTGCAGCTGGTGGAATCTGGACCTGGTGTTGTGCAGCCTGGCAGAA
GCCTGAGAATCAGCTGTGCCGTGTCCGGCTTCAGCGTGACCAATTAT
GGCGTGCACTGGGTCCGACAGCCTCCAGGCAAAGGACTGGAATGGCT
GGGAGTGATTTGGGCTGGCGGCATCACCAACTACAACAGCGCCTTCA
TGAGCCGGCTGACCATCAGCAAGGACAACAGCAAGAACACCGTGTAC
CTGCAGATGAACAGCCTGAGAGCCGAGGACACCGCCATGTACTACTG
TGCTTCTAGAGGCGGCCACTACGGCTACGCCCTGGATTATTGGGGAC
AGGGCACACTGGTCACAGTGTCTAGCCCTTGCGTGGGCAGCAACCCC
TGCTACAATCAGGGCACATGCGAGCCCACCAGCGAGAACCCCTTCTA
CAGATGTCTGTGCCCCGCCAAGTTCAACGGCCTGCTGTGTCACATCC
TGGACTACAGCTTTACCGGCGGAGCCGGCAGAGATATCCCTCCACCT
CAGATTGAGGAAGCCTGCGAGCTGCCTGAGTGTCAGGTTGACGCCGG
CAACAAAGTGTGCAACCTGCAGTGCAACAACCACGCCTGTGGATGGG
ATGGCGGCGACTGTAGCCTGAACTTCAACGACCCCTGGAAGAACTGC
ACCCAGAGCCTGCAGTGTTGGAAGTACTTTAGCGACGGCCACTGCGA
CAGCCAGTGTAATTCTGCCGGATGCCTGTTCGACGGCTTCGACTGCC
AACTGACAGAGGGCCAGTGCAACCCTCTGTACGACCAGTACTGCAAG
GACCACTTCTCCGATGGCCACTGTGACCAGGGCTGTAATAGCGCCGA
GTGCGAGTGGGATGGACTGGATTGTGCCGAGCACGTGCCAGAAAGAC
TGGCCGCTGGAACACTGGTGCTGGTGGTTCTTCTGCCTCCTGACCAG
CTGCGGAACAACAGCTTCCACTTCCTGCGGGAACTGAGCCACGTGCT
GCACACCAACGTGGTGTTCAAGAGAGATGCCCAGGGACAGCAGATGA
TCTTCCCCTACTACGGCCACGAAGAGGAACTGCGGAAGCACCCCATC
AAGAGATCTACAGTCGGCTGGGCCACCTCCAGTCTGCTGCCTGGAAC
AAGTGGCGGCAGACAGAGAAGAGAACTGGACCCCATGGACATCCGGG
GCAGCATCGTGTACCTGGAAATCGACAACCGGCAGTGCGTGCAGAGC
AGCTCCCAGTGTTTTCAGAGCGCTACTGACGTGGCCGCCTTTCTGGG
AGCACTTGCTTCTCTGGGCAGCCTGAACATCCCCTACAAGATCGAGG
CCGTGAAGTCCGAGCCTGTGGAACCTCCTCTGCCTTCTCAGCTGCAC
CTTATGTACGTGGCAGCCGCCGCTTTCGTGCTGCTGTTCTTTGTTGG
ATGCGGAGTGCTGCTGAGCCGGAAGCGGAGAAGAATGAAGCTGCTGT
CCAGCATCGAGCAGGCCTGTGACATCTGCAGACTGAAGAAACTGAAG
TGCAGCAAAGAAAAGCCCAAGTGCGCCAAGTGCCTGAAGAACAATTG
GGAGTGCCGGTACAGCCCCAAGACCAAGAGATCCCCTCTGACAAGAG
CCCACCTGACCGAGGTGGAAAGCCGGCTGGAAAGACTCGAGCAGCTG
TTCCTGCTGATCTTTCCACGCGAGGACCTGGACATGATTCTGAAGAT
GGACTCTCTGCAGGACATCAAGGCCCTGCTGACCGGCCTGTTCGTGC
AGGACAACGTGAACAAGGACGCCGTGACCGATAGACTGGCCTCCGTG
GAAACCGACATGCCCCTGACACTGAGACAGCACAGAATCAGCGCCAC
CAGCAGCAGCGAGGAAAGCAGCAACAAGGGCCAGAGACAGCTGACAG
TGTCTGCTGCAGCTGGCGGATCAGGTGGTAGTGGCGGATCTGATGCC
CTGGACGACTTTGACCTGGATATGCTGGGCAGCGACGCCCTGGATGA
TTTTGATCTGGACATGCTCGGCTCCGACGCTCTCGACGATTTCGACC
TCGACATGTTGGGATCCGACGCACTTGATGACTTCGATCTCGATATG
CTCGGGTCCTGA.

28. A nucleic acid construct comprising the sequence of
CGATACCGTCGACCAAGGCAGCTGTAGATCTTAGCCACTTTTTAAAA
GAAAAGGGGGGACTGGAAGGGCTAATTCACTCCCAACGAAGACAAGA
TCTGCTTTTTGCTTGTACTGGGTCTCTCTGGTTAGACCAGATCTGAG
CCTGGGAGCTCTCTGGCTAACTAGGGAACCCACTGCTTAAGCCTCAA
TAAAGCTTGCCTTGAGTGCTTCAAGTAGTGTGTGCCCGTCTGTTGTG
TGACTCTGGTAACTAGAGATCCCTCAGACCCTTTTAGTCAGTGTGGA
AAATCTCTAGCAGCATCTAGAATTAATTCCGTGTATTCTATAGTGTC
ACCTAAATCGTATGTGTATGATACATAAGGTTATGTATTAATTGTAG
CCGCGTTCTAACGACAATATGTACAAGCCTAATTGTGTAGCATCTGG
CTTACTGAAGCAGACCCTATCATCTCTCTCGTAAACTGCCGTCAGAG
TCGGTTTGGTTGGACGAACCTTCTGAGTTTCTGGTAACGCCGTCCCG
CACCCGGAAATGGTCAGCGAACCAATCAGCAGGGTCATCGCTAGCCA
GATCCTCTACGCCGGACGCATCGTGGCCGGCATCACCGGCGCCACAG
GTGCGGTTGCTGGCGCCTATATCGCCGACATCACCGATGGGGAAGAT
CGGGCTCGCCACTTCGGGCTCATGAGCGCTTGTTTCGGCGTGGGTAT
GGTGGCAGGCCCCGTGGCCGGGGGACTGTTGGGCGCCATCTCCTTGC
ATGCACCATTCCTTGCGGCGGCGGTGCTCAACGGCCTCAACCTACTA
CTGGGCTGCTTCCTAATGCAGGAGTCGCATAAGGGAGAGCGTCGAAT
GGTGCACTCTCAGTACAATCTAGCTCTGATGCCGCATAGTTAAGCCA
GCCCCGACACCCGCCAACACCCGCTGACGCGCCCTGACGGGCTTGTC
TGCTCCCGGCATCCGCTTACAGACAAGCTGTGACCGTCTCCGGGAGC
TGCATGTGTCAGAGGTTTTCACCGTCATCACCGAAACGCGCGAGACG
AAAGGGCCTCGTGATACGCCTATTTTTATAGGTTAATGTCATGATAA
TAATGGTTTCTTAGACGTCAGGTGGCACTTTTCGGGGAAATGTGCGC
GGAACCCCTATTTGTTTATTTTTCTAAATACATTCAAATATGTATCC
GCTCATGAGACAATAACCCTGATAAATGCTTCAATAATATTGAAAAA
GGAAGAGTATGAGTATTCAACATTTCCGTGTCGCCCTTATTCCCTTT
TTTGCGGCATTTTGCCTTCCTGTTTTTGCTCACCCAGAAACGCTGGT
GAAAGTAAAAGATGCTGAAGATCAGTTGGGTGCACGAGTGGGTTACA
TCGAACTGGATCTCAACAGCGGTAAGATCCTTGAGAGTTTTCGCCCC
GAAGAACGTTTTCCAATGATGAGCACTTTTAAAGTTCTGCTATGTGG
CGCGGTATTATCCCGTATTGACGCCGGGCAAGAGCAACTCGGTCGCC
GCATACACTATTCTCAGAATGACTTGGTTGAGTACTCACCAGTCACA
GAAAAGCATCTTACGGATGGCATGACAGTAAGAGAATTATGCAGTGC
TGCCATAACCATGAGTGATAACACTGCGGCCAACTTACTTCTGACAA
CGATCGGAGGACCGAAGGAGCTAACCGCTTTTTTGCACAACATGGGG
GATCATGTAACTCGCCTTGATCGTTGGGAACCGGAGCTGAATGAAGC
CATACCAAACGACGAGCGTGACACCACGATGCCTGTAGCAATGGCAA
CAACGTTGCGCAAACTATTAACTGGCGAACTACTTACTCTAGCTTCC
CGGCAACAATTAATAGACTGGATGGAGGCGGATAAAGTTGCAGGACC
ACTTCTGCGCTCGGCCCTTCCGGCTGGCTGGTTTATTGCTGATAAAT
CTGGAGCCGGTGAGCGTGGGTCTCGCGGTATCATTGCAGCACTGGGG
CCAGATGGTAAGCCCTCCCGTATCGTAGTTATCTACACGACGGGGAG
TCAGGCAACTATGGATGAACGAAATAGACAGATCGCTGAGATAGGTG
CCTCACTGATTAAGCATTGGTAACTGTCAGACCAAGTTTACTCATAT
ATACTTTAGATTGATTTAAAACTTCATTTTTAATTTAAAAGGATCTA
GGTGAAGATCCTTTTTGATAATCTCATGACCAAAATCCCTTAACGTG
AGTTTTCGTTCCACTGAGCGTCAGACCCCGTAGAAAAGATCAAAGGA
TCTTCTTGAGATCCTTTTTTTCTGCGCGTAATCTGCTGCTTGCAAAC
AAAAAAACCACCGCTACCAGCGGTGGTTTGTTTGCCGGATCAAGAGC
TACCAACTCTTTTTCCGAAGGTAACTGGCTTCAGCAGAGCGCAGATA
CCAAATACTGTCCTTCTAGTGTAGCCGTAGTTAGGCCACCACTTCAA
GAACTCTGTAGCACCGCCTACATACCTCGCTCTGCTAATCCTGTTAC
CAGTGGCTGCTGCCAGTGGCGATAAGTCGTGTCTTACCGGGTTGGAC
TCAAGACGATAGTTACCGGATAAGGCGCAGCGGTCGGGCTGAACGGG
GGGTTCGTGCACACAGCCCAGCTTGGAGCGAACGACCTACACCGAAC
TGAGATACCTACAGCGTGAGCTATGAGAAAGCGCCACGCTTCCCGAA
GGGAGAAAGGCGGACAGGTATCCGGTAAGCGGCAGGGTCGGAACAGG
AGAGCGCACGAGGGAGCTTCCAGGGGGAAACGCCTGGTATCTTTATA
GTCCTGTCGGGTTTCGCCACCTCTGACTTGAGCGTCGATTTTTGTGA
TGCTCGTCAGGGGGGCGGAGCCTATGGAAAAACGCCAGCAACGCGGC
CTTTTTACGGTTCCTGGCCTTTTGCTGGCCTTTTGCTCACATGTTCT
TTCCTGCGTTATCCCCTGATTCTGTGGATAACCGTATTACCGCCTTT
GAGTGAGCTGATACCGCTCGCCGCAGCCGAACGACCGAGCGCAGCGA
GTCAGTGAGCGAGGAAGCGGAAGAGCGCCCAATACGCAAACCGCCTC
TCCCCGCGCGTTGGCCGATTCATTAATGCAGCTGTGGAATGTGTGTC
AGTTAGGGTGTGGAAAGTCCCCAGGCTCCCCAGCAGGCAGAAGTATG
CAAAGCATGCATCTCAATTAGTCAGCAACCAGGTGTGGAAAGTCCCC
AGGCTCCCCAGCAGGCAGAAGTATGCAAAGCATGCATCTCAATTAGT
CAGCAACCATAGTCCCGCCCCTAACTCCGCCCATCCCGCCCCTAACT
CCGCCCAGTTCCGCCCATTCTCCGCCCCATGGCTGACTAATTTTTTT
TATTTATGCAGAGGCCGAGGCCGCCTCGGCCTCTGAGCTATTCCAGA
AGTAGTGAGGAGGCTTTTTTGGAGGCCTAGGCTTTTGCAAAAAGCTT
GGACACAAGACAGGCTTGCGAGATATGTTTGAGAATACCACTTTATC
CCGCGTCAGGGAGAGGCAGTGCGTAAAAAGACGCGGACTCATGTGAA
ATACTGGTTTTTAGTGCGCCAGATCTCTATAATCTCGCGCAACCTAT
TTTCCCCTCGAACACTTTTTAAGCCGTAGATAAACAGGCTGGGACAC
TTCACATGAGCGAAAAATACATCGTCACCTGGGACATGTTGCAGATC
CATGCACGTAAACTCGCAAGCCGACTGATGCCTTCTGAACAATGGAA
AGGCATTATTGCCGTAAGCCGTGGCGGTCTGTACCGGGTGCGTTACT
GGCGCGTGAACTGGGTATTCGTCATGTCGATACCGTTTGTATTTCCA
GCTACGATCACGACAACCAGCGCGAGCTTAAAGTGCTGAAACGCGCA
GAAGGCGATGGCGAAGGCTTCATCGTTATTGATGACCTGGTGGATAC
CGGTGGTACTGCGGTTGCGATTCGTGAAATGTATCCAAAAGCGCACT
TTGTCACCATCTTCGCAAAACCGGCTGGTCGTCCGCTGGTTGATGAC
TATGTTGTTGATATCCCGCAAGATACCTGGATTGAACAGCCGTGGGA
TATGGGCGTCGTATTCGTCCCGCCAATCTCCGGTCGCTAATCTTTTC
AACGCCTGGCACTGCCGGGCGTTGTTCTTTTTAACTTCAGGCGGGTT
ACAATAGTTTCCAGTAAGTATTCTGGAGGCTGCATCCATGACACAGG
CAAACCTGAGCGAAACCCTGTTCAAACCCCGCTTTAAACATCCTGAA
ACCTCGACGCTAGTCCGCCGCTTTAATCACGGCGCACAACCGCCTGT
GCAGTCGGCCCTTGATGGTAAAACCATCCCTCACTGGTATCGCATGA
TTAACCGTCTGATGTGGATCTGGCGCGGCATTGACCCACGCGAAATC
CTCGACGTCCAGGCACGTATTGTGATGAGCGATGCCGAACGTACCGA
CGATGATTTATACGATACGGTGATTGGCTACCGTGGCGGCAACTGGA
TTTATGAGTGGGCCCCGGATCTTTGTGAAGGAACCTTACTTCTGTGG
TGTGACATAATTGGACAAACTACCTACAGAGATTTAAAGCTCTAAGG
TAAATATAAAATTTTTAAGTGTATAATGTGTTAAACTACTGATTCTA
ATTGTTTGTGTATTTTAGATTCCAACCTATGGAACTGATGAATGGGA
GCAGTGGTGGAATGCCTTTAATGAGGAAAACCTGTTTTGCTCAGAAG
AAATGCCATCTAGTGATGATGAGGCTACTGCTGACTCTCAACATTCT
ACTCCTCCAAAAAAGAAGAGAAAGGTAGAAGACCCCAAGGACTTTCC
TTCAGAATTGCTAAGTTTTTTGAGTCATGCTGTGTTTAGTAATAGAA
CTCTTGCTTGCTTTGCTATTTACACCACAAAGGAAAAAGCTGCACTG
CTATACAAGAAAATTATGGAAAAATATTCTGTAACCTTTATAAGTAG
GCATAACAGTTATAATCATAACATACTGTTTTTTCTTACTCCACACA
GGCATAGAGTGTCTGCTATTAATAACTATGCTCAAAAATTGTGTACC
TTTAGCTTTTTAATTTGTAAAGGGGTTAATAAGGAATATTTGATGTA
TAGTGCCTTGACTAGAGATCATAATCAGCCATACCACATTTGTAGAG
GTTTTACTTGCTTTAAAAAACCTCCCACACCTCCCCCTGAACCTGAA
ACATAAAATGAATGCAATTGTTGTTGTTAACTTGTTTATTGCAGCTT
ATAATGGTTACAAATAAAGCAATAGCATCACAAATTTCACAAATAAA
GCATTTTTTTCACTGCATTCTAGTTGTGGTTTGTCCAAACTCATCAA
TGTATCTTATCATGTCTGGATCAACTGGATAACTCAAGCTAACCAAA
ATCATCCCAAACTTCCCACCCCATACCCTATTACCACTGCCAATTAC
CTAGTGGTTTCATTTACTCTAAACCTGTGATTCCTCTGAATTATTTT
CATTTTAAAGAAATTGTATTTGTTAAATATGTACTACAAACTTAGTA
GTTGGAAGGGCTAATTCACTCCCAAAGAAGACAAGATATCCTTGATC
TGTGGATCTACCACACACAAGGCTACTTCCCTGATTAGCAGAACTAC
ACACCAGGGCCAGGGGTCAGATATCCACTGACCTTTGGATGGTGCTA
CAAGCTAGTACCAGTTGAGCCAGATAAGGTAGAAGAGGCCAATAAAG
GAGAGAACACCAGCTTGTTACACCCTGTGAGCCTGCATGGGATGGAT
GACCCGGAGAGAGAAGTGTTAGAGTGGAGGTTTGACAGCCGCCTAGC
ATTTCATCACGTGGCCCGAGAGCTGCATCCGGAGTACTTCAAGAACT
GCTGATATCGAGCTTGCTACAAGGGACTTTCCGCTGGGGACTTTCCA
GGGAGGCGTGGCCTGGGCGGGACTGGGGAGTGGCGAGCCCTCAGATC
CTGCATATAAGCAGCTGCTTTTTGCCTGTACTGGGTCTCTCTGGTTA
GACCAGATCTGAGCCTGGGAGCTCTCTGGCTAACTAGGGAACCCACT
GCTTAAGCCTCAATAAAGCTTGCCTTGAGTGCTTCAAGTAGTGTGTG
CCCGTCTGTTGTGTGACTCTGGTAACTAGAGATCCCTCAGACCCTTT
TAGTCAGTGTGGAAAATCTCTAGCAGTGGCGCCCGAACAGGGACTTG
AAAGCGAAAGGGAAACCAGAGGAGCTCTCTCGACGCAGGACTCGGCT
TGCTGAAGCGCGCACGGCAAGAGGCGAGGGGCGGCGACTGGTGAGTA
CGCCAAAAATTTTGACTAGCGGAGGCTAGAAGGAGAGAGATGGGTGC
GAGAGCGTCAGTATTAAGCGGGGGAGAATTAGATCGCGATGGGAAAA
AATTCGGTTAAGGCCAGGGGGAAAGAAAAAATATAAATTAAAACATA
TAGTATGGGCAAGCAGGGAGCTAGAACGATTCGCAGTTAATCCTGGC
CTGTTAGAAACATCAGAAGGCTGTAGACAAATACTGGGACAGCTACA
ACCATCCCTTCAGACAGGATCAGAAGAACTTAGATCATTATATAATA
CAGTAGCAACCCTCTATTGTGTGCATCAAAGGATAGAGATAAAAGAC
ACCAAGGAAGCTTTAGACAAGATAGAGGAAGAGCAAAACAAAAGTAA
GACCACCGCACAGCAAGCGGCCGGTGATCTTCAGACCTGGACGATAT
ATATGAGGGACAATTGGAGAAGTGAATTATATAAATATAAAGTAGTA
AAAATTGAACCATTAGGAGTAGCACCCACCAAGGCAAAGAGAAGAGT
GGTGCAGAGAGAAAAAAGAGCAGTGGGAATAGGAGCTTTGTTCCTTG
GGTTCTTGGGAGCAGCAGGAAGCACTATGGGCGCAGCGTCAATGACG
CTGACGGTACAGGCCAGACAATTATTGTCTGGTATAGTGCAGCAGCA
GAACAATTTGCTGAGGGCTATTGAGGCGCAACAGCATCTGTTGCAAC
TCACAGTCTGGGGCATCAAGCAGCTCCAGGCAAGAATCCTGGCTGTG
GAAAGATACCTAAAGGATCAACAGCTCCTGGGGATTTGGGGTTGCTC
TGGAAAACTCATTTGCACCACTGCTGTGCCTTGGAATGCTAGTTGGA
GTAATAAATCTCTGGAACAGATTTGGAATCACACGACCTGGATGGAG
TGGGACAGAGAAATTAACAATTACACAAGCTTAATACACTCCTTAAT
TGAAGAATCGCAAAACCAGCAAGAAAAGAATGAACAAGAATTATTGG
AATTAGATAAATGGGCAAGTTTGTGGAATTGGTTTAACATAACAAAT
TGGCTGTGGTATATAAAATTATTCATAATGATAGTAGGAGGCTTGGT
AGGTTTAAGAATAGTTTTTGCTGTACTTTCTATAGTGAATAGAGTTA
GGCAGGGATATTCACCATTATCGTTTCAGACCCACCTCCCAACCCCG
AGGGGACCCGACAGGCCCGAAGGAATAGAAGAAGAAGGTGGAGAGAG
AGACAGAGACAGATCCATTCGATTAGTGAACGGATCTCGACGGTCGC
CAAATGGCAGTATTCATCCACAATTTTAAAAGAAAAGGGGGGATTGG
GGGGTACAGTGCAGGGGAAAGAATAGTAGACATAATAGCAACAGACA
TACAAACTAAAGAATTACAAAAACAAATTACAAAAATTCAAAATTTT
CGGGTTTATTACAGGGACAGCAGAGATCCAGTTTGGATCGATAAGCT
TGATATCGAATTGGGTAGGGGAGGCGCTTTTCCCAAGGCAGTCTGGA
GCATGCGCTTTAGCAGCCCCGCTGGGCACTTGGCGCTACACAAGTGG
CCTCTGGCCTCGCACACATTCCACATCCACCGGTAGGCGCCAACCGG
CTCCGTTCTTTGGTGGCCCCTTCGCGCCACCTTCTACTCCTCCCCTA
GTCAGGAAGTTCCCCCCCGCCCCGCAGCTCGCGTCGTGCAGGACGTG
ACAAATGGAAGTAGCACGTCTCACTAGTCTCGTGCAGATGGACAGCA
CCGCTGAGCAATGGAAGCGGGTAGGCCTTTGGGGCAGCGGCCAATAG
CAGCTTTGCTCCTTCGCTTTCTGGGCTCAGAGGCTGGGAAGGGGTGG
GTCCGGGGGCGGGCTCAGGGGCGGGCTCAGGGGCGGGGGGGGCGCCC
GAAGGTCCTCCGGAGGCCCGGCATTCTGCACGCTTCAAAAGCGCACG
TCTGCCGCGCTGTTCTCCTCTTCCTCATCTCCGGGCCTTTCGAATTC
TCACGCGTCAAGTGGAGCAAGGCAGGTGGACAGTGGATCATGGCCCT
GCCTGTTACAGCTCTGCTGCTGCCTCTGGCTCTGCTTCTGCATGCCG
CTAGACCTGAGATCGTGATGACACAGACCCCTGCCACACTGTCTGTG
TCTGCCGGCGAGAGAGTGACCATTACCTGCAAGGCCAGCCAGAGCGT
GTCCAACGACGTGACCTGGTATCAGCAGAAGCCAGGACAGGCCCCTC
GGCTGCTGATCTACAGCGCCAGCAATAGATACAGCGGCGTGCCCGCC
AGATTTTCCGGCTCTGGATACGGCACCGAGTTCACCTTCACCATCAG
CTCCGTGCAGAGCGAGGACTTCGCTGTGTACTTCTGTCAGCAAGACT
ACAGCTCCTTCGGCCAGGGCACCAAGCTGGAAATCAAGAGAGGAGGC
GGAGGATCTGGTGGCGGAGGAAGTGGCGGAGGCGGTTCTGGCGGTGG
TGGATCTGAGCAGAAGCTGATCTCCGAAGAGGACCTCCAGGTGCAGC
TGGTGGAATCTGGACCTGGTGTTGTGCAGCCTGGCAGAAGCCTGAGA
ATCAGCTGTGCCGTGTCCGGCTTCAGCGTGACCAATTATGGCGTGCA
CTGGGTCCGACAGCCTCCAGGCAAAGGACTGGAATGGCTGGGAGTGA
TTTGGGCTGGCGGCATCACCAACTACAACAGCGCCTTCATGAGCCGG
CTGACCATCAGCAAGGACAACAGCAAGAACACCGTGTACCTGCAGAT
GAACAGCCTGAGAGCCGAGGACACCGCCATGTACTACTGTGCTTCTA
GAGGCGGCCACTACGGCTACGCCCTGGATTATTGGGGACAGGGCACA
CTGGTCACAGTGTCTAGCCCTTGCGTGGGCAGCAACCCCTGCTACAA
TCAGGGCACATGCGAGCCCACCAGCGAGAACCCCTTCTACAGATGTC
TGTGCCCCGCCAAGTTCAACGGCCTGCTGTGTCACATCCTGGACTAC
AGCTTTACCGGCGGAGCCGGCAGAGATATCCCTCCACCTCAGATTGA
GGAAGCCTGCGAGCTGCCTGAGTGTCAGGTTGACGCCGGCAACAAAG
TGTGCAACCTGCAGTGCAACAACCACGCCTGTGGATGGGATGGCGGC
GACTGTAGCCTGAACTTCAACGACCCCTGGAAGAACTGCACCCAGAG
CCTGCAGTGTTGGAAGTACTTTAGCGACGGCCACTGCGACAGCCAGT
GTAATTCTGCCGGATGCCTGTTCGACGGCTTCGACTGCCAACTGACA
GAGGGCCAGTGCAACCCTCTGTACGACCAGTACTGCAAGGACCACTT
CTCCGATGGCCACTGTGACCAGGGCTGTAATAGCGCCGAGTGCGAGT
GGGATGGACTGGATTGTGCCGAGCACGTGCCAGAAAGACTGGCCGCT
GGAACACTGGTGCTGGTGGTTCTTCTGCCTCCTGACCAGCTGCGGAA
CAACAGCTTCCACTTCCTGCGGGAACTGAGCCACGTGCTGCACACCA
ACGTGGTGTTCAAGAGAGATGCCCAGGGACAGCAGATGATCTTCCCC
TACTACGGCCACGAAGAGGAACTGCGGAAGCACCCCATCAAGAGATC
TACAGTCGGCTGGGCCACCTCCAGTCTGCTGCCTGGAACAAGTGGCG
GCAGACAGAGAAGAGAACTGGACCCCATGGACATCCGGGGCAGCATC
GTGTACCTGGAAATCGACAACCGGCAGTGCGTGCAGAGCAGCTCCCA
GTGTTTTCAGAGCGCTACTGACGTGGCCGCCTTTCTGGGAGCACTTG
CTTCTCTGGGCAGCCTGAACATCCCCTACAAGATCGAGGCCGTGAAG
TCCGAGCCTGTGGAACCTCCTCTGCCTTCTCAGCTGCACCTTATGTA
CGTGGCAGCCGCCGCTTTCGTGCTGCTGTTCTTTGTTGGATGCGGAG
TGCTGCTGAGCCGGAAGCGGAGAAGAATGAAGCTGCTGTCCAGCATC
GAGCAGGCCTGTGACATCTGCAGACTGAAGAAACTGAAGTGCAGCAA
AGAAAAGCCCAAGTGCGCCAAGTGCCTGAAGAACAATTGGGAGTGCC
GGTACAGCCCCAAGACCAAGAGATCCCCTCTGACAAGAGCCCACCTG
ACCGAGGTGGAAAGCCGGCTGGAAAGACTCGAGCAGCTGTTCCTGCT
GATCTTTCCACGCGAGGACCTGGACATGATTCTGAAGATGGACTCTC
TGCAGGACATCAAGGCCCTGCTGACCGGCCTGTTCGTGCAGGACAAC
GTGAACAAGGACGCCGTGACCGATAGACTGGCCTCCGTGGAAACCGA
CATGCCCCTGACACTGAGACAGCACAGAATCAGCGCCACCAGCAGCA
GCGAGGAAAGCAGCAACAAGGGCCAGAGACAGCTGACAGTGTCTGCT
GCAGCTGGCGGATCAGGTGGTAGTGGCGGATCTGATGCCCTGGACGA
CTTTGACCTGGATATGCTGGGCAGCGACGCCCTGGATGATTTTGATC
TGGACATGCTCGGCTCCGACGCTCTCGACGATTTCGACCTCGACATG
TTGGGATCCGACGCACTTGATGACTTCGATCTCGATATGCTCGGGTC
CTGAGATCCTTGACTTGCGGCCGCAACTCCCACCTGCAACATGCGTG
ACTGACTGAGGCCGCGACTCTAGAGTCGACCTGCAGGCATGCAAGCT
TGATATCAAGCTTATCGATAATCAACCTCTGGATTACAAAATTTGTG
AAAGATTGACTGGTATTCTTAACTATGTTGCTCCTTTTACGCTATGT
GGATACGCTGCTTTAATGCCTTTGTATCATGCTATTGCTTCCCGTAT
GGCTTTCATTTTCTCCTCCTTGTATAAATCCTGGTTGCTGTCTCTTT
ATGAGGAGTTGTGGCCCGTTGTCAGGCAACGTGGCGTGGTGTGCACT
GTGTTTGCTGACGCAACCCCCACTGGTTGGGGCATTGCCACCACCTG
TCAGCTCCTTTCCGGGACTTTCGCTTTCCCCCTCCCTATTGCCACGG
CGGAACTCATCGCCGCCTGCCTTGCCCGCTGCTGGACAGGGGCTCGG
CTGTTGGGCACTGACAATTCCGTGGTGTTGTCGGGGAAATCATCGTC
CTTTCCTTGGCTGCTCGCCTGTGTTGCCACCTGGATTCTGCGCGGGA
CGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCT
TCGCCCTCAGACGAGTCGGATCTCCCTTTGGGCCGCCTCCCCGCAT.

29. A vector comprising the construct of any one of claims 1-28.

30. The vector of claim 29, wherein the vector is a gammaretroviral vector or lentiviral vector.

31. A protein comprising
a. a scFv;
b. a notch transmembrane domain; and
c. a transcription factor.

32. The protein of claim 31, wherein the scFv is a neuroblastoma-specific ScFv.

33. The protein of any one of claims 31-32, wherein the scFv is a GD2 specific ScFv.

34. The protein of any one of claims 31-33, wherein the scFv is a CD19 specific ScFv.

35. The protein of any one of claims 31-34, wherein the scFv comprises a heavy chain fragment and a light chain fragment.

36. The protein of claim 35, wherein the heavy chain fragment comprises an amino acid sequence of
QVKLQQSGPELVEPGASVKISCKTSGYKFTEYTMHWVKQSHGKSLEW
IGGINPNNGGTNYKQKFKGKATLTVDKSSSTAYMELRSLTSEDSAVY
YCARDTTVPFAYWVQGTTVTVSS;
and
wherein the light chain fragment comprises an amino acid sequence of
DIELTQSPAIMSASPGEKVTMTCSGSSSISYMHWYQQKPVTSPKRWI
YDTSKLASGVPARFSGSGSGTSYSLTISSMEAVDAATYYCHQRSSYP
LTFGAGTQLEIKR.

37. The protein of claim 35, wherein the heavy chain fragment comprises an amino acid sequence of
EVQLVQSGAEVEKPGASVKISCKASGSSFTGYNMNWVRQNIGKSLEWIGA
IDPYYGGTSYNQKFKGRATLTVDKSTSTAYMHLKSLRSEDTAVYYCVSGM
EYWGQGTSVTVSS;
and
wherein the light chain fragment comprises an amino acid sequence of
DVVMTQTPLSLPVTPGEPASISCRSSQSLVHRNGNTYLHWYLQKPGQSPK
LLIHKVSNRFSGVPDRFSGSGSGTDFTLKISRVEAEDLGVYFCSQSTHVP
PLTFGAGTKLELKR.

38. The protein of claim 35, wherein the heavy chain fragment comprises an amino acid sequence of
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSS;
and
wherein the light chain fragment comprises an amino acid sequence of
EIVMTQTPATLSVSAGERVTITCKASQSVSNDVTWYQQKPGQAPRLLIYS
ASNRYSGVPARFSGSGYGTEFTFTISSVQSEDFAVYFCQQDYSSFGQGTK
LEIKR.

39. The protein of claim 35, wherein the heavy chain fragment comprises an amino acid sequence of
QVQLQESGPGLVKPSQTLSITCTVSGFSLASYNIHWVRQPPGKGLEWLGV
IWAGGSTNYNSALMSRLTISKDNSKNQVFLKMSSLTAADTAVYYCAKRSD
DYSWFAYWGQGTLVTVSS;
and
wherein the light chain fragment comprises an amino acid sequence of
ENQMTQSPSSLSASVGDRVTMTCRASSSVSSSYLHWYQQKSGKAPKVWIY
STSNLASGVPSRESGSGSGTDYTLTISSLQPEDFATYYCQQYSGYPITFG
QGTKVEIKR.

40. The protein of claim 35, wherein the heavy chain fragment comprises an amino acid sequence of
LISEEDLEVQLVETGGGVVKPGGSLRLSCAASGFTFSDYYMSWIRQAPGK
GLEWVSYISSSGSTIYYADSVKGRFTISRDNSKNTLYLQMNSLRAEDTAV
YYCARESGYDYVFDYWGQGTLVAVSS;
and
wherein the light chain fragment comprises an amino acid sequence of
DIQMTQSPSTLSAFVGDRVTITCRASQSISSWLAWYQQKPGKAPKLLIYA
ASTLQSGVPSRFSGSGSGTEFTLTISSLQPEDFATYYCQQLNSYPITFGQ
GTRLEIKRILDYSF.

41. The protein of claim 35, wherein the heavy chain fragment comprises an amino acid sequence of
LISEEDLEVQLVESGGGLVQPGGSLRLSCAASGFTFSSFGMHWVRQAPGK
GLEWVAYISSDSSAIYYADTVKGRFTISRDNAKNSLYLQMNSLRDEDTAV
YYCGRGRENIYYGSRLDYWGQGTTVTVSS;
and
wherein the light chain fragment comprises an amino acid sequence of
DIQLTQSPSFLSASVGDRVTITCKASQNVDTNVAWYQQKPGKAPKALIYS
ASYRYSGVPSRFSGSGSGTDFTLTISSLQPEDFATYYCQQYNNYPFTFGQ
GTKLEIKILDYSF.

42. The protein of claim 35, wherein the heavy chain fragment comprises an amino acid sequence of
LISEEDLQVQLQQPGAELVKPGASVKLSCKASGYTFTGYWMHWVKQRPGH
GLEWIGEINPSNGRTNYNERFKSKATLTVDKSSTTAFMQLSGLTSEDSAV
YFCARDYYGTSYNFDYWGQGTTLTVSS;
and
wherein the light chain fragment comprises an amino acid sequence of
DIQMTQSSSSFSVSLGDRVTITCKANEDINNRLAWYQQTPGNSPRLLISG
ATNLVTGVPSRFSGSGSGKDYTLTITSLQAEDFATYYCQQYWSTPFTFGS
GTELEIKVEILDYSF.

43. The protein of any one of claims 31-42, wherein the heavy chain fragment and light chain fragment are joined via a linker.

44. The protein of claim 43, wherein the linker comprises an amino acid sequence of GGGGSGGGGSGGGGS or GGGGSGGGGSGGGGSGGGGS.

45. The protein of any one of claims 31-44, wherein the notch transmembrane domain comprises the amino acid sequence of
ILDYSFTGGAGRDIPPPQIEEACELPECQVDAGNKVCNLQCNNHACGWDG
GDCSLNFNDPWKNCTQSLQCWKYFSDGHCDSQCNSAGCLFDGFDCQLTEG
QCNPLYDQYCKDHFSDGHCDQGCNSAECEWDGLDCAEHVPERLAAGTLVL
VVLLPPDQLRNNSFHFLRELSHVLHTNVVFKRDAQGQQMIFPYYGHEEEL
RKHPIKRSTVGWATSSLLPGTSGGRQRRELDPMDIRGSIVYLEIDNRQCV
QSSSQCFQSATDVAAFLGALASLGSLNIPYKIEAVKSEPVEPPLPSQLHL
MYVAAAAFVLLFFVGCGVLLSRKRRR.

46. The protein of any one of claims 31-45, wherein the notch transmembrane domain further comprises one or more ERRs from the notch extracellular domain.

47. The protein of claim 46, wherein the ERR sequence comprises the amino acid sequence of PCVGSNPCYNQGTCEPTSENPFYRCLCPAKFNGLLCH.

48. The protein of any one of claims 31-47, wherein the transcription factor is a transcription activator.

49. The protein of claim 48, wherein the transcription activator comprises Gal4.

50. The protein of claim 49, wherein the transcription activator comprises a Gal4-VP16 fusion protein.

51. The protein of claim 50, wherein the Gal4-VP64 fusion protein comprises the amino acid sequence of
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVSAAA
GGSGGSGGSDALDDFDLDMLGSDALDDFDLDMLGSDALDDFDLDMLGSDA
LDDFDLDMLGS.

52. The protein of any one of claims 31-51, further comprising a detection agent.

53. The protein of claim 52, wherein the detection agent allows for visual detection or purification.

54. The protein of claim 53, wherein the detection agent is a myc tag, his tag, fluorescent tag, FLAG tag, or hemagglutinin tag.

55. A recombinant cell comprising the construct of any one of claims 1-28 or the vector of any of claims 29-30 or the protein of any one of claims 31-54.

56. The recombinant cell of claim 55, further comprising a response construct comprising
a. an upstream activation sequence (UAS);
b. a promoter controlled by the UAS; and
c. a gene encoding IFNγ operably linked to the promoter.

57. The recombinant cell of claim 56, wherein the response construct further comprises an IRES sequence and/or a detectable moiety.

58. The recombinant cell of any one of claims 56-57, wherein the UAS of the response construct is a Gal4-UAS.

59. The recombinant cell of claim 58, wherein the Gal4-UAS comprises the nucleic acid sequence of
GGAGCACTGTCCTCCGAACGTCGGAGCACTGTCCTCCGAACGTCGGAGC
ACTGTCCTCCGAACGTCGGAGCACTGTCCTCCGAACGGAGCATGTCCTCC
GAACGTCGGAGCACTGTCCTCCGAACG.

60. The recombinant cell of any one of claims 56-59, wherein the recombinant cell is a T cell.

61. The recombinant cell of claim 60, wherein the T cell is TCR deficient T cell.

62. The recombinant cell of claim 60, wherein the T cell is a primary T cell, J76 T cell, or a TCR-deficient J76 T cell.

63. A method of increasing human leukocyte antigen class I (HLA-I) on the surface of a tumor cell in a subject comprising administering to the subject a recombinant cell of any of claims 55-62 or a composition comprising a recombinant cell of any one of claims 55-62.

64. A method of increasing HLA-I on the surface of a tumor cell in a subject comprising administering to the subject one or more of the nucleic acid constructs of claims 1-28, vectors of claims 29-30, or proteins of claims 31-54.

65. The method of any one of claims 63-64, wherein the tumor cell expresses one or more of GD2, B7H3, CD171, and GPC2.

66. The method of any one of claims 63-65, wherein the tumor cell is a neuroblastoma cell, retinoblastoma cell, pediatric sarcoma cell, brain tumor cell, small-cell lung cancer cell, melanoma cell, or soft-tissue sarcoma cell.

67. The method of any one of claims 63-66, wherein the transcription factor binds to the UAS in the response construct.

68. The method of any one of claims 63-66, wherein activation of the UAS activates the promoter operably linked to the IFNγ of the response construct.

69. The method of any of claims 63-68, wherein the recombinant cell produces IFNγ only in the presence of antigen-positive tumor cells.

70. The method of any of claims 63-69, wherein the recombinant cell secretes IFNγ only in the presence of antigen-positive tumor cells.

71. The method of any one of claims 63-70, wherein the recombinant cell is administered to the subject via intravenous, intratumoral, intraperitoneal, or intrathecal routes.

72. The method of any one of claims 63-71, wherein PD-1 and/or PD-L1 expression on the tumor cells is not altered.

73. The method of any one of claims 63-72, wherein PD-1 and/or PD-L1 expression on the tumor cells is not upregulated.


SEQUENCE LISTING (57 sequences)

SEQ ID NO: 1 - protein, 117 aa, synthetic construct
  REGION 1..117: note = synthetic construct; heavy chain fragment
QVKLQQSGPELVEPGASVKISCKTSGYKFTEYTMHWVKQSHGKSLEWIGGINPNNGGTNYKQKFKGKATLTVDKSSSTAYMELRSLTSEDSAVYYCARDTTVPFAYWVQGTTVTVSS

SEQ ID NO: 2 - protein, 107 aa, synthetic construct
  REGION 1..107: note = synthetic construct; light chain fragment
DIELTQSPAIMSASPGEKVTMTCSGSSSISYMHWYQQKPVTSPKRWIYDTSKLASGVPARFSGSGSGTSYSLTISSMEAVDAATYYCHQRSSYPLTFGAGTQLEIKR

SEQ ID NO: 3 - protein, 113 aa, synthetic construct
  REGION 1..113: note = synthetic construct; heavy chain fragment
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSS

SEQ ID NO: 4 - protein, 114 aa, synthetic construct
  REGION 1..114: note = synthetic construct; light chain fragment
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKR

SEQ ID NO: 5 - protein, 119 aa, synthetic construct
  REGION 1..119: note = synthetic construct; heavy chain fragment
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSS

SEQ ID NO: 6 - protein, 105 aa, synthetic construct
  REGION 1..105: note = synthetic construct; light chain fragment
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKR

SEQ ID NO: 7 - protein, 118 aa, synthetic construct
  REGION 1..118: note = synthetic construct; heavy chain fragment
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSS

SEQ ID NO: 8 - protein, 109 aa, synthetic construct
  REGION 1..109: note = synthetic construct; light chain fragment
ENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKR

SEQ ID NO: 9 - protein, 126 aa, synthetic construct
  REGION 1..126: note = synthetic construct; heavy chain fragment
LISEEDLEVQLVETGGGVVKPGGSLRLSCAASGFTFSDYYMSWIRQAPGKGLEWVSYISSSGSTIYYADSVKGRFTISRDNSKNTLYLQMNSLRAEDTAVYYCARESGYDYVFDYWGQGTLVAVSS

SEQ ID NO: 10 - protein, 114 aa, synthetic construct
  REGION 1..114: note = synthetic construct; light chain fragment
DIQMTQSPSTLSAFVGDRVTITCRASQSISSWLAWYQQKPGKAPKLLIYAASTLQSGVPSRFSGSGSGTEFTLTISSLQPEDFATYYCQQLNSYPITFGQGTRLEIKRILDYSF

SEQ ID NO: 11 - protein, 129 aa, synthetic construct
  REGION 1..129: note = synthetic construct; heavy chain fragment
LISEEDLEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 12 - protein, 113 aa, synthetic construct
  REGION 1..113: note = synthetic construct; light chain fragment
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKILDYSF

SEQ ID NO: 13 - protein, 127 aa, synthetic construct
  REGION 1..127: note = synthetic construct; heavy chain fragment
LISEEDLQVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSS

SEQ ID NO: 14 - protein, 115 aa, synthetic construct
  REGION 1..115: note = synthetic construct; light chain fragment
DIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIKVEILDYSF

SEQ ID NO: 15 - protein, 15 aa, synthetic construct
  REGION 1..15: note = synthetic construct; linker
GGGGSGGGGSGGGGS

SEQ ID NO: 16 - protein, 20 aa, synthetic construct
  REGION 1..20: note = synthetic construct; linker
GGGGSGGGGSGGGGSGGGGS

SEQ ID NO: 17 - protein, 50 aa, synthetic construct
  REGION 1..50: note = synthetic construct; linker
  REPEAT 1..5: note = up to 9 copies of the GGGGS repeat can be deleted
GGGGSGGGGSGGGGSGGGGSGGGGSGGGGSGGGGSGGGGSGGGGSGGGGS

SEQ ID NO: 18 - protein, 18 aa, synthetic construct
  REGION 1..18: note = synthetic construct; linker
GGSSRSSSSGGGGSGGGG

SEQ ID NO: 19 - protein, 326 aa, synthetic construct
  REGION 1..326: note = synthetic construct; notch transmembrane domain
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 20 - protein, 37 aa, synthetic construct
  REGION 1..37: note = synthetic construct; EGF repeat sequence
PCVGSNPCYNQGTCEPTSENPFYRCLCPAKFNGLLCH

SEQ ID NO: 21 - protein, 211 aa, synthetic construct
  REGION 1..211: note = synthetic construct; Gal4-VP64 fusion protein
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 22 - protein, 236 aa, synthetic construct
  REGION 1..236: note = synthetic construct; mcherry peptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 23 - protein, 233 aa, synthetic construct
  REGION 1..233: note = synthetic construct; BFP
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 24 - protein, 19 aa, synthetic construct
  REGION 1..19: note = synthetic construct; signaling peptide
MDWTWRVFCLLAVAPGAHS

SEQ ID NO: 25 - protein, 20 aa, synthetic construct
  REGION 1..20: note = synthetic construct; signaling peptide
MVLQTQVFISLLLWISGAYG

SEQ ID NO: 26 - protein, 19 aa, synthetic construct
  REGION 1..19: note = synthetic construct; signaling peptide
MAWALLLLSLLTQGTGSWA

SEQ ID NO: 27 - protein, 21 aa, synthetic construct
  REGION 1..21: note = synthetic construct; signaling peptide
MALPVTALLLPLALLLHAARP

SEQ ID NO: 28 - protein, 18 aa, synthetic construct
  REGION 1..18: note = synthetic construct; signaling peptide
MLRLLLALNLFPSIQVTG

SEQ ID NO: 29 - protein, 20 aa, synthetic construct
  REGION 1..20: note = synthetic construct; signaling peptide
MYRMQLLSCIALSLALVTNS

SEQ ID NO: 30 - DNA, 2550 bp, synthetic construct
  misc_feature 1..2550: note = synthetic construct; fusion protein
  source 1..2550: mol_type = other DNA
atggccctgcctgttacagctctgctgctgcctctggctctgcttctgcatgccgctagacctgagatcgtgatgacacagacccctgccacactgtctgtgtctgccggcgagagagtgaccattacctgcaaggccagccagagcgtgtccaacgacgtgacctggtatcagcagaagccaggacaggcccctcggctgctgatctacagcgccagcaatagatacagcggcgtgcccgccagattttccggctctggatacggcaccgagttcaccttcaccatcagctccgtgcagagcgaggacttcgctgtgtacttctgtcagcaagactacagctccttcggccagggcaccaagctggaaatcaagagaggaggcggaggatctggtggcggaggaagtggcggaggcggttctggcggtggtggatctgagcagaagctgatctccgaagaggacctccaggtgcagctggtggaatctggacctggtgttgtgcagcctggcagaagcctgagaatcagctgtgccgtgtccggcttcagcgtgaccaattatggcgtgcactgggtccgacagcctccaggcaaaggactggaatggctgggagtgatttgggctggcggcatcaccaactacaacagcgccttcatgagccggctgaccatcagcaaggacaacagcaagaacaccgtgtacctgcagatgaacagcctgagagccgaggacaccgccatgtactactgtgcttctagaggcggccactacggctacgccctggattattggggacagggcacactggtcacagtgtctagcccttgcgtgggcagcaacccctgctacaatcagggcacatgcgagcccaccagcgagaaccccttctacagatgtctgtgccccgccaagttcaacggcctgctgtgtcacatcctggactacagctttaccggcggagccggcagagatatccctccacctcagattgaggaagcctgcgagctgcctgagtgtcaggttgacgccggcaacaaagtgtgcaacctgcagtgcaacaaccacgcctgtggatgggatggcggcgactgtagcctgaacttcaacgacccctggaagaactgcacccagagcctgcagtgttggaagtactttagcgacggccactgcgacagccagtgtaattctgccggatgcctgttcgacggcttcgactgccaactgacagagggccagtgcaaccctctgtacgaccagtactgcaaggaccacttctccgatggccactgtgaccagggctgtaatagcgccgagtgcgagtgggatggactggattgtgccgagcacgtgccagaaagactggccgctggaacactggtgctggtggttcttctgcctcctgaccagctgcggaacaacagcttccacttcctgcgggaactgagccacgtgctgcacaccaacgtggtgttcaagagagatgcccagggacagcagatgatcttcccctactacggccacgaagaggaactgcggaagcaccccatcaagagatctacagtcggctgggccacctccagtctgctgcctggaacaagtggcggcagacagagaagagaactggaccccatggacatccggggcagcatcgtgtacctggaaatcgacaaccggcagtgcgtgcagagcagctcccagtgttttcagagcgctactgacgtggccgcctttctgggagcacttgcttctctgggcagcctgaacatcccctacaagatcgaggccgtgaagtccgagcctgtggaacctcctctgccttctcagctgcaccttatgtacgtggcagccgccgctttcgtgctgctgttctttgttggatgcggagtgctgctgagccggaagcggagaagaatgaagctgctgtccagcatcgagcaggcctgtgacatctgcagactgaagaaactgaagtgcagcaaagaaaagcccaagtgcgccaagtgcctgaagaacaattgggagtgccggtacagccccaagaccaagagatcccctctgacaagagcccacctgaccgaggtggaaagccggctggaaagactcgagcagctgttcctgctgatctttccacgcgaggacctggacatgattctgaagatggactctctgcaggacatcaaggccctgctgaccggcctgttcgtgcaggacaacgtgaacaaggacgccgtgaccgatagactggcctccgtggaaaccgacatgcccctgacactgagacagcacagaatcagcgccaccagcagcagcgaggaaagcagcaacaagggccagagacagctgacagtgtctgctgcagctggcggatcaggtggtagtggcggatctgatgccctggacgactttgacctggatatgctgggcagcgacgccctggatgattttgatctggacatgctcggctccgacgctctcgacgatttcgacctcgacatgttgggatccgacgcacttgatgacttcgatctcgatatgctcgggtcctga

SEQ ID NO: 31 - DNA, 2544 bp, synthetic construct
  misc_feature 1..2544: note = synthetic construct; fusion protein
  source 1..2544: mol_type = other DNA
atggccctgcctgttacagctctgctgctgcctctggctctgcttctgcatgccgctagacctgagcagaagctgatctccgaagaggacctccaggtccagctgcaagaatctggccctggcctggtcaagcctagccagacactgagcatcacctgtaccgtgtccggctttagcctggccagctacaacatccactgggtccgacagcctccaggcaaaggactggaatggctgggagtgatttgggctggcggcagcaccaactacaacagcgccctgatgagccggctgaccatcagcaaggacaacagcaagaaccaggtgttcctgaagatgagcagcctgacagccgccgataccgccgtgtactactgtgccaagagaagcgacgactacagttggttcgcctactggggccagggcacactggttacagtttctagcggaggcggaggatctggtggcggaggaagtggcggaggcggttctgagaatcagatgacacagagccccagcagcctgtctgcctctgtgggagatagagtgaccatgacctgcagagcctccagctccgtgtctagcagctacctgcactggtatcagcagaagtccggcaaggcccctaaagtgtggatctacagcaccagcaatctggccagcggcgtgccaagcagattttctggaagcggcagcggcaccgactacaccctgaccatatctagcctgcagcctgaggacttcgccacctactactgccagcagtacagcggctaccccatcacatttggacagggcaccaaggtggaaatcaagaggccttgcgtgggcagcaacccctgctacaatcagggcacatgcgagcccaccagcgagaaccccttctacagatgtctgtgccccgccaagttcaacggcctgctgtgtcacatcctggactacagctttaccggcggagccggcagagatatccctccacctcagattgaggaagcctgcgagctgcctgagtgtcaggttgacgccggcaacaaagtgtgcaacctgcagtgcaacaaccacgcctgtggatgggatggcggcgactgtagcctgaacttcaacgacccctggaagaactgcacccagagcctgcagtgttggaagtactttagcgacggccactgcgacagccagtgtaattctgccggatgcctgttcgacggcttcgactgccaactgacagagggccagtgcaaccctctgtacgaccagtactgcaaggaccacttctccgatggccactgtgaccagggctgtaatagcgccgagtgcgagtgggatggactggattgtgccgagcacgtgccagaaagactggccgctggaacactggtgctggtggttcttctgcctcctgaccagctgcggaacaacagcttccacttcctgcgggaactgagccacgtgctgcacaccaacgtggtgttcaagagagatgcccagggacagcagatgatcttcccctactacggccacgaagaggaactgcggaagcaccccatcaagagatctacagtcggctgggccacctccagtctgctgcctggaacaagtggcggcagacagagaagagaactggaccccatggacatccggggcagcatcgtgtacctggaaatcgacaaccggcagtgcgtgcagagcagctcccagtgttttcagagcgctactgacgtggccgcctttctgggagcacttgcttctctgggcagcctgaacatcccctacaagatcgaggccgtgaagtccgagcctgtggaacctcctctgccttctcagctgcaccttatgtacgtggcagccgccgctttcgtgctgctgttctttgttggatgcggagtgctgctgagccggaagcggagaagaatgaagctgctgtccagcatcgagcaggcctgtgacatctgcagactgaagaaactgaagtgcagcaaagaaaagcccaagtgcgccaagtgcctgaagaacaattgggagtgccggtacagccccaagaccaagagatcccctctgacaagagcccacctgaccgaggtggaaagccggctggaaagactcgagcagctgttcctgctgatctttccacgcgaggacctggacatgattctgaagatggactctctgcaggacatcaaggccctgctgaccggcctgttcgtgcaggacaacgtgaacaaggacgccgtgaccgatagactggcctccgtggaaaccgacatgcccctgacactgagacagcacagaatcagcgccaccagcagcagcgaggaaagcagcaacaagggccagagacagctgacagtgtctgctgcagctggcggatcaggtggtagtggcggatctgatgccctggacgactttgacctggatatgctgggcagcgacgccctggatgattttgatctggacatgctcggctccgacgctctcgacgatttcgacctcgacatgttgggatccgacgcacttgatgacttcgatctcgatatgctcgggtcctga

SEQ ID NO: 32 - DNA, 11514 bp, synthetic construct
  misc_feature 1..11514: note = synthetic construct; fusion protein
  source 1..11514: mol_type = other DNA
cgataccgtcgaccaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctagctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggtgatcttcagacctggacgatatatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaattctcacgcgtcaagtggagcaaggcaggtggacagtggatcatggccctgcctgttacagctctgctgctgcctctggctctgcttctgcatgccgctagacctgagatcgtgatgacacagacccctgccacactgtctgtgtctgccggcgagagagtgaccattacctgcaaggccagccagagcgtgtccaacgacgtgacctggtatcagcagaagccaggacaggcccctcggctgctgatctacagcgccagcaatagatacagcggcgtgcccgccagattttccggctctggatacggcaccgagttcaccttcaccatcagctccgtgcagagcgaggacttcgctgtgtacttctgtcagcaagactacagctccttcggccagggcaccaagctggaaatcaagagaggaggcggaggatctggtggcggaggaagtggcggaggcggttctggcggtggtggatctgagcagaagctgatctccgaagaggacctccaggtgcagctggtggaatctggacctggtgttgtgcagcctggcagaagcctgagaatcagctgtgccgtgtccggcttcagcgtgaccaattatggcgtgcactgggtccgacagcctccaggcaaaggactggaatggctgggagtgatttgggctggcggcatcaccaactacaacagcgccttcatgagccggctgaccatcagcaaggacaacagcaagaacaccgtgtacctgcagatgaacagcctgagagccgaggacaccgccatgtactactgtgcttctagaggcggccactacggctacgccctggattattggggacagggcacactggtcacagtgtctagcccttgcgtgggcagcaacccctgctacaatcagggcacatgcgagcccaccagcgagaaccccttctacagatgtctgtgccccgccaagttcaacggcctgctgtgtcacatcctggactacagctttaccggcggagccggcagagatatccctccacctcagattgaggaagcctgcgagctgcctgagtgtcaggttgacgccggcaacaaagtgtgcaacctgcagtgcaacaaccacgcctgtggatgggatggcggcgactgtagcctgaacttcaacgacccctggaagaactgcacccagagcctgcagtgttggaagtactttagcgacggccactgcgacagccagtgtaattctgccggatgcctgttcgacggcttcgactgccaactgacagagggccagtgcaaccctctgtacgaccagtactgcaaggaccacttctccgatggccactgtgaccagggctgtaatagcgccgagtgcgagtgggatggactggattgtgccgagcacgtgccagaaagactggccgctggaacactggtgctggtggttcttctgcctcctgaccagctgcggaacaacagcttccacttcctgcgggaactgagccacgtgctgcacaccaacgtggtgttcaagagagatgcccagggacagcagatgatcttcccctactacggccacgaagaggaactgcggaagcaccccatcaagagatctacagtcggctgggccacctccagtctgctgcctggaacaagtggcggcagacagagaagagaactggaccccatggacatccggggcagcatcgtgtacctggaaatcgacaaccggcagtgcgtgcagagcagctcccagtgttttcagagcgctactgacgtggccgcctttctgggagcacttgcttctctgggcagcctgaacatcccctacaagatcgaggccgtgaagtccgagcctgtggaacctcctctgccttctcagctgcaccttatgtacgtggcagccgccgctttcgtgctgctgttctttgttggatgcggagtgctgctgagccggaagcggagaagaatgaagctgctgtccagcatcgagcaggcctgtgacatctgcagactgaagaaactgaagtgcagcaaagaaaagcccaagtgcgccaagtgcctgaagaacaattgggagtgccggtacagccccaagaccaagagatcccctctgacaagagcccacctgaccgaggtggaaagccggctggaaagactcgagcagctgttcctgctgatctttccacgcgaggacctggacatgattctgaagatggactctctgcaggacatcaaggccctgctgaccggcctgttcgtgcaggacaacgtgaacaaggacgccgtgaccgatagactggcctccgtggaaaccgacatgcccctgacactgagacagcacagaatcagcgccaccagcagcagcgaggaaagcagcaacaagggccagagacagctgacagtgtctgctgcagctggcggatcaggtggtagtggcggatctgatgccctggacgactttgacctggatatgctgggcagcgacgccctggatgattttgatctggacatgctcggctccgacgctctcgacgatttcgacctcgacatgttgggatccgacgcacttgatgacttcgatctcgatatgctcgggtcctgagatccttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 33 - DNA, 11508 bp, synthetic construct
  misc_feature 1..11508: note = synthetic construct; fusion protein
  source 1..11508: mol_type = other DNA
cgataccgtcgaccaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctagctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggtgatcttcagacctggacgatatatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaattctcacgcgtcaagtggagcaaggcaggtggacagtggatcatggccctgcctgttacagctctgctgctgcctctggctctgcttctgcatgccgctagacctgagcagaagctgatctccgaagaggacctccaggtccagctgcaagaatctggccctggcctggtcaagcctagccagacactgagcatcacctgtaccgtgtccggctttagcctggccagctacaacatccactgggtccgacagcctccaggcaaaggactggaatggctgggagtgatttgggctggcggcagcaccaactacaacagcgccctgatgagccggctgaccatcagcaaggacaacagcaagaaccaggtgttcctgaagatgagcagcctgacagccgccgataccgccgtgtactactgtgccaagagaagcgacgactacagttggttcgcctactggggccagggcacactggttacagtttctagcggaggcggaggatctggtggcggaggaagtggcggaggcggttctgagaatcagatgacacagagccccagcagcctgtctgcctctgtgggagatagagtgaccatgacctgcagagcctccagctccgtgtctagcagctacctgcactggtatcagcagaagtccggcaaggcccctaaagtgtggatctacagcaccagcaatctggccagcggcgtgccaagcagattttctggaagcggcagcggcaccgactacaccctgaccatatctagcctgcagcctgaggacttcgccacctactactgccagcagtacagcggctaccccatcacatttggacagggcaccaaggtggaaatcaagaggccttgcgtgggcagcaacccctgctacaatcagggcacatgcgagcccaccagcgagaaccccttctacagatgtctgtgccccgccaagttcaacggcctgctgtgtcacatcctggactacagctttaccggcggagccggcagagatatccctccacctcagattgaggaagcctgcgagctgcctgagtgtcaggttgacgccggcaacaaagtgtgcaacctgcagtgcaacaaccacgcctgtggatgggatggcggcgactgtagcctgaacttcaacgacccctggaagaactgcacccagagcctgcagtgttggaagtactttagcgacggccactgcgacagccagtgtaattctgccggatgcctgttcgacggcttcgactgccaactgacagagggccagtgcaaccctctgtacgaccagtactgcaaggaccacttctccgatggccactgtgaccagggctgtaatagcgccgagtgcgagtgggatggactggattgtgccgagcacgtgccagaaagactggccgctggaacactggtgctggtggttcttctgcctcctgaccagctgcggaacaacagcttccacttcctgcgggaactgagccacgtgctgcacaccaacgtggtgttcaagagagatgcccagggacagcagatgatcttcccctactacggccacgaagaggaactgcggaagcaccccatcaagagatctacagtcggctgggccacctccagtctgctgcctggaacaagtggcggcagacagagaagagaactggaccccatggacatccggggcagcatcgtgtacctggaaatcgacaaccggcagtgcgtgcagagcagctcccagtgttttcagagcgctactgacgtggccgcctttctgggagcacttgcttctctgggcagcctgaacatcccctacaagatcgaggccgtgaagtccgagcctgtggaacctcctctgccttctcagctgcaccttatgtacgtggcagccgccgctttcgtgctgctgttctttgttggatgcggagtgctgctgagccggaagcggagaagaatgaagctgctgtccagcatcgagcaggcctgtgacatctgcagactgaagaaactgaagtgcagcaaagaaaagcccaagtgcgccaagtgcctgaagaacaattgggagtgccggtacagccccaagaccaagagatcccctctgacaagagcccacctgaccgaggtggaaagccggctggaaagactcgagcagctgttcctgctgatctttccacgcgaggacctggacatgattctgaagatggactctctgcaggacatcaaggccctgctgaccggcctgttcgtgcaggacaacgtgaacaaggacgccgtgaccgatagactggcctccgtggaaaccgacatgcccctgacactgagacagcacagaatcagcgccaccagcagcagcgaggaaagcagcaacaagggccagagacagctgacagtgtctgctgcagctggcggatcaggtggtagtggcggatctgatgccctggacgactttgacctggatatgctgggcagcgacgccctggatgattttgatctggacatgctcggctccgacgctctcgacgatttcgacctcgacatgttgggatccgacgcacttgatgacttcgatctcgatatgctcgggtcctgagatccttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 34 - protein, 28 aa, synthetic construct
  REGION 1..28: note = synthetic construct; Gal4-UAS
GALSSERRSTVLRTSEHCPPNVGALSSE

SEQ ID NO: 35 - DNA, 498 bp, synthetic construct
  misc_feature 1..498: note = synthetic construct; IFNgamma
  source 1..498: mol_type = other DNA
atgaaatacacttcctatatactcgcttttcaactgtgcatcgtgcttggtagcttgggctgctattgtcaggacccctatgtgaaagaagctgagaaccttaagaagtattttaatgctggtcactctgacgtggcggacaatgggacattgttcctgggtattttgaagaattggaaggaagaatcagatagaaaaataatgcagtcacagatcgtgtccttctacttcaaacttttcaaaaatttcaaggacgaccagtccattcagaagtcagttgaaacaatcaaggaagacatgaacgtgaaatttttcaatagcaataaaaagaaaagggatgattttgagaagttgacaaattactccgtgactgacctcaacgtccaaagaaaagctatacacgagttgatccaagttatggccgagttgagtccggcggcgaaaacaggaaaacgaaagagatcccaaatgctgtttagaggccgccgcgcaagtcag

SEQ ID NO: 36 - protein, 166 aa, synthetic construct
  REGION 1..166: note = synthetic construct; IFNgamma
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 37 - DNA, 11753 bp, synthetic construct
  misc_feature 1..11753: note = synthetic construct; fusion protein
  source 1..11753: mol_type = other DNA
cgataccgtcgaccaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctagctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggtgatcttcagacctggacgatatatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgggaccgatccagcctctcgacattcgttggatccgccaccatgaaatacacttcctatatactcgcttttcaactgtgcatcgtgcttggtagcttgggctgctattgtcaggacccctatgtgaaagaagctgagaaccttaagaagtattttaatgctggtcactctgacgtggcggacaatgggacattgttcctgggtattttgaagaattggaaggaagaatcagatagaaaaataatgcagtcacagatcgtgtccttctacttcaaacttttcaaaaatttcaaggacgaccagtccattcagaagtcagttgaaacaatcaaggaagacatgaacgtgaaatttttcaatagcaataaaaagaaaagggatgattttgagaagttgacaaattactccgtgactgacctcaacgtccaaagaaaagctatacacgagttgatccaagttatggccgagttgagtccggcggcgaaaacaggaaaacgaaagagatcccaaatgctgtttagaggccgccgcgcaagtcagtagggatccttgacttgcggcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgagcgctggcgggtccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggccgcaactcccacctgcaacatgcgtgactgactgaggccgcgactctagagtcgacctgcacgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaatgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 38 - protein, 239 aa, synthetic construct
  REGION 1..239: note = synthetic construct; heavy and light chain fusion
QVKLQQSGPELVEPGASVKISCKTSGYKFTEYTMHWVKQSHGKSLEWIGGINPNNGGTNYKQKFKGKATLTVDKSSSTAYMELRSLTSEDSAVYYCARDTTVPFAYWVQGTTVTVSSGGGGSGGGGSGGGGSDIELTQSPAIMSASPGEKVTMTCSGSSSISYMHWYQQKPVTSPKRWIYDTSKLASGVPARFSGSGSGTSYSLTISSMEAVDAATYYCHQRSSYPLTFGAGTQLEIKR

SEQ ID NO: 39 - protein, 276 aa, synthetic construct
  REGION 1..276: note = synthetic construct; heavy and light chain fusion
QVKLQQSGPELVEPGASVKISCKTSGYKFTEYTMHWVKQSHGKSLEWIGGINPNNGGTNYKQKFKGKATLTVDKSSSTAYMELRSLTSEDSAVYYCARDTTVPFAYWVQGTTVTVSSGGGGSGGGGSGGGGSDIELTQSPAIMSASPGEKVTMTCSGSSSISYMHWYQQKPVTSPKRWIYDTSKLASGVPARFSGSGSGTSYSLTISSMEAVDAATYYCHQRSSYPLTFGAGTQLEIKRPCVGSNPCYNQGTCEPTSENPFYRCLCPAKFNGLLCH

SEQ ID NO: 40 - protein, 247 aa, synthetic construct
  REGION 1..247: note = synthetic construct; heavy and light chain fusion
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSGGGGSGGGGSGGGGSGGGGSDVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKR

SEQ ID NO: 41 - protein, 284 aa, synthetic construct
  REGION 1..284: note = synthetic construct; heavy and light chain fusion
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSGGGGSGGGGSGGGGSGGGGSDVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRPCVGSNPCYNQGTCEPTSENPFYRCLCPAKFNGLLCH

SEQ ID NO: 42 - protein, 244 aa, synthetic construct
  REGION 1..244: note = synthetic construct; heavy and light chain fusion
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKR

SEQ ID NO: 43 - protein, 281 aa, synthetic construct
  REGION 1..281: note = synthetic construct; heavy and light chain fusion
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSEIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRPCVGSNPCYNQGTCEPTSENPFYRCLCPAKFNGLLCH

SEQ ID NO: 44 - protein, 244 aa, synthetic construct
  REGION 1..244: note = synthetic construct; heavy and light chain fusion
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRGGGGSGGGGSGGGGSGGGGSQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSS

SEQ ID NO: 45 - protein, 281 aa, synthetic construct
  REGION 1..281: note = synthetic construct; heavy and light chain fusion
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRGGGGSGGGGSGGGGSGGGGSQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSPCVGSNPCYNQGTCEPTSENPFYRCLCPAKFNGLLCH

SEQ ID NO: 46 - protein, 242 aa, synthetic construct
  REGION 1..242: note = synthetic construct; heavy and light chain fusion
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKR

SEQ ID NO: 47 - protein, 279 aa, synthetic construct
  REGION 1..279: note = synthetic construct; heavy and light chain fusion
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRPCVGSNPCYNQGTCEPTSENPFYRCLCPAKFNGLLCH

SEQ ID NO: 48 - protein, 255 aa, synthetic construct
  REGION 1..255: note = synthetic construct; heavy and light chain fusion
LISEEDLEVQLVETGGGVVKPGGSLRLSCAASGFTFSDYYMSWIRQAPGKGLEWVSYISSSGSTIYYADSVKGRFTISRDNSKNTLYLQMNSLRAEDTAVYYCARESGYDYVFDYWGQGTLVAVSSGGGGSGGGGSGGGGSDIQMTQSPSTLSAFVGDRVTITCRASQSISSWLAWYQQKPGKAPKLLIYAASTLQSGVPSRFSGSGSGTEFTLTISSLQPEDFATYYCQQLNSYPITFGQGTRLEIKRILDYSF

SEQ ID NO: 49 - protein, 257 aa, synthetic construct
  REGION 1..257: note = synthetic construct; heavy and light chain fusion
LISEEDLEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKILDYSF

SEQ ID NO: 50 - protein, 257 aa, synthetic construct
  REGION 1..257: note = synthetic construct; heavy and light chain fusion
LISEEDLQVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSSGGGGSGGGGSGGGGSDIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIKVEILDYSF

SEQ ID NO: 51 - protein, 281 aa, synthetic construct; identical to SEQ ID NO: 45
  REGION 1..281: note = synthetic construct; heavy and light chain fusion

SEQ ID NO: 52 - protein, 326 aa, synthetic construct; identical to SEQ ID NO: 19
  REGION 1..326: note = synthetic construct; synNotch

SEQ ID NO: 53 - protein, 211 aa, synthetic construct; identical to SEQ ID NO: 21
  REGION 1..211: note = synthetic construct; Gal4-VP64

SEQ ID NO: 54 - protein, 28 aa, synthetic construct
  REGION 1..28: note = synthetic construct; Gal4-UAS
GALSSERRSTVLRTSEHCPPNVGALSSE

SEQ ID NO: 55 - protein, 166 aa, synthetic construct; identical to SEQ ID NO: 36
  REGION 1..166: note = synthetic construct; IFNgamma open reading frame

SEQ ID NO: 56 - protein, 236 aa, synthetic construct; identical to SEQ ID NO: 22
  REGION 1..236: note = synthetic construct; mcherry

SEQ ID NO: 57 - protein, 233 aa, synthetic construct; identical to SEQ ID NO: 23
  REGION 1..233: note = synthetic construct; BFP